(19) United States
(12) Patent Application Publication
(10) Pub. No.: US 2008/0226940 A1
(43) Pub. Date: Sep. 18, 2008

(54) DIBENZO[C,G]TRIPHENYLENE DERIVATIVE AND ORGANIC ELECTROLUMINESCENCE DEVICE USING THE SAME

(75) Inventors: Yoriyuki Takashima, Chiba (JP); Masakazu Funahashi, Chiba (JP)

Correspondence Address: OBLON, SPIVAK, MCCLELLAND MAIER & NEUSTADT, P.C., 1940 DUKE STREET, ALEXANDRIA, VA, 22314, US

(73) Assignee: IDEMITSU KOSAN CO.,LTD., Tokyo (JP)

(21) Appl. No.: 11/925,307

(22) Filed: May 13, 2008

(30) Foreign Application Priority Data
Nov. 29, 2006 (JP) 2006-321435

(51) Int. Cl. B32B 9/00; C07C 13/28; C09K 11/00

(52) U.S. Cl. 428/690; 585/26

(57) ABSTRACT

The present invention relates to a dibenzo[c,g]triphenylene derivative, a material for an organic EL device comprising the same, a light emitting material for an organic EL device, a light emitting organic solution, an organic EL device in which an organic compound layer comprising a single layer or plural layers including at least a light emitting layer is interposed between a pair of electrodes, wherein at least one layer in the organic compound layer described above contains at least one kind of the dibenzo[c,g]triphenylene derivative described above and an equipment comprising the same. The present invention provides a novel compound which is useful as a constitutional component for an organic EL device, and a practical organic EL device which is excellent in a color purity and reduced in an operating voltage and which has a small leaked electric current is materialized by using the above compound.

### BACKGROUND OF THE INVENTION

1. Field of the Invention

The present invention relates to an organic electroluminescence device comprising a dibenzo[c,g]triphenylene derivative, specifically to an organic electroluminescence device which is extended in a lifetime and provided with a high current efficiency by using a dibenzo[c,g]triphenylene derivative for a light emitting layer.

2. Related Art

An organic electroluminescence device (hereinafter “electroluminescence” shall be abbreviated as EL) is a spontaneous light emitting device making use of the principle that a fluorescent substance emits light by recombination energy of holes injected from an anode and electrons injected from a cathode by applying an electric field. Since an organic EL device of a laminate type operated at a low voltage was reported by C. W. Tang et al. of Eastman Kodak Company (C. W. Tang and S. A. Vanslyke, Applied Physics Letters, Vol. 51, p. 913, 1987 and the like), researches on organic EL devices comprising organic materials as structural materials have actively been carried out. Tang et al. use tris(8-quinolinolato)aluminum for the light emitting layer and a triphenyldiamine derivative for the hole transporting layer. The advantages of the laminate structure include an elevation in an efficiency of injecting holes into a light emitting layer, a rise in a production efficiency of excitons produced by blocking electrons injected from a cathode to recombine them and shutting up of excitons produced in a light emitting layer. As shown in the above example, a two-layer type comprising a hole transporting (injecting) layer and an electron transporting and light emitting layer and a three-layer type comprising a hole transporting (injecting) layer, a light emitting layer and an electron transporting (injecting) layer are well known as the device structures of an organic EL device. In the above laminate type structural devices, device structures and forming methods are studied in order to enhance a recombination efficiency of holes and electrons injected.

Known as light emitting materials are light emitting materials such as chelate complexes such as a tris(8-quinolinolato)aluminum complex, coumarin derivatives, tetraphenylbutadiene derivatives, bisstyrylarylene derivatives, oxadiazole derivatives and the like. It is reported that light emission of a blue color to a red color in a visible region is obtained from them, and it is expected that a color display device is materialized (for example, Patent Document 1, Patent Document 2, Patent Document 3 and the like).

Devices prepared by using bisanthracene derivatives as a light emitting material are disclosed in Patent Document 4 and Patent Document 5. Bisanthracene is used as a blue light emitting material, but an efficiency and a lifetime thereof do not reach practicable levels and are unsatisfactory.

Also, devices prepared by using symmetric pyrene derivatives as a light emitting material are disclosed in Patent Document 6, Patent Document 7, Patent Document 8 and Patent Document 9. The above symmetric pyrene derivatives are used as a blue light emitting material, but a lifetime of the devices has been required to be improved.

A [5] helicene derivative is disclosed in a patent document 10 as a material having a hole transporting property or an electron transporting ability in addition to a light emitting material, but the specific physical property values are not described therein, and a color purity and a device lifetime as a light emitting material have been required to be improved.

### Patent Document 1: Japanese Patent Application Laid-Open No. 239655/1996

### Patent Document 2: Japanese Patent Application Laid-Open No. 138561/1995

### Patent Document 3: Japanese Patent Application Laid-Open No. 200889/1991

### Patent Document 4: Japanese Patent No. 3008897

### Patent Document 5: Japanese Patent Application Laid-Open No. 12600/1996

### Patent Document 6: Japanese Patent Application Laid-Open No. 118682/2001

### Patent Document 7: Japanese Patent Application Laid-Open No. 63988/2002

### Patent Document 8: Japanese Patent Application Laid-Open No. 75567/2004

### Patent Document 9: Japanese Patent Application Laid-Open No. 83481/2004

### Patent Document 10: International Publication No. 03/051092

### SUMMARY OF THE INVENTION

The present invention has been made in order to solve the problems described above, and an object of the present invention is to provide a novel compound which is useful as a constitutional component of an organic EL device and materialize a practical organic EL device which is reduced in an operating voltage, provided with an excellent color purity and extended in a lifetime by using the above compound.

Intensive researches repeated by the present inventors in order to achieve the object described above have resulted in finding that light emission having a high current efficiency, a high color purity and a long lifetime is obtained by using a dibenzo[c,g]triphenylene derivative for an organic EL device, and thus they have come to complete the present invention.

That is, the present invention provides a dibenzo[c,g]triphenylene derivative, a material for an organic EL device comprising the above derivative, a light emitting material for an organic EL device and a light emitting organic solution.

Further, the present invention provides an organic EL device in which an organic compound layer comprising a single layer or plural layers including at least a light emitting layer is interposed between a pair of electrodes, wherein at least one layer in the organic compound layer described above contains at least one kind of the dibenzo[c,g]triphenylene derivative described above and an equipment comprising the same.

A practical organic EL device which has a low operating voltage, a long lifetime, a high current efficiency and an excellent color purity is obtained by using the dibenzo[c, g]triphenylene derivative of the present invention as a material for the organic EL device.

### BRIEF DESCRIPTION OF THE DRAWINGS

FIG. 1 is a cross section showing the embodiment of the organic EL device of the present invention.

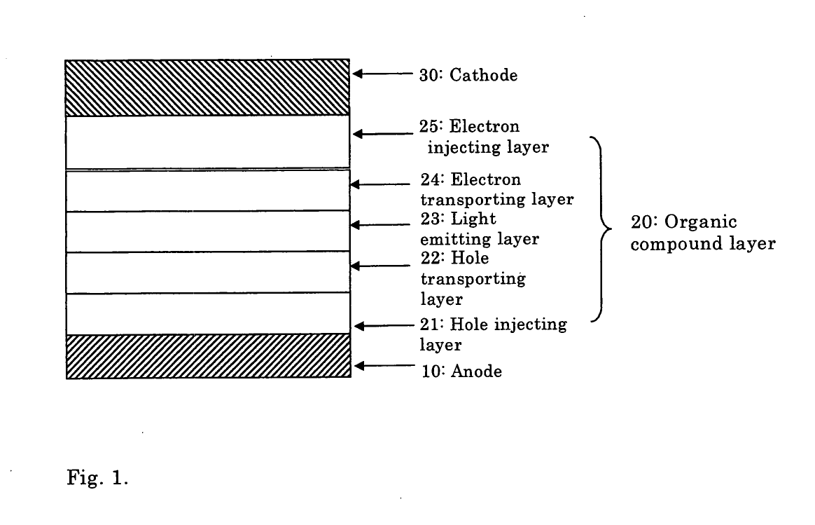

FIG. 2 is a UV light absorption spectrum (◯) and a florescence emission spectrum () of the dibenzo[c,g]triphenylene derivative obtained in Synthetic Example 1.

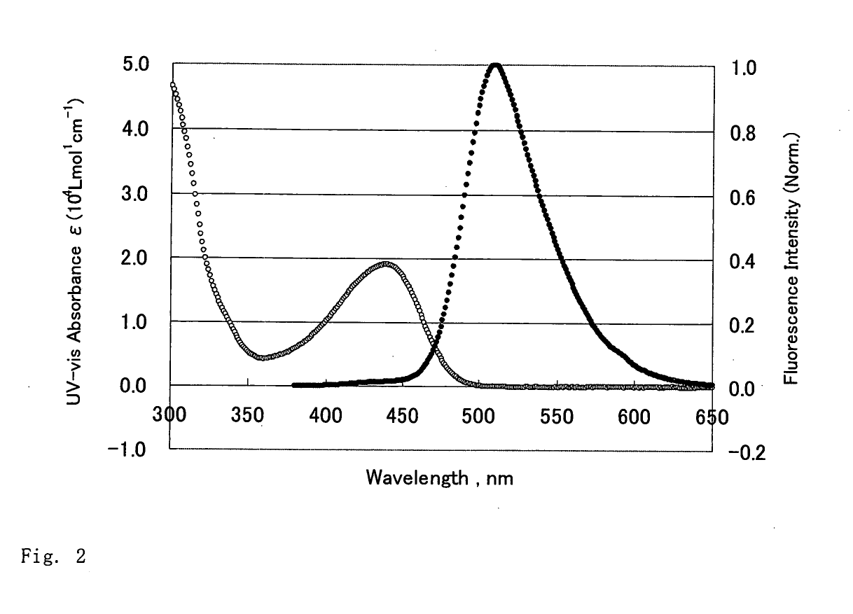

FIG. 3 is a ¹H-NMR spectrum of the dibenzo[c,g]triphenylene derivative obtained in Synthetic Example 1.

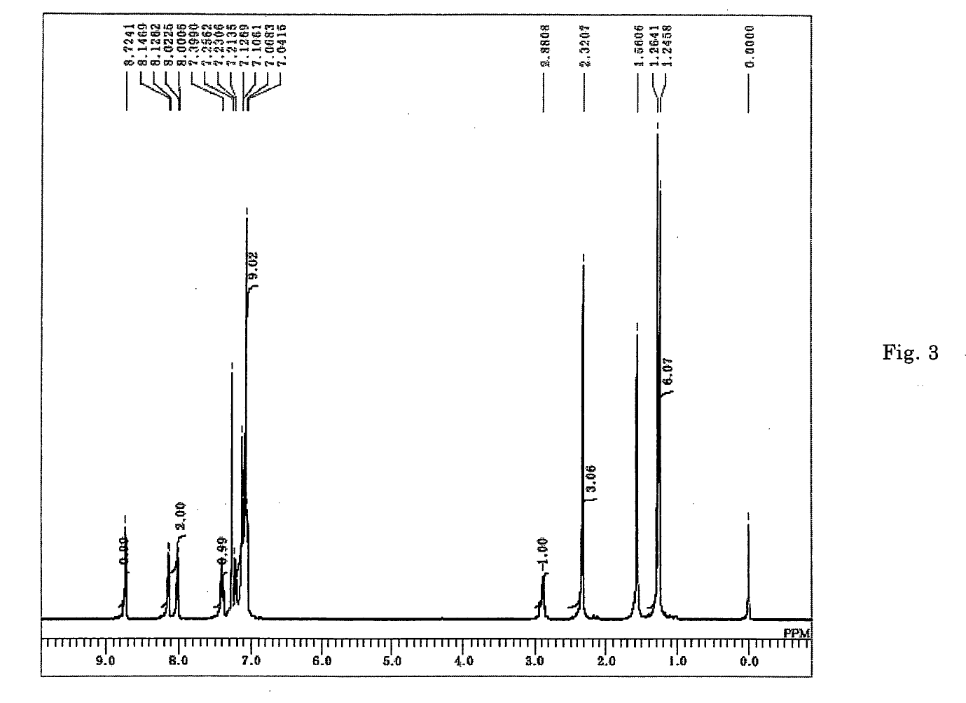

### EXPLANATIONS OF THE CODES

10 Anode
20 Organic compound layer
21 Hole injecting layer
22 Hole transporting layer
23 Light emitting layer
24 Electron transporting layer
25 Electron injecting layer
30 Cathode

### BEST MODE FOR CARRYING OUT THE INVENTION

The dibenzo[c,g]triphenylene derivative of the present invention is represented by the following Formula (1):

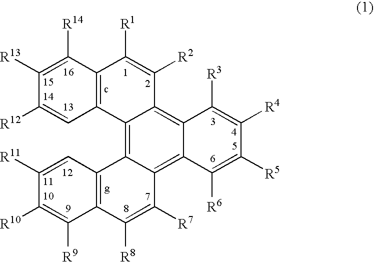

wherein R¹ to R¹⁴ each represent independently a hydrogen atom, a substituted or non-substituted alkyl group having 1 to 50 carbon atoms, a substituted or non-substituted alkoxyalkyl group having 1 to 50 carbon atoms, a substituted or non-substituted aralkyl group having 1 to 50 carbon atoms, a substituted or non-substituted cycloalkyl group having 5 to 50 carbon atoms, a substituted or non-substituted alkoxy group having 1 to 50 carbon atoms, a substituted or non-substituted alkylamino group having 1 to 20 carbon atoms, a substituted or non-substituted alkoxycarbonyl group having 1 to 50 carbon atoms, a substituted or non-substituted aryl group having 5 to 50 carbon atoms, a substituted or non-substituted aryloxy group having 5 to 50 carbon atoms, a substituted or non-substituted arylthio group having 5 to 50 carbon atoms, a substituted or non-substituted arylamino group having 5 to 50 carbon atoms, a substituted or non-substituted heteroaryl group having 5 to 50 carbon atoms, a substituted or non-substituted heteroaryloxy group having 5 to 50 carbon atoms, a substituted or non-substituted heteroarylthio group having 5 to 50 carbon atoms, a substituted or non-substituted heteroarylamino group having 5 to 50 carbon atoms, a substituted or non-substituted silyl group having 1 to 20 carbon atoms, a halogen atom, a cyano group or a nitro group; when all of the groups adjacent to each other out of the groups represented by R¹ to R¹⁴ are not hydrogen groups, they may be combined with each other to form saturated or unsaturated cyclic structures, and these cyclic structures may be substituted.

The specific examples of the respective groups represented by R¹ to R¹⁴ in Formula (1) shall be explained below.

The substituted or non-substituted aryl group having 5 to 50 ring carbon atoms includes phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, p-(2-phenylpropyl)phenyl, 3-methyl-2-naphthyl, 4-methyl-1-naphthyl, 4-methyl-1-anthryl, 4′-methylbiphenylyl, 4″-t-butyl-p-terphenyl-4-yl and the like. It is preferably a substituted or non-substituted aryl group having 6 to 20 ring carbon atoms.

The substituted or non-substituted heteroaryl group having 5 to 50 ring carbon atoms includes 1-pyrrolyl, 2-pyrrolyl, 3-pyrrolyl, pyrazinyl, 2-pyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthryldinyl, 1-acridinyl, 2-acridinyl, 3-acridinyl, 4-acridinyl, 9-acridinyl, 1,7-phenanthroline-2-yl, 1,7-phenanthroline-3-yl, 1,7-phenanthroline-4-yl, 1,7-phenanthroline-5-yl, 1,7-phenanthroline-6-yl, 1,7-phenanthroline-8-yl, 1,7-phenanthroline-9-yl, 1,7-phenanthroline-10-yl, 1,8-phenanthroline-2-yl, 1,8-phenanthroline-3-yl, 1,8-phenanthroline-4-yl, 1,8-phenanthroline-5-yl, 1,8-phenanthroline-6-yl, 1,8-phenanthroline-7-yl, 1,8-phenanthroline-9-yl, 1,8-phenanthroline-10-yl, 1,9-phenanthroline-2-yl, 1,9-phenanthroline-3-yl, 1,9-phenanthroline-4-yl, 1,9-phenanthroline-5-yl, 1,9-phenanthroline-6-yl, 1,9-phenanthroline-7-yl, 1,9-phenanthroline-8-yl, 1,9-phenanthroline-10-yl, 1,10-phenanthroline-2-yl, 1,10-phenanthroline-3-yl, 1,10-phenanthroline-4-yl, 1,10-phenanthroline-5-yl, 2,9-phenanthroline-1-yl, 2,9-phenanthroline-3-yl, 2,9-phenanthroline-4-yl, 2,9-phenanthroline-5-yl, 2,9-phenanthroline-6-yl, 2,9-phenanthroline-7-yl, 2,9-phenanthroline-8-yl, 2,9-phenanthroline-10-yl, 2,8-phenanthroline-1-yl, 2,8-phenanthroline-3-yl, 2,8-phenanthroline-4-yl, 2,8-phenanthroline-5-yl, 2,8-phenanthroline-6-yl, 2,8-phenanthroline-7-yl, 2,8-phenanthroline-9-yl, 2,8-phenanthroline-10-yl, 2,7-phenanthroline-1-yl, 2,7-phenanthroline-3-yl, 2,7-phenanthroline-4-yl, 2,7-phenanthroline-5-yl, 2,7-phenanthroline-6-yl, 2,7-phenanthroline-8-yl, 2,7-phenanthroline-9-yl, 2,7-phenanthroline-10-yl, 1-phenazinyl, 2-phenazinyl, 1-phenothiazinyl, 2-phenothiazinyl, 3-phenothiazinyl, 4-phenothiazinyl, 10-phenothiazinyl, 1-phenoxazinyl, 2-phenoxazinyl, 3-phenoxazinyl, 4-phenoxazinyl, 10-phenoxazinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrole-1-yl, 2-methylpyrrole-3-yl, 2-methylpyrrole-4-yl, 2-methylpyrrole-5-yl, 3-methylpyrrole-1-yl, 3-methylpyrrole-2-yl, 3-methylpyrrole-4-yl, 3-methylpyrrole-5-yl, 2-t-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl and the like. It is preferably a substituted or non-substituted heteroaryl group having 5 to 20 ring carbon atoms.

The substituted or non-substituted alkyl group having 1 to 50 carbon atoms includes methyl, ethyl, propyl, isopropyl, n-butyl, s-butyl, isobutyl, t-butyl, n-pentyl, n-hexyl, n-heptyl, n-octyl, hydroxymethyl, 1-hydroxyethyl, 2-hydroxyethyl, 2-hydroxyisobutyl, 1,2-dihydroxyethyl, 1,3-dihydroxyisopropyl, 2,3-dihydroxy-t-butyl, 1,2,3-trihydroxypropyl, chloromethyl, 1-chloroethyl, 2-chloroethyl, 2-chloroisobutyl, 1,2-dichloroethyl, 1,3-dichloroisopropyl, 2,3-dichloro-t-butyl, 1,2,3-trichloropropyl, bromomethyl, 1-bromoethyl, 2-bromoethyl, 2-bromoisobutyl, 1,2-dibromoethyl, 1,3-dibromoisopropyl, 2,3-dibromo-t-butyl, 1,2,3-tribromopropyl, iodomethyl, 1-iodoethyl, 2-iodoethyl, 2-iodoisobutyl, 1,2-diiodoethyl, 1,3-diiodoisopropyl, 2,3-diiodo-t-butyl, 1,2,3-triiodopropyl, aminomethyl, 1-aminoethyl, 2-aminoethyl, 2-aminoisobutyl, 1,2-diaminoethyl, 1,3-diaminoisopropyl, 2,3-diamino-t-butyl, 1,2,3-triaminopropyl, cyanomethyl, 1-cyanoethyl, 2-cyanoethyl, 2-cyanoisobutyl, 1,2-dicyanoethyl, 1,3-dicyanoisopropyl, 2,3-dicyano-t-butyl, 1,2,3-tricyanopropyl, nitromethyl, 1-nitroethyl, 2-nitroethyl, 2-nitroisobutyl, 1,2-dinitroethyl, 1,3-dinitroisopropyl, 2,3-dinitro-t-butyl, 1,2,3-trinitropropyl and the like. It is preferably a substituted or non-substituted alkyl group having 1 to 20 carbon atoms.

The substituted or non-substituted cycloalkyl group having 1 to 50 carbon atoms includes cyclopentyl, cyclohexyl, 4-methylcyclohexyl, 1-adamantyl, 2-adamantyl, 1-norbornyl, 2-norbornyl and the like. It is preferably a substituted or non-substituted cycloalkyl group having 5 to 20 carbon atoms.

The substituted or non-substituted alkoxy group having 1 to 50 carbon atoms is a group represented by —OY, and Y includes the same examples as given in the substituted or non-substituted alkyl group having 1 to 50 carbon atoms described above. It is preferably a substituted or non-substituted alkoxy group having 1 to 20 carbon atoms.

The substituted or non-substituted aralkyl group having 6 to 50 carbon atoms represented includes benzyl, 1-phenylethyl, 2-phenylethyl, 1-phenylisopropyl, 2-phenylisopropyl, phenyl-t-butyl, α-naphthylmethyl, 1-α-naphthylethyl, 2-α-naphthylethyl, 1-α-naphthylisopropyl, 2-α-naphthylisopropyl, β-naphthylmethyl, 1-β-naphthylethyl, 2-β-naphthylethyl, 1-β-naphthylisopropyl, 2-β-naphthylisopropyl, 1-pyrrolylmethyl, 2-(1-pyrrolyl)ethyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, 1-chloro-2-phenylisopropyl and the like. It is preferably a substituted or non-substituted aralkyl group having 7 to 20 carbon atoms.

The substituted or non-substituted aryloxy group having 5 to 50 ring carbon atoms and the substituted or non-substituted arylthio group having 5 to 50 ring carbon atoms are groups represented respectively by —OY′ and —SY″, and Y′ and Y″ include the same examples as given in the substituted or non-substituted aryl group having 5 to 50 ring carbon atoms described above.

The substituted or non-substituted alkoxycarbonyl group having 2 to 50 carbon atoms is a group represented by —COOZ, and Z includes the same examples as given in the substituted or non-substituted alkyl group having 1 to 50 carbon atoms described above. It is preferably a substituted or non-substituted alkoxycarbonyl group having 1 to 20 carbon atoms.

The substituted or non-substituted aryl group having 5 to 50 ring carbon atoms in the substituted or non-substituted arylamino group having 5 to 50 ring carbon atoms includes the same examples as given in the substituted or non-substituted aryl group having 5 to 50 ring carbon atoms described above. It is preferably an amino group substituted with a substituted or non-substituted aryl group having 5 to 20 ring carbon atoms.

The silyl group which is the substituent includes trimethylsilyl, triethylsilyl, t-butyldimethylsilyl, vinyldimethylsilyl, propyldimethylsilyl, triphenylsilyl and the like.

The halogen atom includes a fluorine atom, a chlorine atom, a bromine atom and an iodine atom.

Substituents in the groups represented by R¹ to R⁴ described above include a halogen atom, a hydroxy group, a nitro group, a cyano group, a silyl group, an alkyl group, an aryl group, a cycloalkyl group, an alkoxy group, an aromatic heteroaryl group, an aralkyl group, an aryloxy group, an arylthio group, an alkoxycarbonyl group or a carboxy group.

In R¹ to R⁴, the saturated or unsaturated cyclic structures formed by allowing the adjacent groups to be combined with each other are preferably five-membered or six-membered rings, and these cyclic structures may be substituted.

The specific examples of the dibenzo[c,g]triphenylene derivative of the present invention represented by Formula (1) are shown below but shall not be restricted to these compounds given as the examples.

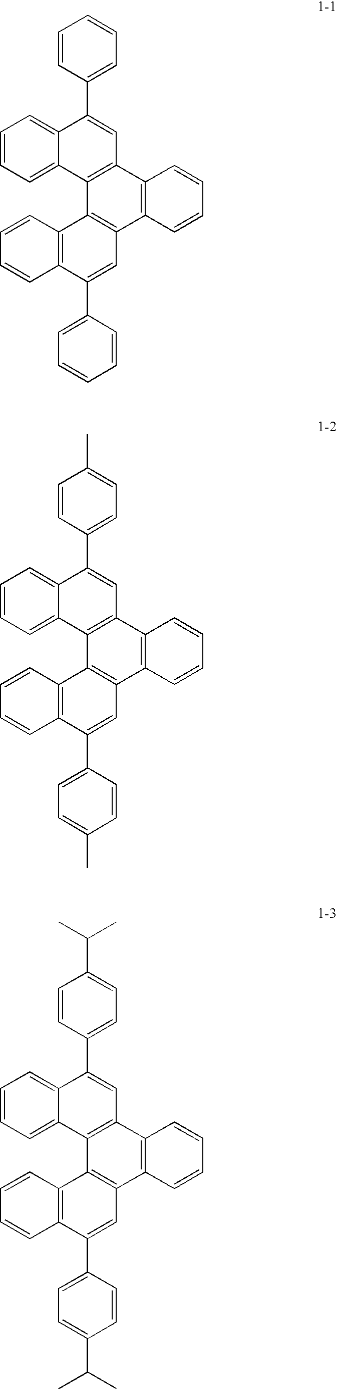

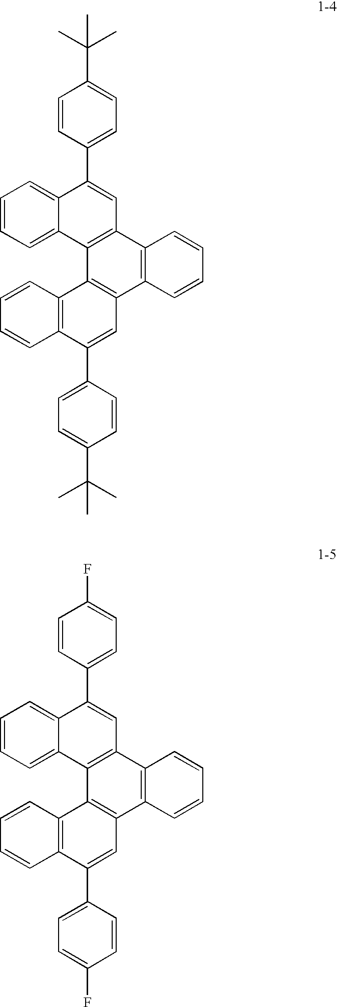

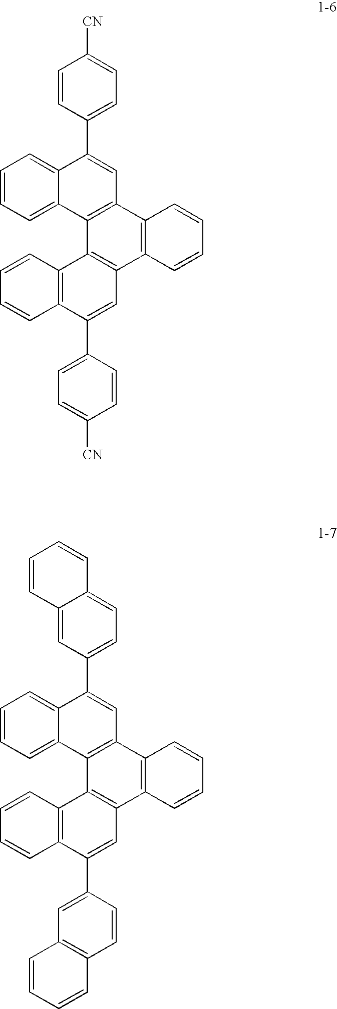

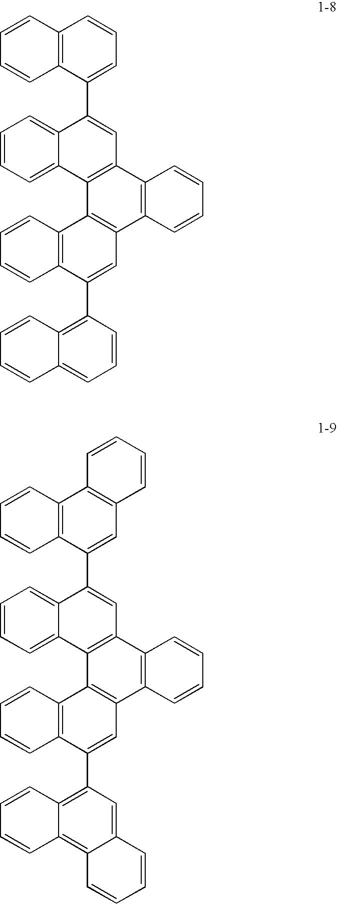

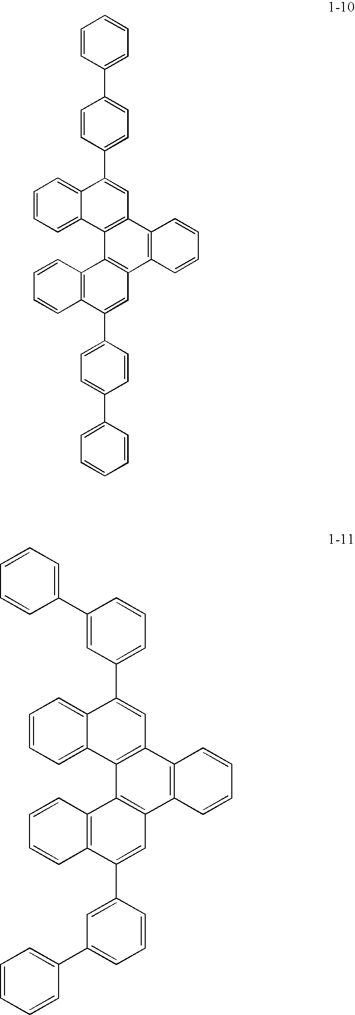

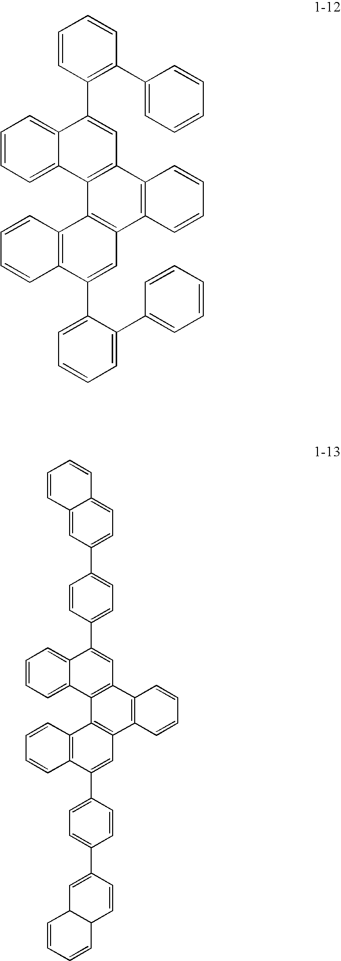

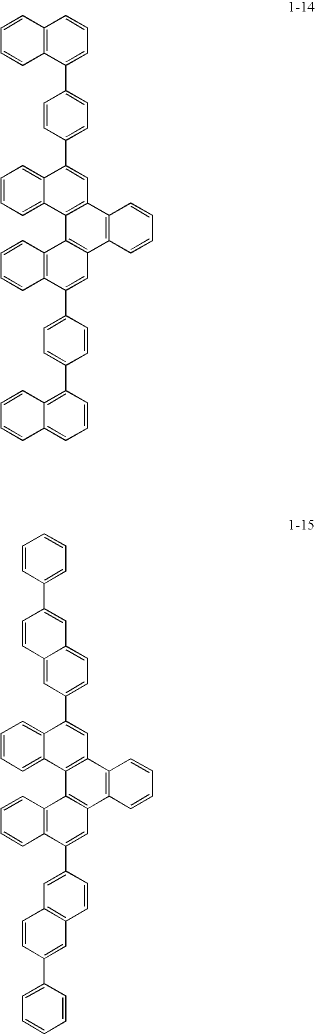

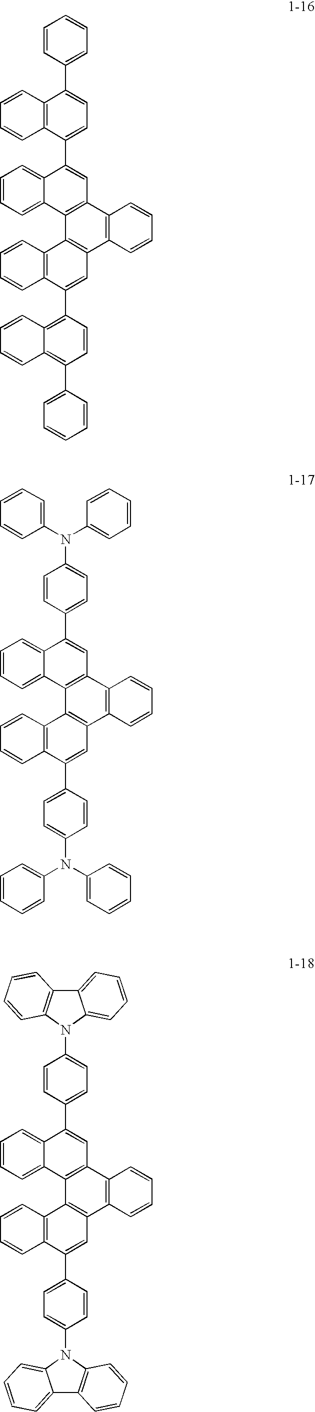

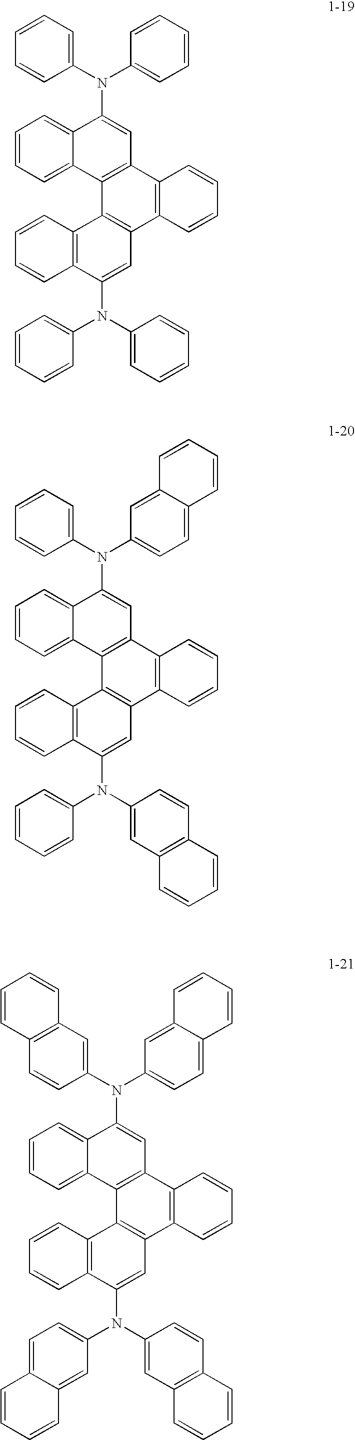

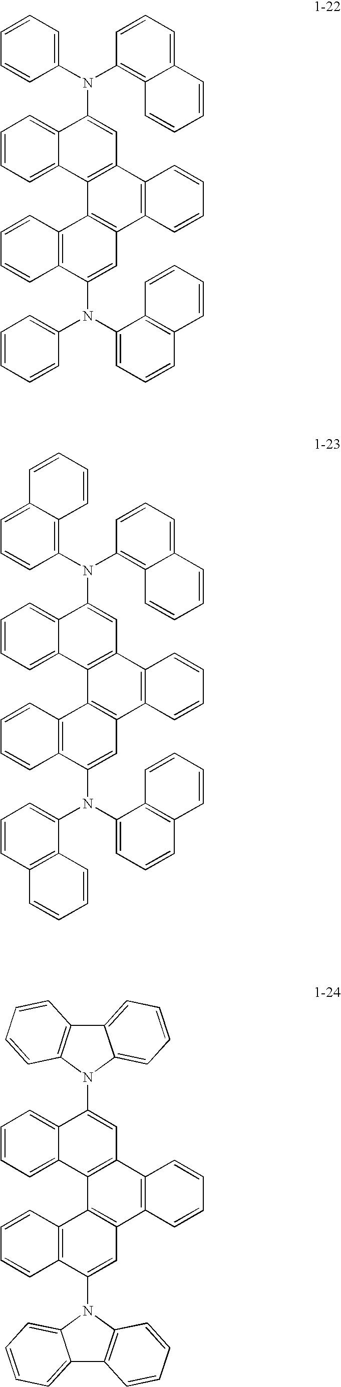

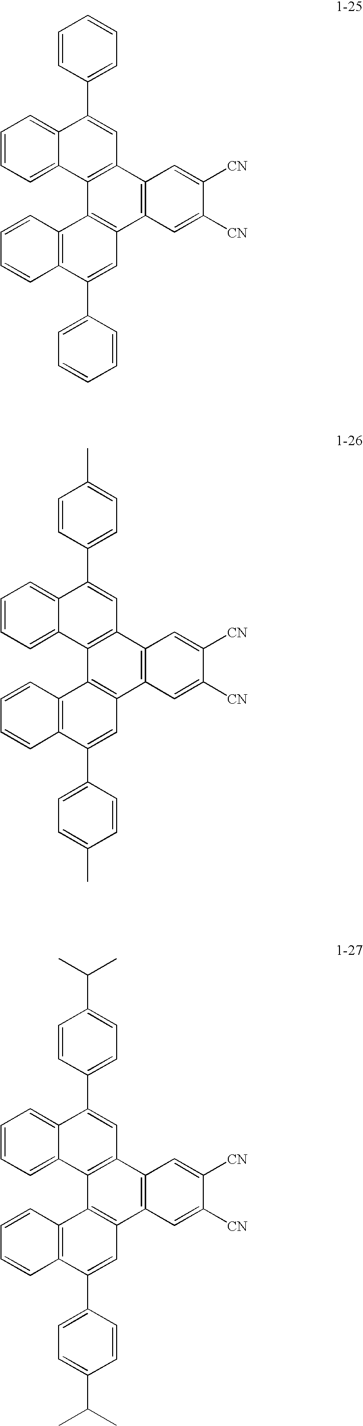

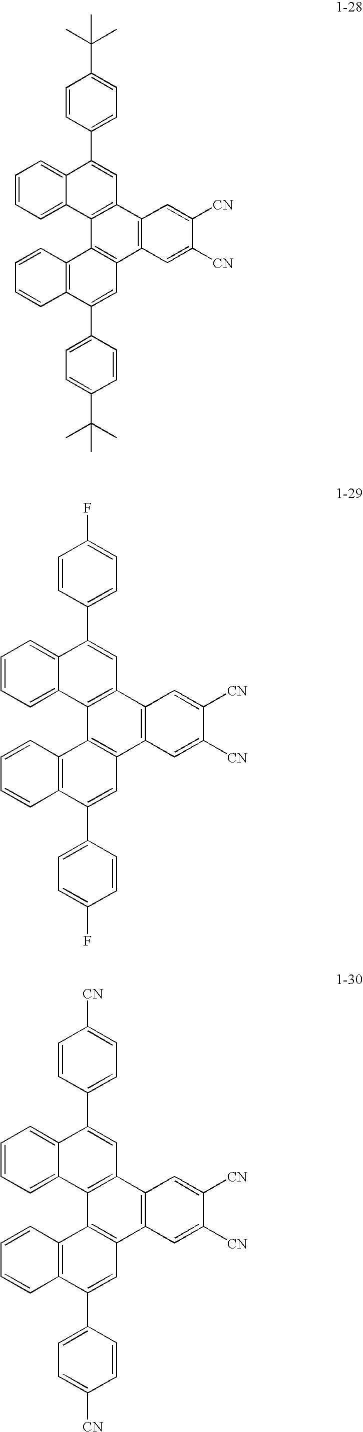

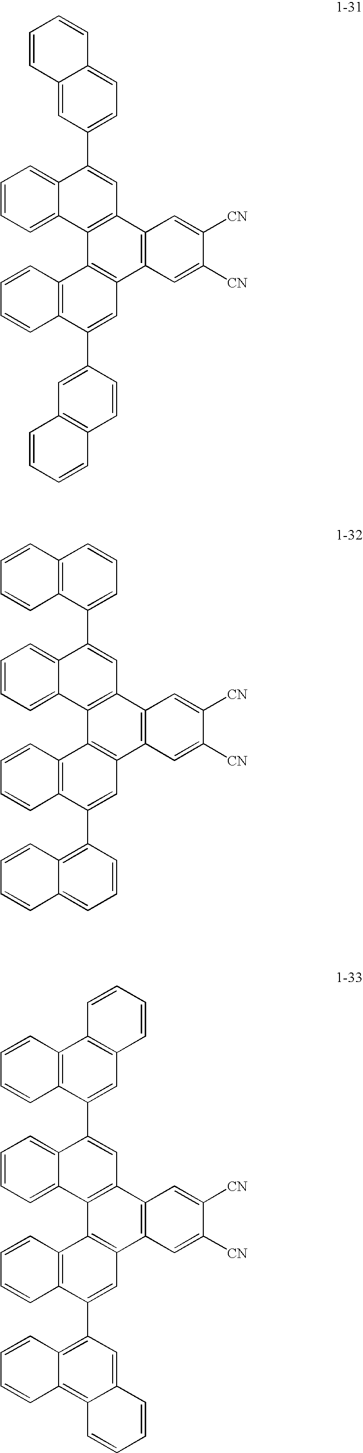

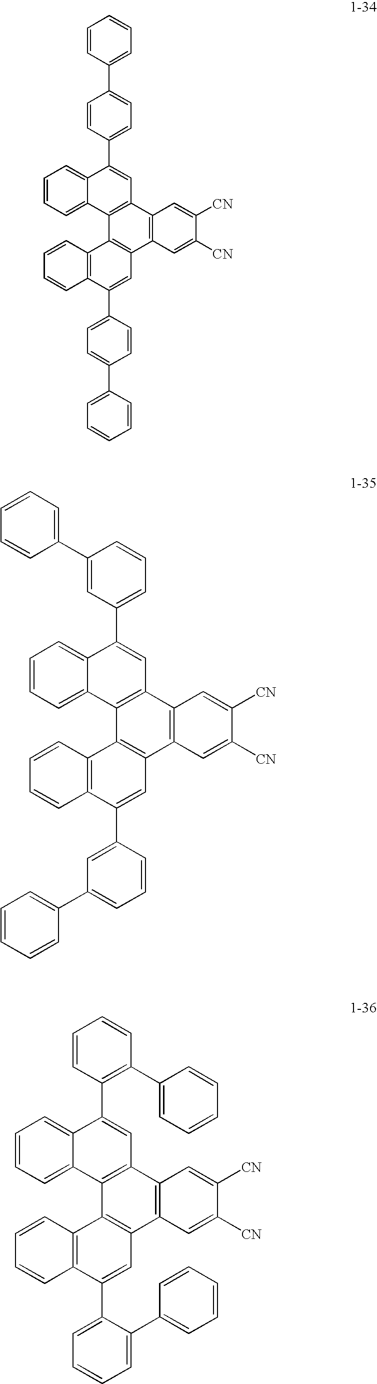

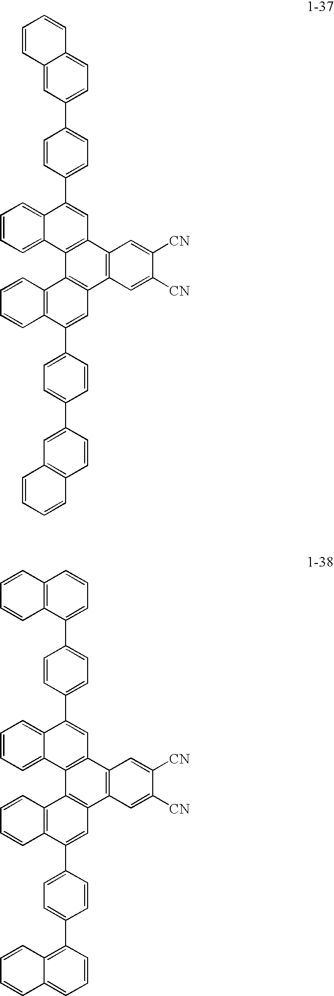

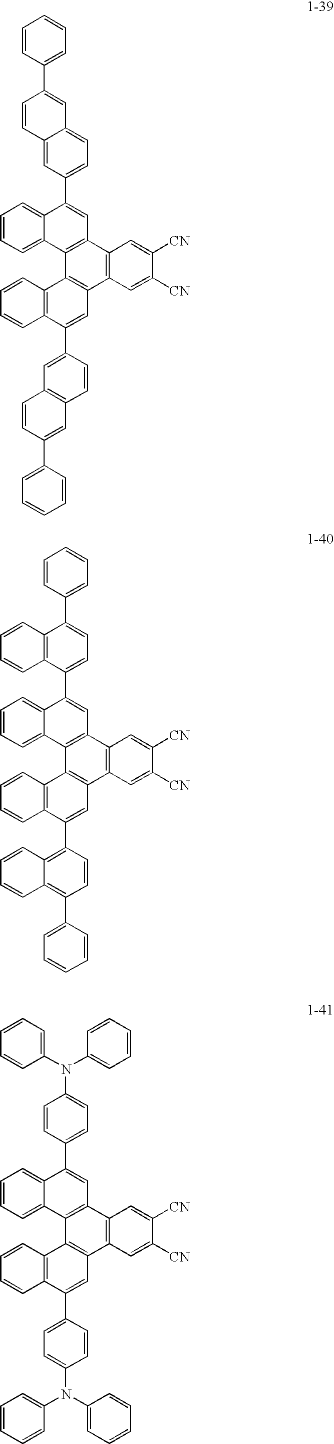

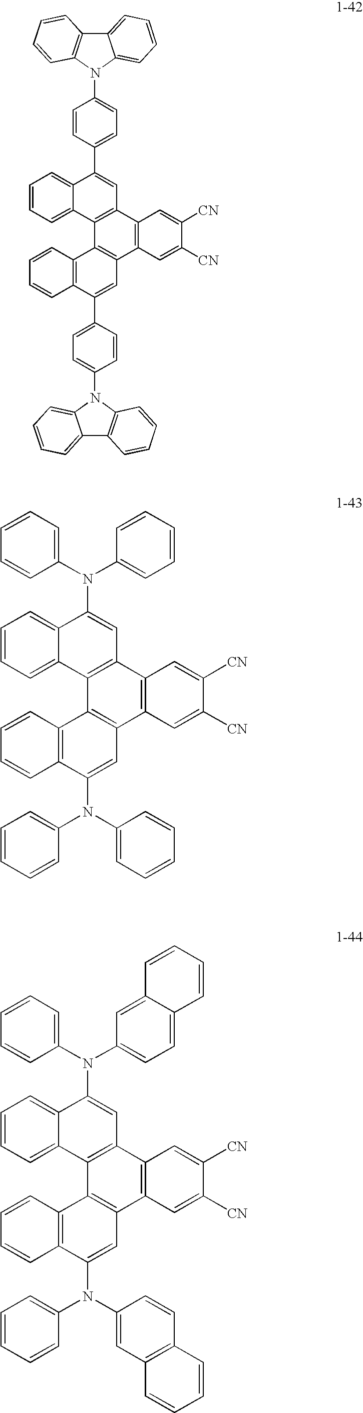

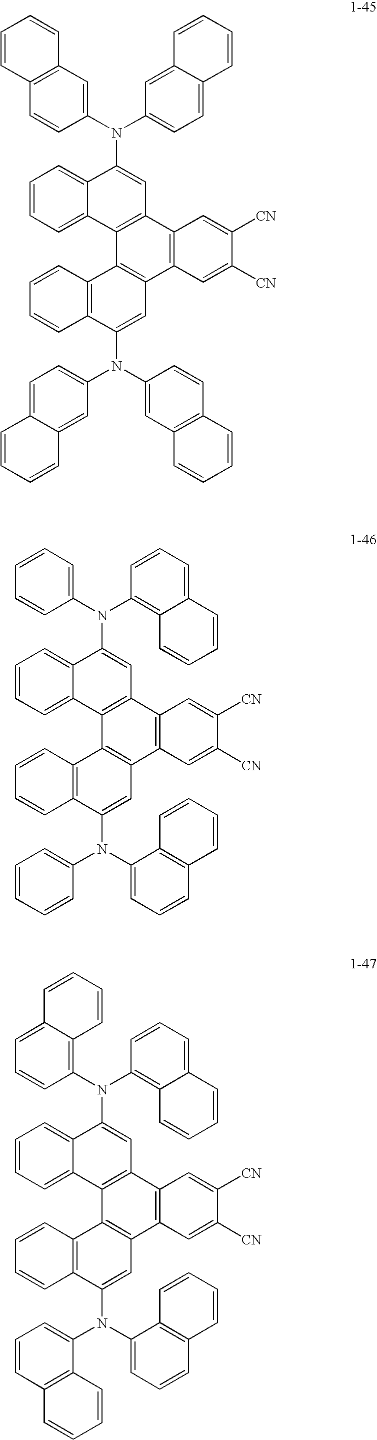

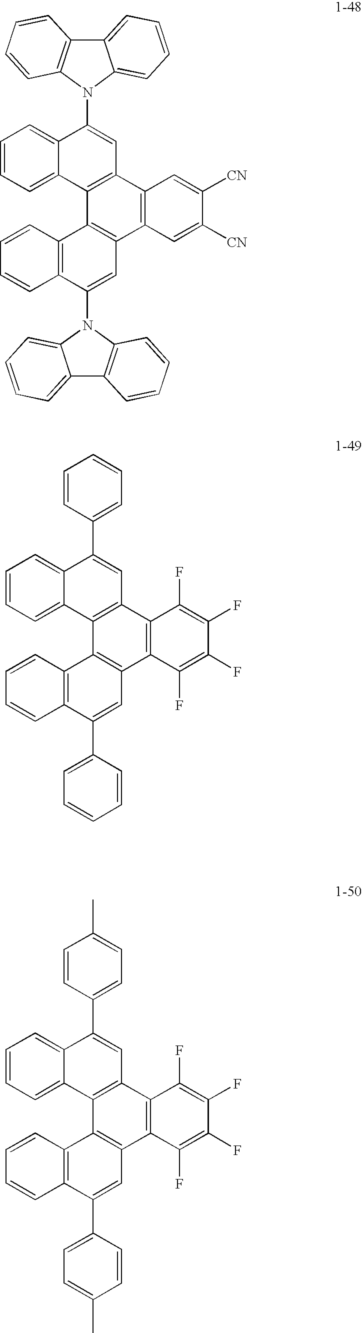

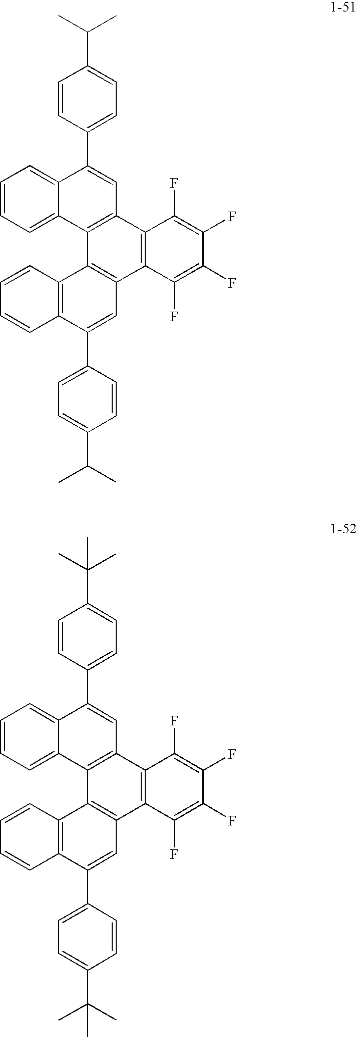

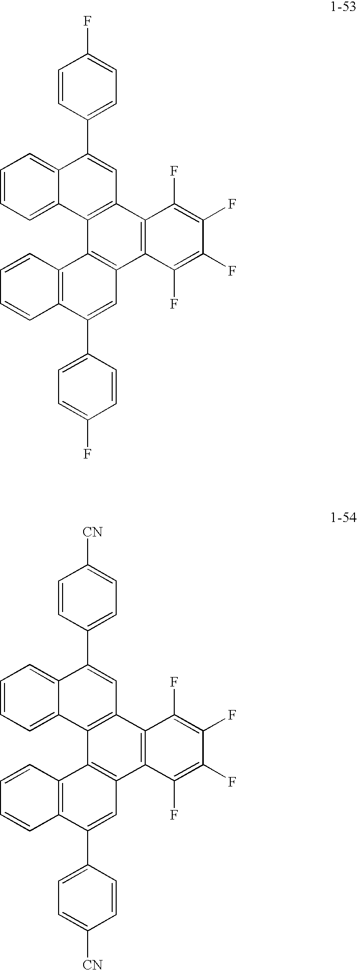

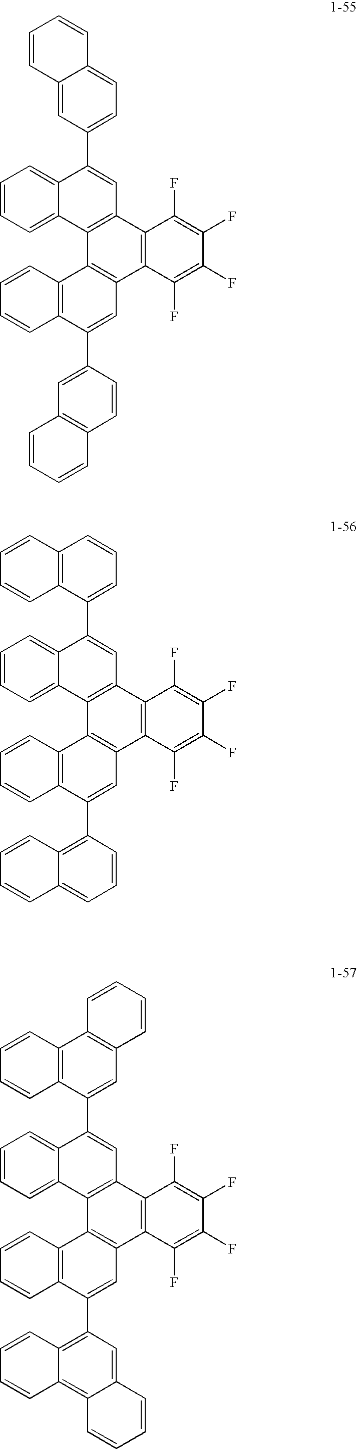

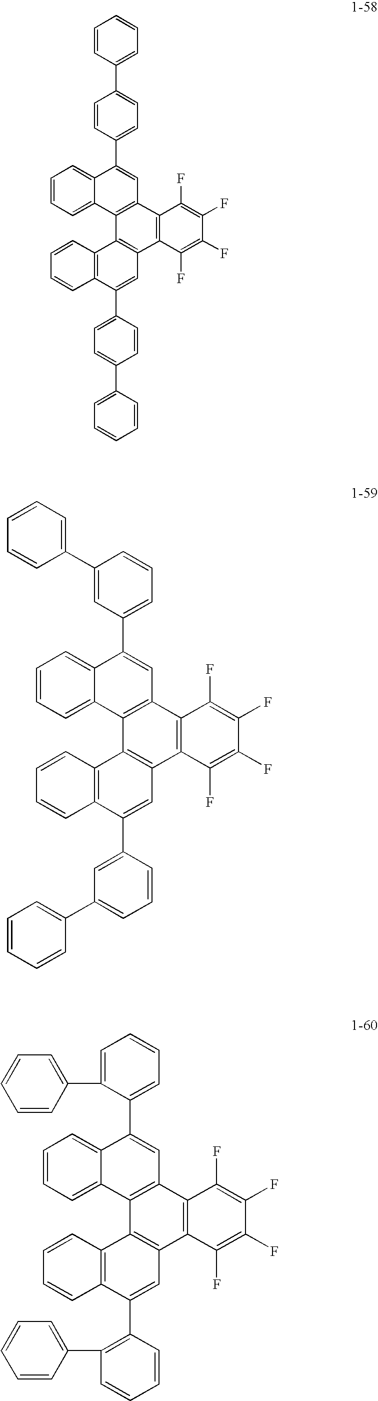

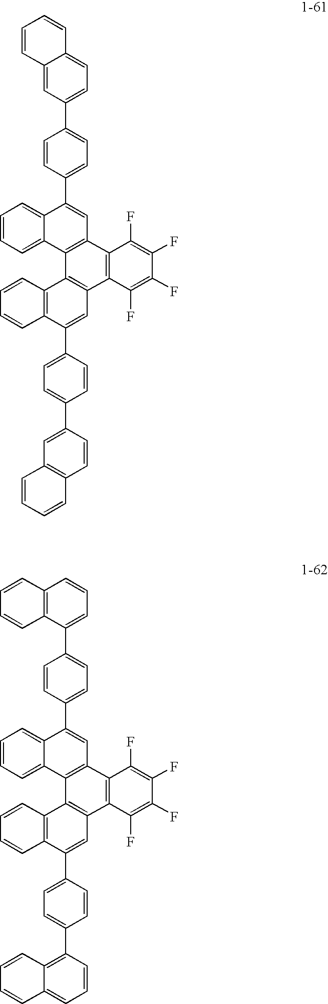

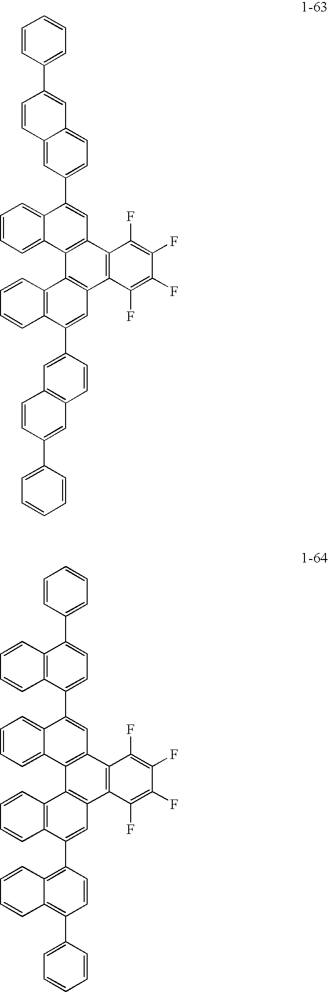

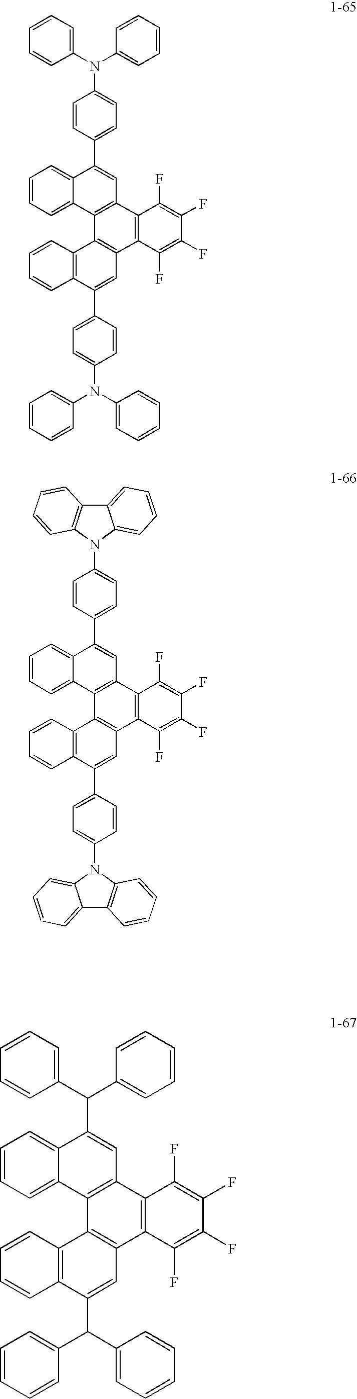

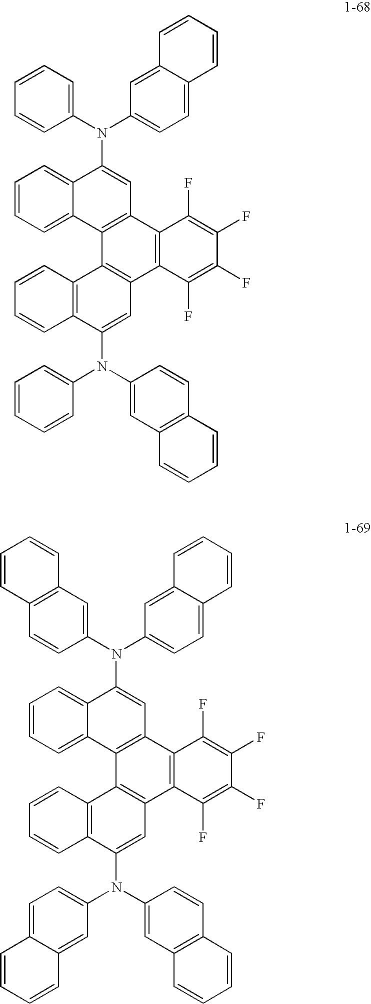

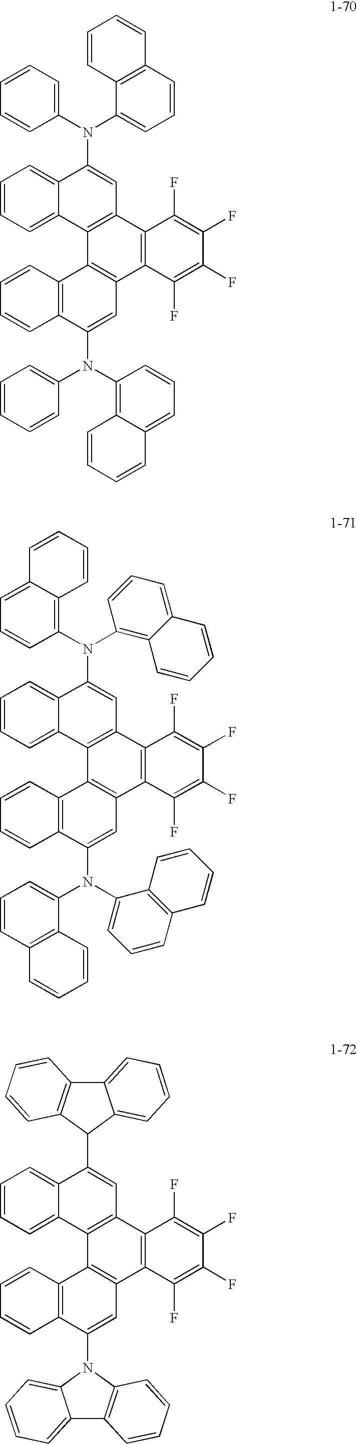

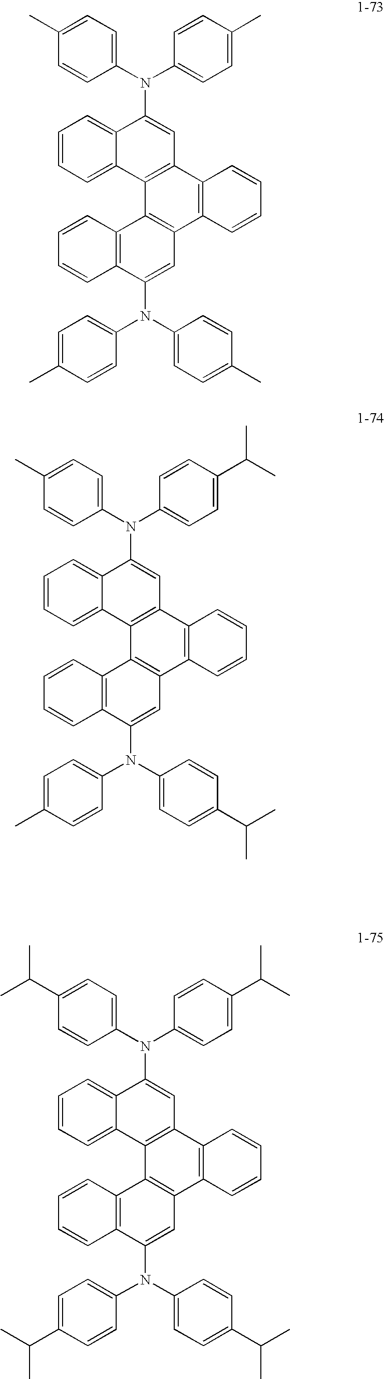

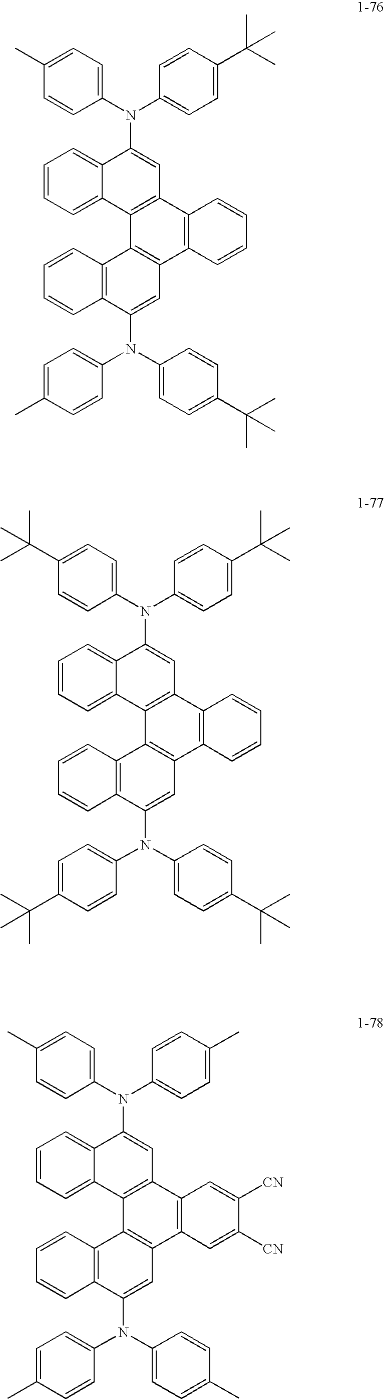

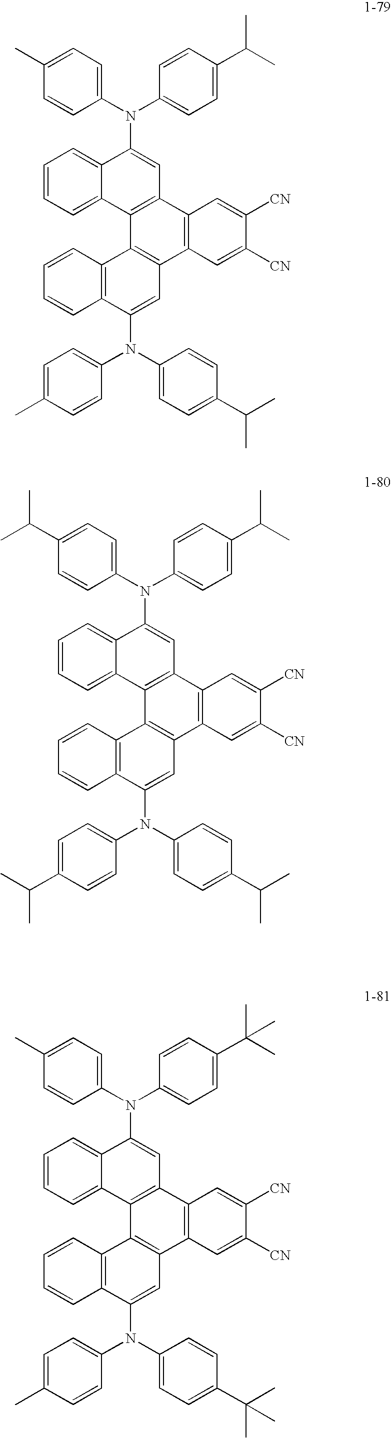

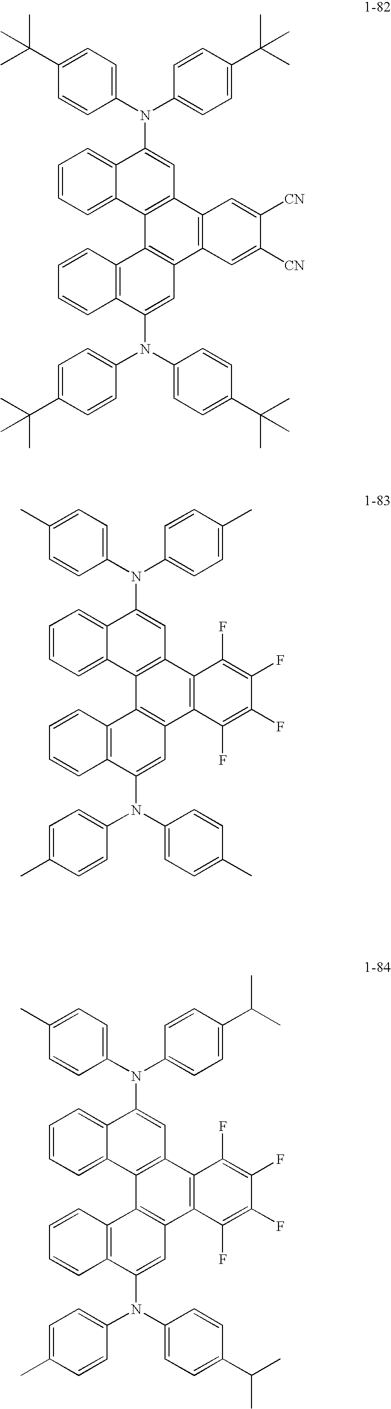

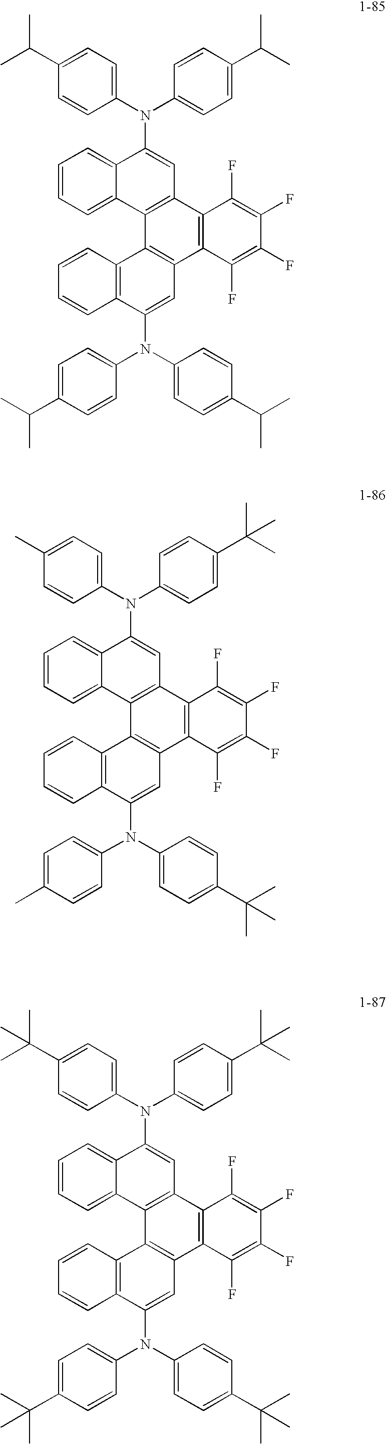

Dibenzo[c,g]triphenylene precursors suitably used in the present invention can be synthesized by any of processes described in the following documents.

### Warren E. Piers et al., Journal of Organic Chemistry, Vol. 70, 5309 (2005)

### Braja K, Mandal et al., Journal of Organic Chemistry, Vol. 68, 652 (2003)

### Timothy M, Swager et al., Journal of American Chemical Society, Vol. 119, 4578 (1997)

Tinnemans, A. H. A. et al., Recl. Trav. Chim. Pays-Bas, Vol. 94, 239 (1975)

The dibenzo[c,g]triphenylene precursor thus obtained is subjected subsequently to a Suzuki cross coupling reaction or a Buchwald-Hartwig cross coupling reaction using a palladium catalyst, whereby the dibenzo[c,g]triphenylene derivative of the present invention can be produced.

The dibenzo[c,g]triphenylene derivative of the present invention is preferably used as a material for an organic EL device, and it is more preferably used as a light emitting material for an organic EL device.

In the organic EL device of the present invention in which an organic compound layer comprising a single layer or plural layers including at least a light emitting layer is interposed between a pair of electrodes, at least one layer in the above organic compound layer contains at least one kind of the dibenzo[c,g]triphenylene derivatives of the present invention.

In the organic EL device of the present invention, the light emitting layer described above preferably contains at least one kind of the dibenzo[c,g]triphenylene derivatives described above, and the dibenzo[c,g]triphenylene derivative of the present invention is contained in the light emitting layer in a proportion of preferably 0.01 to 20% by weight, more preferably 0.5 to 20% by weight.

When the dibenzo[c,g]triphenylene derivative of the present invention is used as the light emitting material for the organic EL device, the light emitting layer described above preferably contains at least one kind of the dibenzo[c,g]triphenylene derivatives described above and at least one selected from compounds represented by the following Formulas (2a) to (2d), and at least one selected from the compounds represented by the following Formulas (2a) to (2d) is preferably a host material.

Formulas (2a) to (2d) shall be explained below.

### Formula (2a)

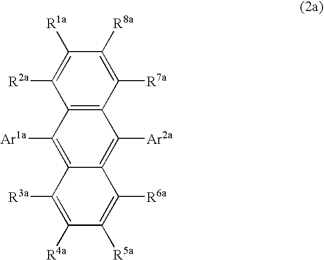

(in Formula (2a), Ar^{1a} and Ar^{2a} each are independently a group derived from a substituted or non-substituted aromatic ring having 6 to 20 ring carbon atoms; the aromatic ring described above may be substituted with at least one substituent; the substituent described above is selected from a substituted or non-substituted aryl group having 6 to 50 ring carbon atoms, a substituted or non-substituted alkyl group having 1 to 50 carbon atoms, a substituted or non-substituted cycloalkyl group having 3 to 50 carbon atoms, a substituted or non-substituted alkoxy group having 1 to 50 carbon atoms, a substituted or non-substituted aralkyl group having 6 to 50 carbon atoms, a substituted or non-substituted aryloxy group having 5 to 50 ring carbon atoms, a substituted or non-substituted arylthio group having 5 to 50 ring carbon atoms, a substituted or non-substituted alkoxycarbonyl group having 1 to 50 carbon atoms, a substituted or non-substituted silyl group, a carboxy group, a halogen atom, a cyano group, a nitro group and a hydroxy group; when the aromatic ring described above is substituted with two or more substituents, the substituents described above may be the same or different, and the adjacent substituents may be combined with each other to form a saturated or unsaturated cyclic structure;
R^{1a} to R^{8a} each are selected independently from a hydrogen atom, a substituted or non-substituted aryl group having 6 to 50 ring carbon atoms, a substituted or non-substituted heteroaryl group having 5 to 50 ring carbon atoms, a substituted or non-substituted alkyl group having 1 to 50 carbon atoms, a substituted or non-substituted cycloalkyl group having 3 to 50 carbon atoms, a substituted or non-substituted alkoxy group having 1 to 50 carbon atoms, a substituted or non-substituted aralkyl group having 6 to 50 carbon atoms, a substituted or non-substituted aryloxy group having 5 to 50 ring carbon atoms, a substituted or non-substituted arylthio group having 5 to 50 ring carbon atoms, a substituted or non-substituted alkoxycarbonyl group having 1 to 50 carbon atoms, a substituted or non-substituted silyl group, a carboxy group, a halogen atom, a cyano group, a nitro group and a hydroxy group).

The group derived from the substituted or non-substituted aromatic ring having 6 to 20 ring carbon atoms represented by Ar^{1a} and Ar^{2a} in Formula (2a) includes phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, p-(2-phenylpropyl)phenyl, 3-methyl-2-naphthyl, 4-methyl-1-naphthyl, 4-methyl-1-anthryl, 4′-methylbiphenylyl, 4″-t-butyl-p-terphenyl-4-yl and the like. It is preferably a group derived from a substituted or non-substituted aromatic ring having 10 to 14 ring carbon atoms, and it is particularly 1-naphthyl, 2-naphthyl or 9-phenanthryl.

The substituted or non-substituted aryl group having 6 to 50 ring carbon atoms in the aromatic ring described above includes phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, p-(2-phenylpropyl)phenyl, 3-methyl-2-naphthyl, 4-methyl-1-naphthyl, 4-methyl-1-anthryl, 4′-methylbiphenylyl, 4″-t-butyl-p-terphenyl-4-yl and the like. It is preferably a substituted or non-substituted aryl group having 6 to 18 ring carbon atoms, and it is particularly phenyl, 1-naphthyl, 2-naphthyl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, o-tolyl, m-tolyl, p-tolyl or p-t-butylphenyl.

The substituted or non-substituted aryl group having 6 to 50 ring carbon atoms represented by R^{1a} to R^{8a} in Formula (2a) includes phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, p-(2-phenylpropyl)phenyl, 3-methyl-2-naphthyl, 4-methyl-1-naphthyl, 4-methyl-1-anthryl, 4′-methylbiphenylyl, 4″-t-butyl-p-terphenyl-4-yl and the like.

The substituted or non-substituted heteroaryl group having 5 to 50 ring carbon atoms represented by R^{1a} to R^{8a} in Formula (2a) includes 1-pyrrolyl, 2-pyrrolyl, 3-pyrrolyl, pyrazinyl, 2-pyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthryldinyl, 1-acridinyl, 2-acridinyl, 3-acridinyl, 4-acridinyl, 9-acridinyl, 1,7-phenanthroline-2-yl, 1,7-phenanthroline-3-yl, 1,7-phenanthroline-4-yl, 1,7-phenanthroline-5-yl, 1,7-phenanthroline-6-yl, 1,7-phenanthroline-8-yl, 1,7-phenanthroline-9-yl, 1,7-phenanthroline-10-yl, 1,8-phenanthroline-2-yl, 1,8-phenanthroline-3-yl, 1,8-phenanthroline-4-yl, 1,8-phenanthroline-5-yl, 1,8-phenanthroline-6-yl, 1,8-phenanthroline-7-yl, 1,8-phenanthroline-9-yl, 1,8-phenanthroline-10-yl, 1,9-phenanthroline-2-yl, 1,9-phenanthroline-3-yl, 1,9-phenanthroline-4-yl, 1,9-phenanthroline-5-yl, 1,9-phenanthroline-6-yl, 1,9-phenanthroline-7-yl, 1,9-phenanthroline-8-yl, 1,9-phenanthroline-10-yl, 1,10-phenanthroline-2-yl, 1,10-phenanthroline-3-yl, 1,10-phenanthroline-4-yl, 1,10-phenanthroline-5-yl, 2,9-phenanthroline-1-yl, 2,9-phenanthroline-3-yl, 2,9-phenanthroline-4-yl, 2,9-phenanthroline-5-yl, 2,9-phenanthroline-6-yl, 2,9-phenanthroline-7-yl, 2,9-phenanthroline-8-yl, 2,9-phenanthroline-10-yl, 2,8-phenanthroline-1-yl, 2,8-phenanthroline-3-yl, 2,8-phenanthroline-4-yl, 2,8-phenanthroline-5-yl, 2,8-phenanthroline-6-yl, 2,8-phenanthroline-7-yl, 2,8-phenanthroline-9-yl, 2,8-phenanthroline-10-yl, 2,7-phenanthroline-1-yl, 2,7-phenanthroline-3-yl, 2,7-phenanthroline-4-yl, 2,7-phenanthroline-5-yl, 2,7-phenanthroline-6-yl, 2,7-phenanthroline-8-yl, 2,7-phenanthroline-9-yl, 2,7-phenanthroline-10-yl, 1-phenazinyl, 2-phenazinyl, 1-phenothiazinyl, 2-phenothiazinyl, 3-phenothiazinyl, 4-phenothiazinyl, 10-phenothiazinyl, 1-phenoxazinyl, 2-phenoxazinyl, 3-phenoxazinyl, 4-phenoxazinyl, 10-phenoxazinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrole-1-yl, 2-methylpyrrole-3-yl, 2-methylpyrrole-4-yl, 2-methylpyrrole-5-yl, 3-methylpyrrole-1-yl, 3-methylpyrrole-2-yl, 3-methylpyrrole-4-yl, 3-methylpyrrole-5-yl, 2-t-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl and the like.

The substituted or non-substituted alkyl group having 1 to 50 ring carbon atoms in R^{1a} to R^{8a} and the aromatic ring described above in Formula (2a) includes methyl, ethyl, propyl, isopropyl, n-butyl, s-butyl, isobutyl, t-butyl, n-pentyl, n-hexyl, n-heptyl, n-octyl, hydroxymethyl, 1-hydroxyethyl, 2-hydroxyethyl, 2-hydroxyisobutyl, 1,2-dihydroxyethyl, 1,3-dihydroxyisopropyl, 2,3-dihydroxy-t-butyl, 1,2,3-trihydroxypropyl, chloromethyl, 1-chloroethyl, 2-chloroethyl, 2-chloroisobutyl, 1,2-dichloroethyl, 1,3-dichloroisopropyl, 2,3-dichloro-t-butyl, 1,2,3-trichloropropyl, bromomethyl, 1-bromoethyl, 2-bromoethyl, 2-bromoisobutyl, 1,2-dibromoethyl, 1,3-dibromoisopropyl, 2,3-dibromo-t-butyl, 1,2,3-tribromopropyl, iodomethyl, 1-iodoethyl, 2-iodoethyl, 2-iodoisobutyl, 1,2-diiodoethyl, 1,3-diiodoisopropyl, 2,3-diiodo-t-butyl, 1,2,3-triiodopropyl, aminomethyl, 1-aminoethyl, 2-aminoethyl, 2-aminoisobutyl, 1,2-diaminoethyl, 1,3-diaminoisopropyl, 2,3-diamino-t-butyl, 1,2,3-triaminopropyl, cyanomethyl, 1-cyanoethyl, 2-cyanoethyl, 2-cyanoisobutyl, 1,2-dicyanoethyl, 1,3-dicyanoisopropyl, 2,3-dicyano-t-butyl, 1,2,3-tricyanopropyl, nitromethyl, 1-nitroethyl, 2-nitroethyl, 2-nitroisobutyl, 1,2-dinitroethyl, 1,3-dinitroisopropyl, 2,3-dinitro-t-butyl, 1,2,3-trinitropropyl and the like.

The substituted or non-substituted cycloalkyl group having 3 to 50 carbon atoms in R^{1a} to R^{8a} and the aromatic ring described above in Formula (2a) includes cyclopropyl, cyclobutyl, cyclopentyl, cyclohexyl, 4-methylcyclohexyl, 1-adamantyl, 2-adamantyl, 1-norbornyl, 2-norbornyl and the like.

The substituted or non-substituted alkoxy group having 1 to 50 carbon atoms in R^{1a} to R^{8a} and the aromatic ring described above in Formula (2a) is a group represented by —OY, and Y includes the same examples as given in the substituted or non-substituted alkyl group having 1 to 50 carbon atoms in R^{1a} to R^{8a} and the aromatic ring described above.

The substituted or non-substituted aralkyl group having 6 to 50 carbon atoms in R^{1a} to R^{8a} and the aromatic ring described above in Formula (2a) includes benzyl, 1-phenylethyl, 2-phenylethyl, 1-phenylisopropyl, 2-phenylisopropyl, phenyl-t-butyl, α-naphthylmethyl, 1-α-naphthylethyl, 2-α-naphthylethyl, 1-α-naphthylisopropyl, 2-α-naphthylisopropyl, β-naphthylmethyl, 1-β-naphthylethyl, 2-β-naphthylethyl, 1-β-naphthylisopropyl, 2-β-naphthylisopropyl, 1-pyrrolylmethyl, 2-(1-pyrrolyl)ethyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, 1-chloro-2-phenylisopropyl and the like.

The substituted or non-substituted aryloxy group and arylthio group each having 5 to 50 ring carbon atoms in R^{1a} to R^{8a} and the aromatic ring described above in Formula (2a) are represented by —OY′ and SY″ respectively, and Y′ and Y″ include the same examples as given in the substituted or non-substituted aryl group having 6 to 50 ring carbon atoms in R^{1a} to R^{8a} and the aromatic ring described above.

The substituted or non-substituted alkoxycarbonyl group having 1 to 50 carbon atoms in R^{1a} to R^{8a} and the aromatic ring described above in Formula (2a) is represented by —COOZ, and Z includes the same examples as given in the substituted or non-substituted alkyl group having 1 to 50 carbon atoms in R^{1a} to R^{8a} and the aromatic ring described above.

The silyl group which is represented by R^{1a} to R^{8a} in Formula (2a) and which is the substituent of the aromatic ring described above includes trimethylsilyl, triethylsilyl, t-butyldimethylsilyl, vinyldimethylsilyl, propyldimethylsilyl, triphenylsilyl and the like.

The halogen atom which is represented by R^{1a} to R^{8a} in Formula (2a) and which is the substituent of the aromatic ring described above includes fluorine, chlorine, bromine and iodine.

Substituents in the groups represented by R^{1a} to R^{8a} described above and the substituents of the aromatic ring described above include a halogen atom, a hydroxy group, a nitro group, a cyano group, an alkyl group, an aryl group, a cycloalkyl group, an alkoxy group, an aromatic heteroaryl group, an aralkyl group, an aryloxy group, an arylthio group, an alkoxycarbonyl group, a carboxy group and the like.

Further, the cyclic structure which may be formed includes cycloalkanes having 4 to 12 carbon atoms such as cyclobutane, cyclopentane, cyclohexane, adamantane, norbornane and the like, cycloalkenes having 4 to 12 carbon atoms such as cyclobutene, cyclopentene, cyclohexene, cycloheptene, cyclooctene and the like, cycloalkadienes having 6 to 12 carbon atoms such as cyclohexadiene, cycloheptadiene, cyclooctadiene and the like and aromatic rings having 6 to 50 carbon atoms such as benzene, naphthalene, phenanthrene, anthracene, pyrene chrysene, acenaphthylene and the like. Substituents therefor include the same groups as the examples described above.

The anthracene derivative represented by Formula (2a) described above is preferably a compound having a structure represented by the following Formula (2a′):

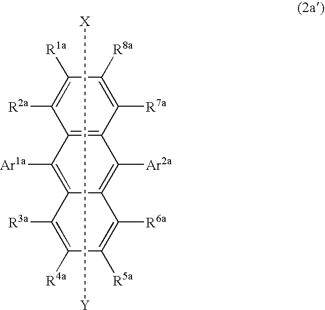

(in Formula (2a′), Ar^{1a}, Ar^{2a}, R^{1a} to R^{8a} each are independently the same as in Formula (2a), and the same specific examples are given;
provided that included is no case where in Formula (2a′), groups staying in a symmetric form with respect to an X-Y axis shown on the anthracene are bonded to a 9-position and a 10-position of the anthracene in the center).

The specific examples of the anthracene derivative represented by Formula (2a) used for the organic EL device of the present invention include publicly known various derivatives such as compounds having two anthracene skeletons in a molecule which are shown in the paragraphs of [0043] to of Japanese Patent Application Laid-Open No. 356033/2004 and compounds having one anthracene skeleton which are shown at pages 27 to 28 of International Publication No. 2005/061656. The representative specific examples thereof are shown below.

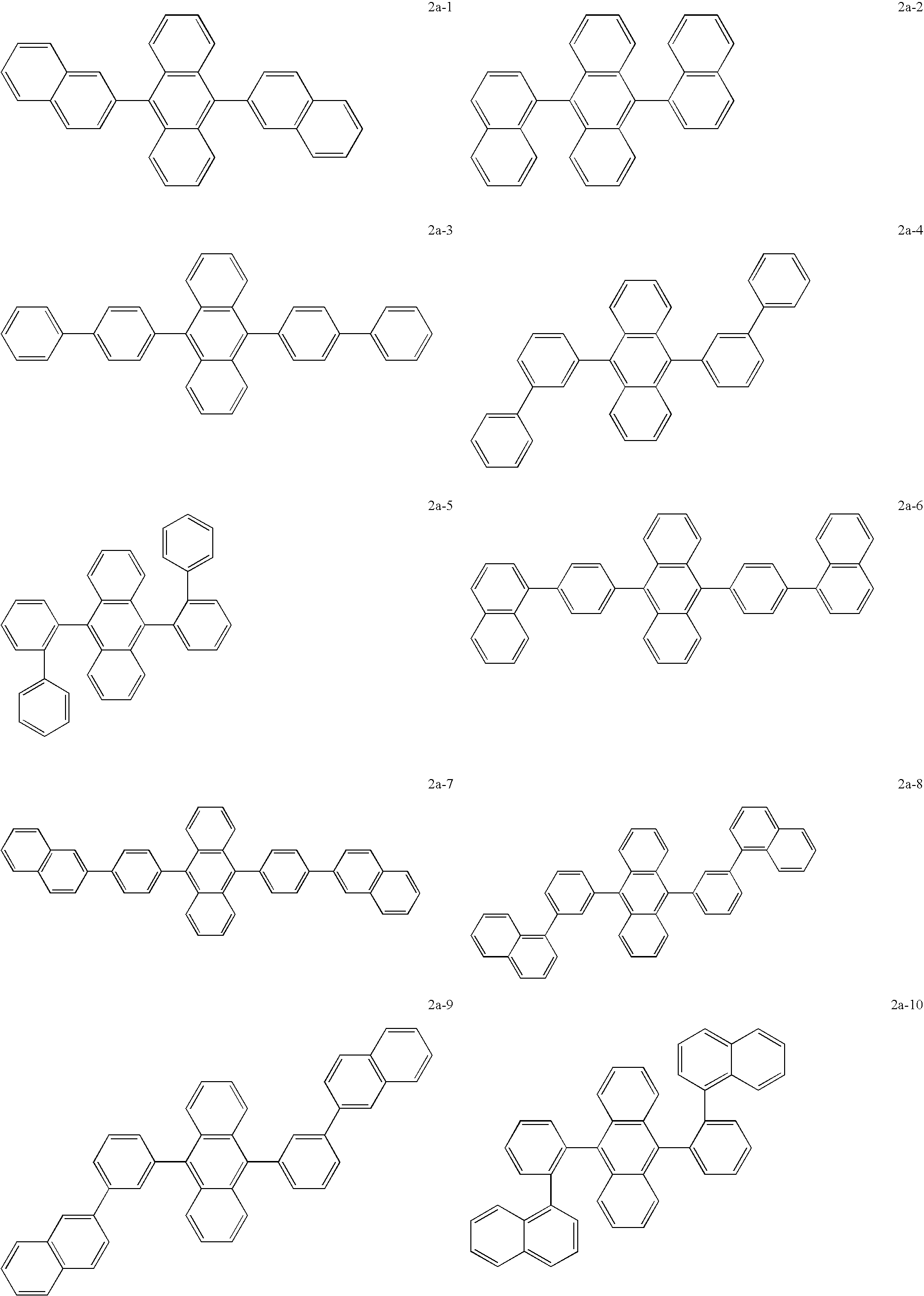

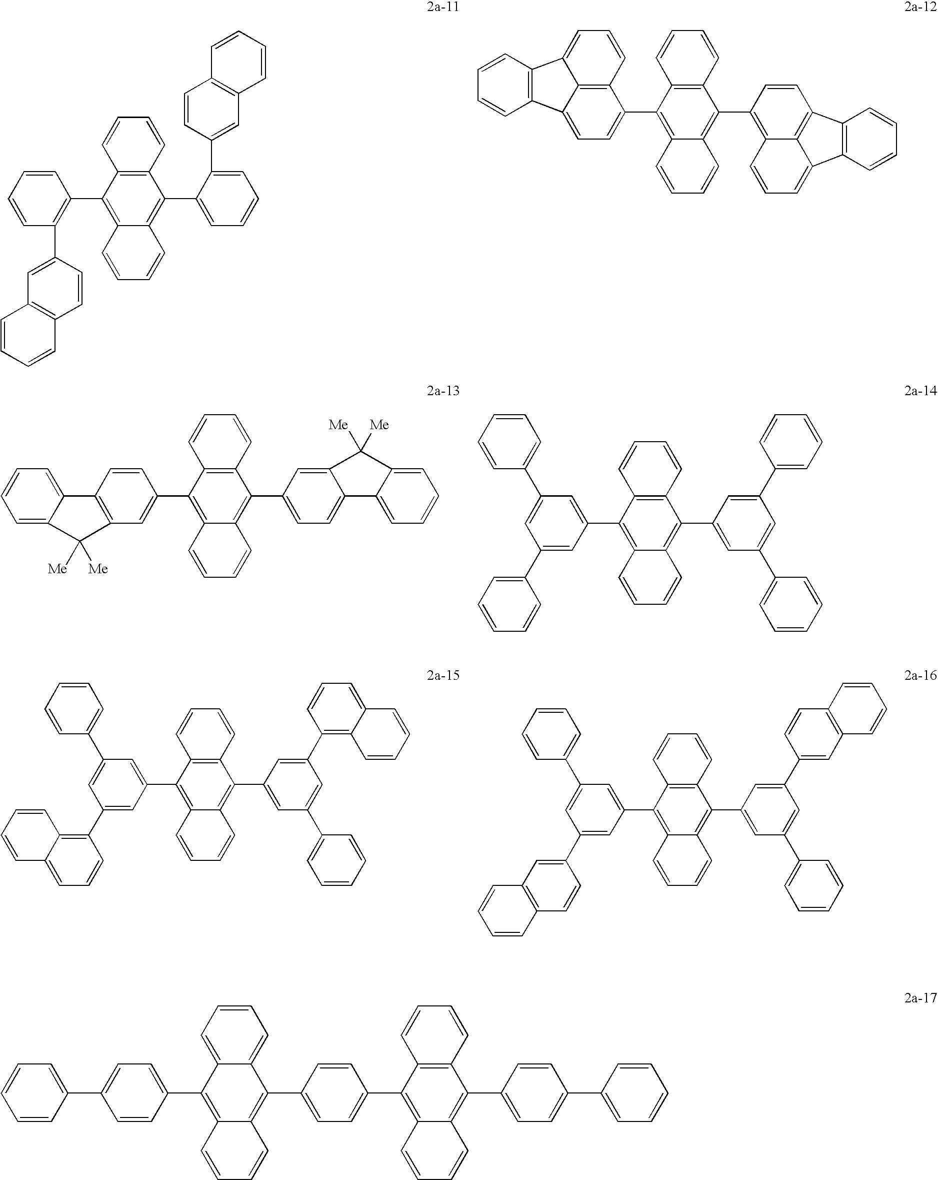

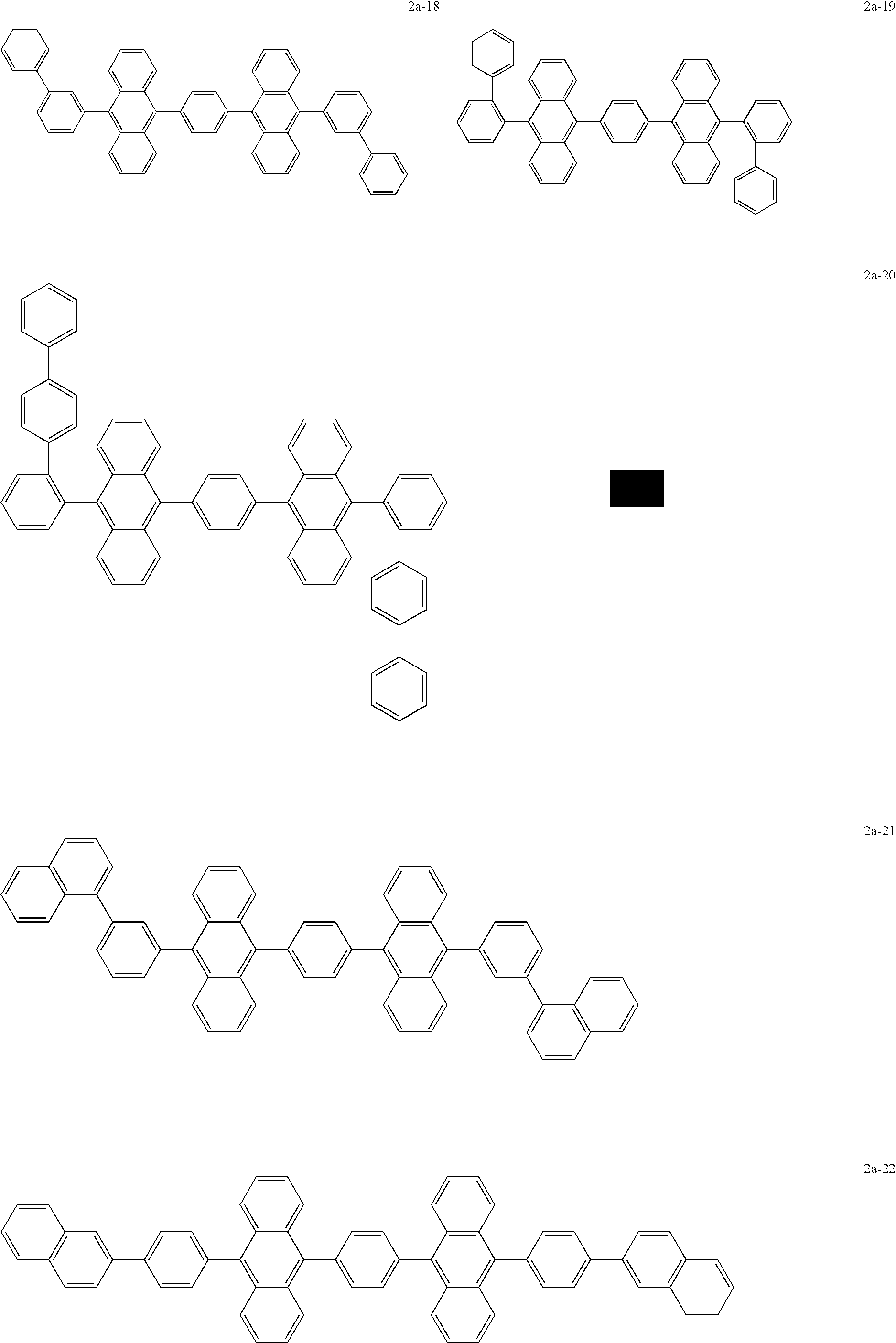

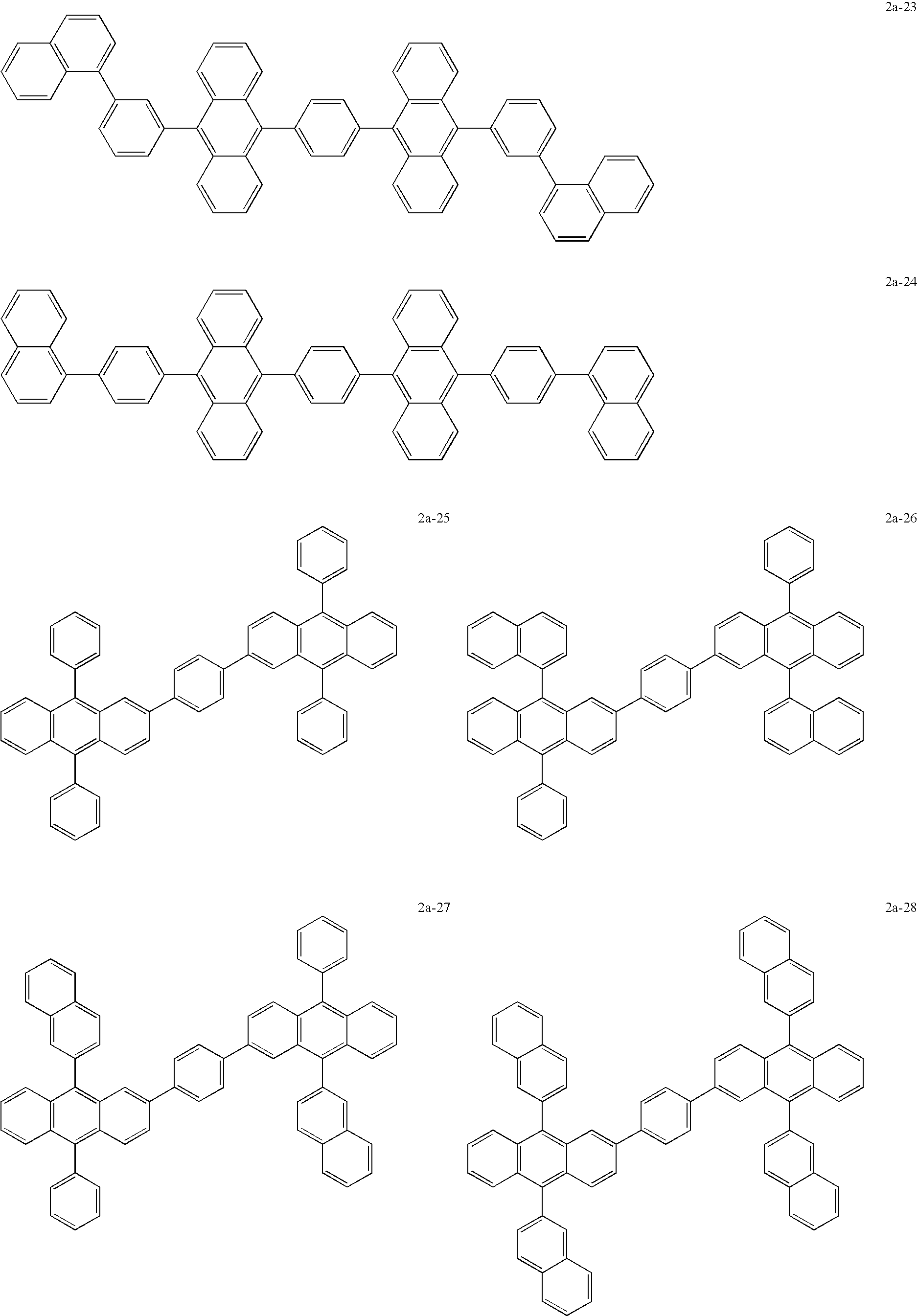

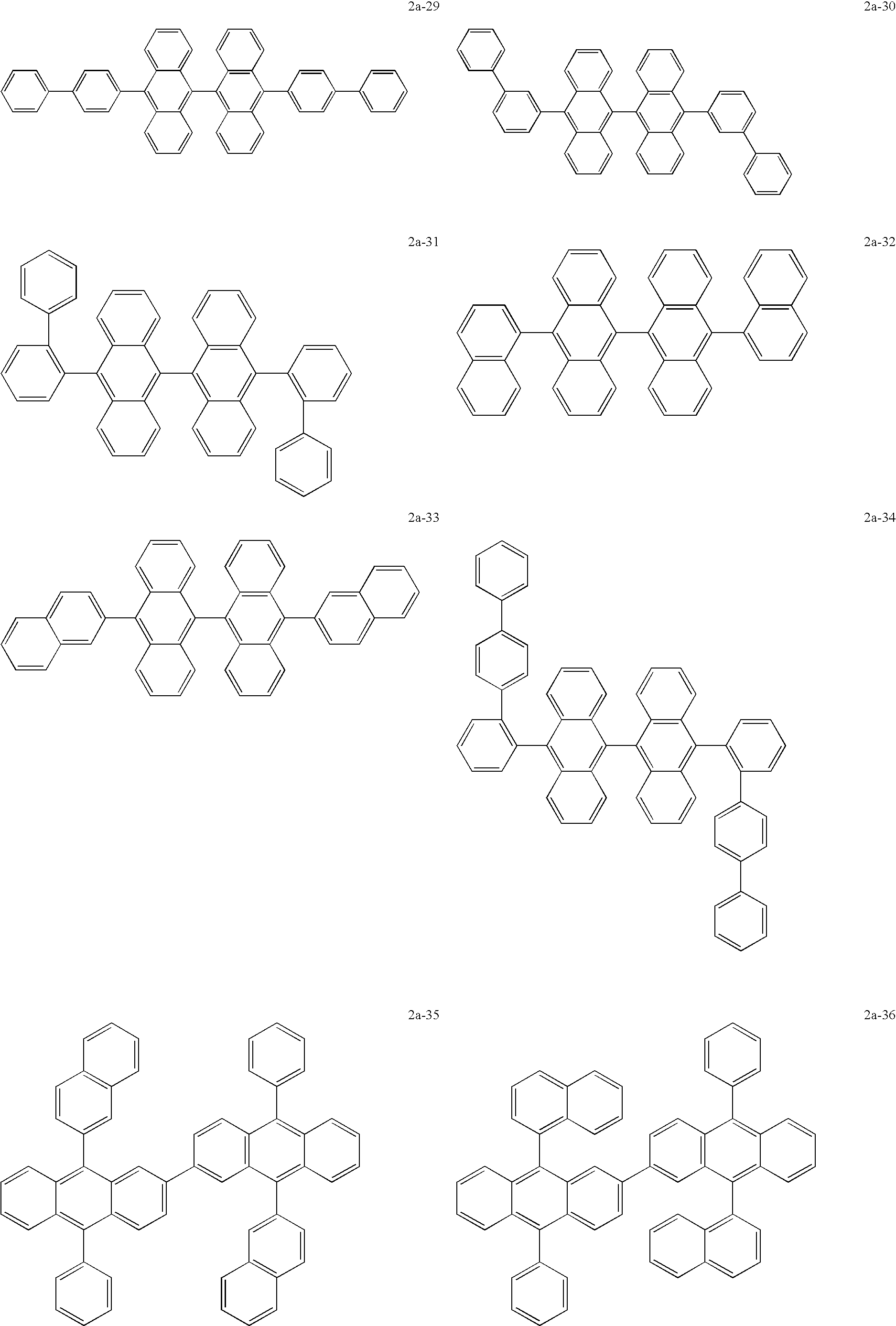

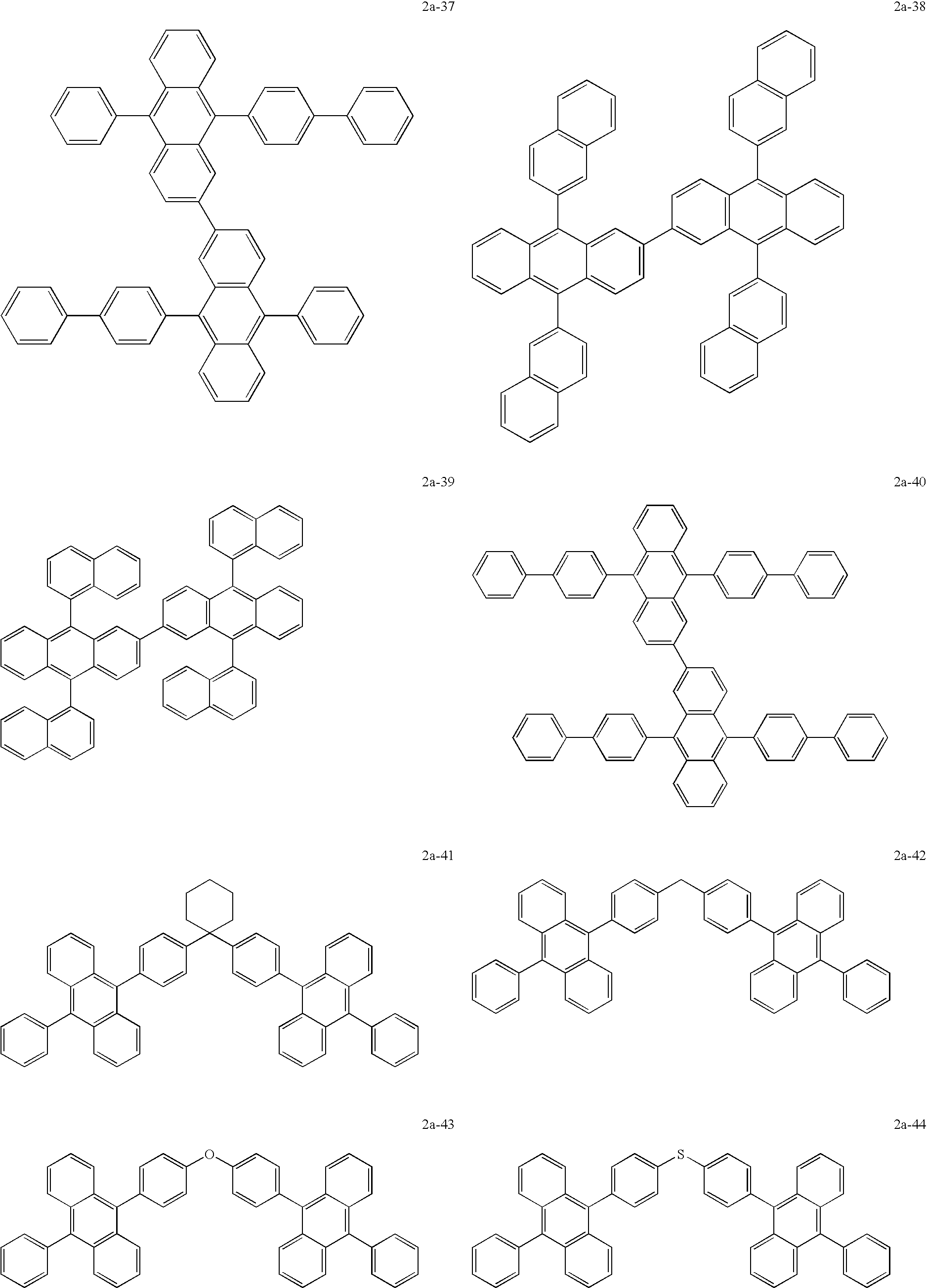

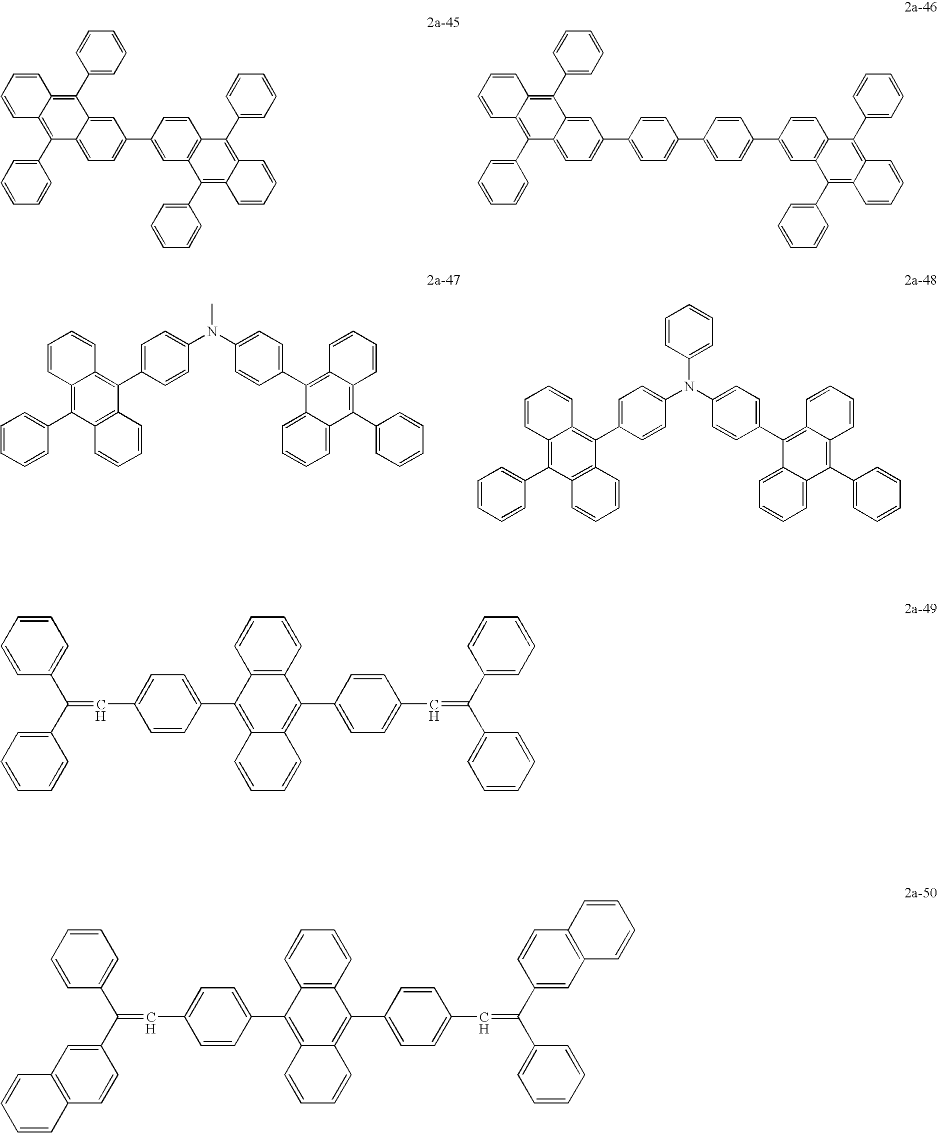

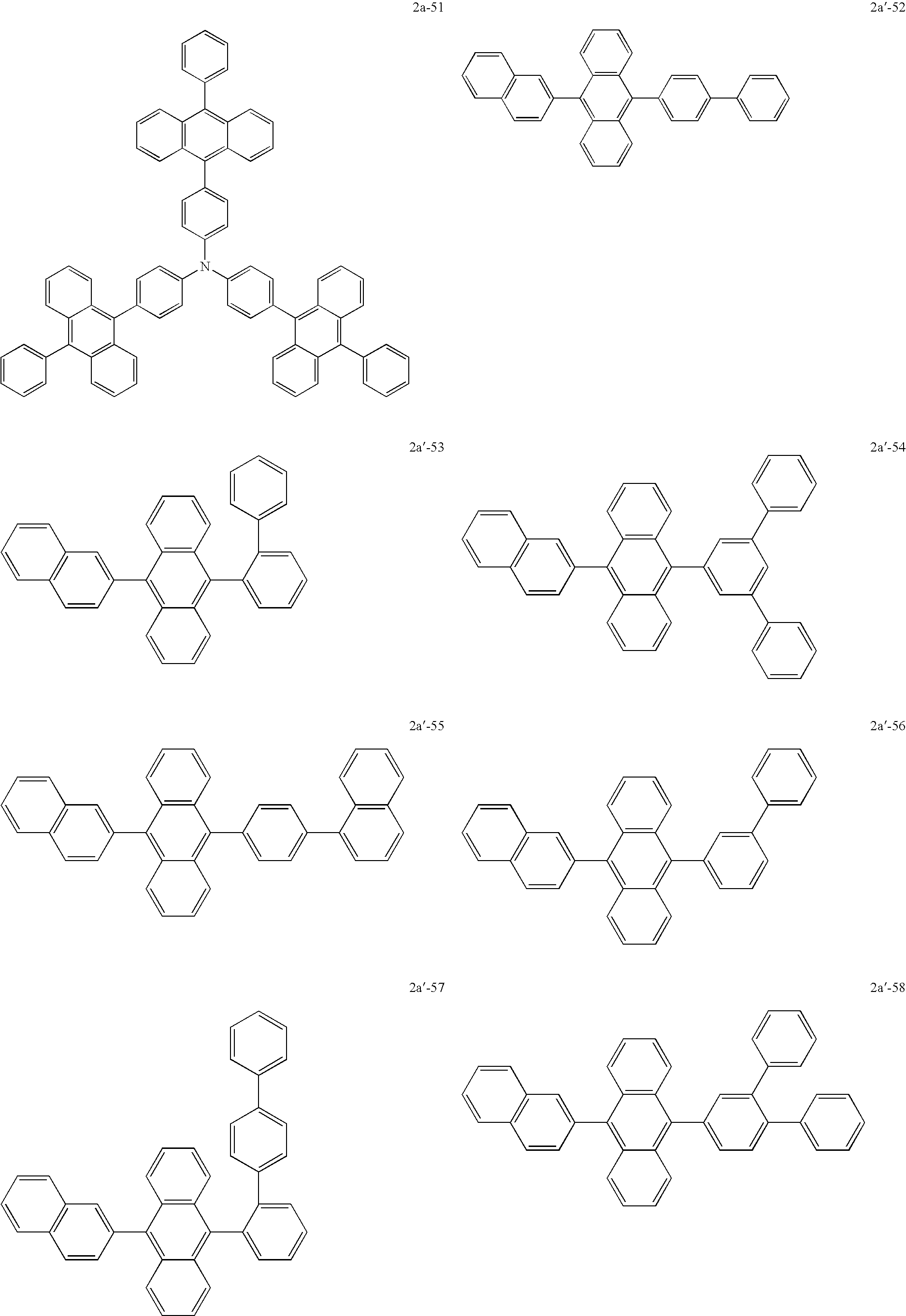

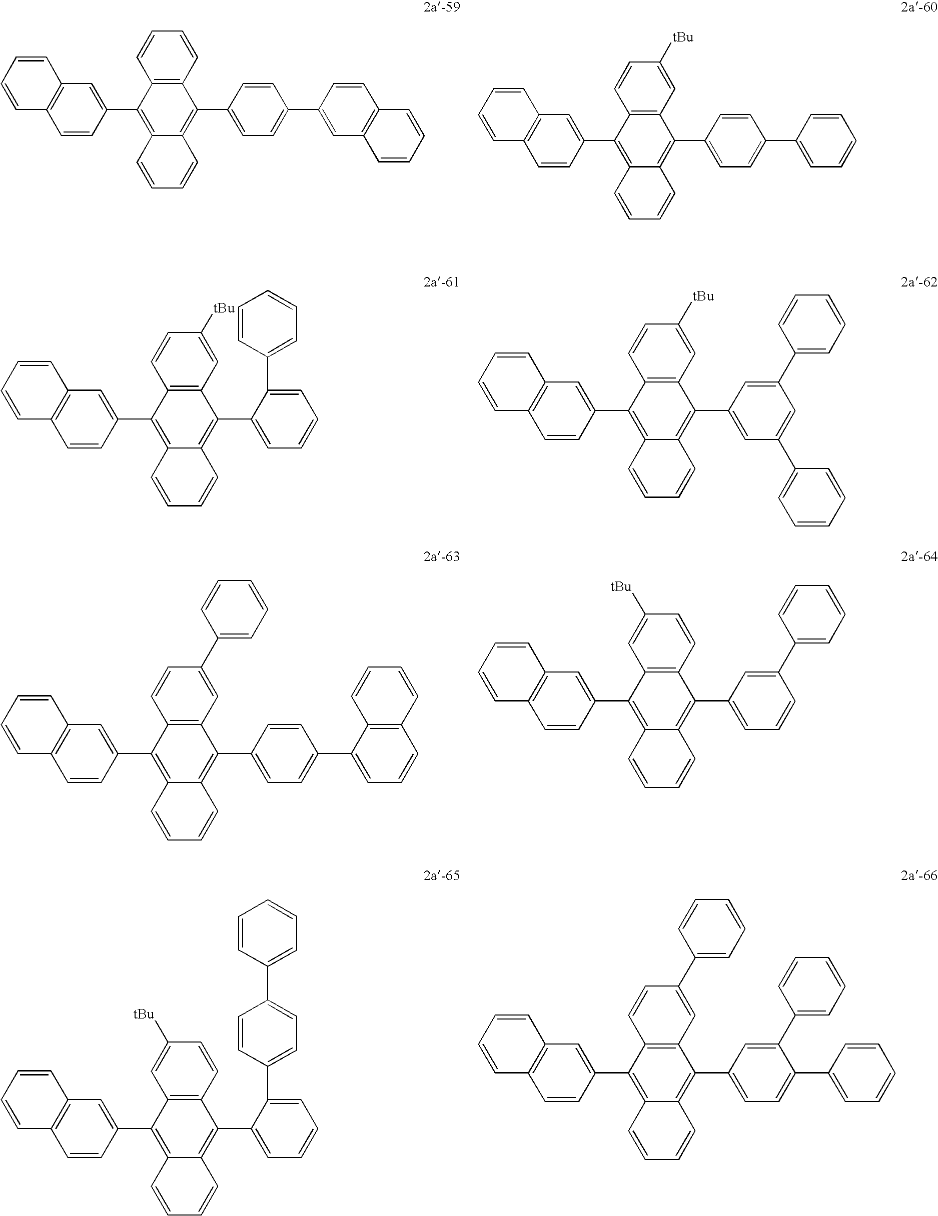

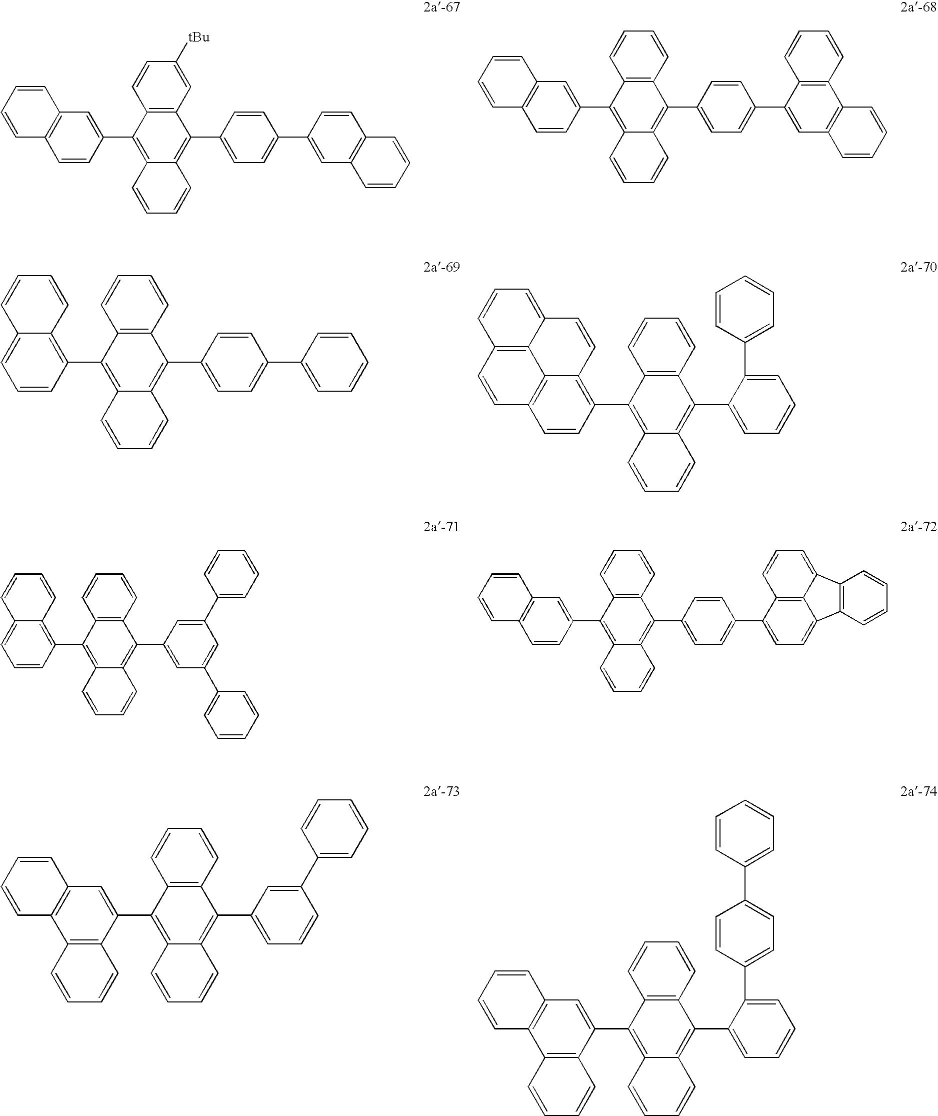

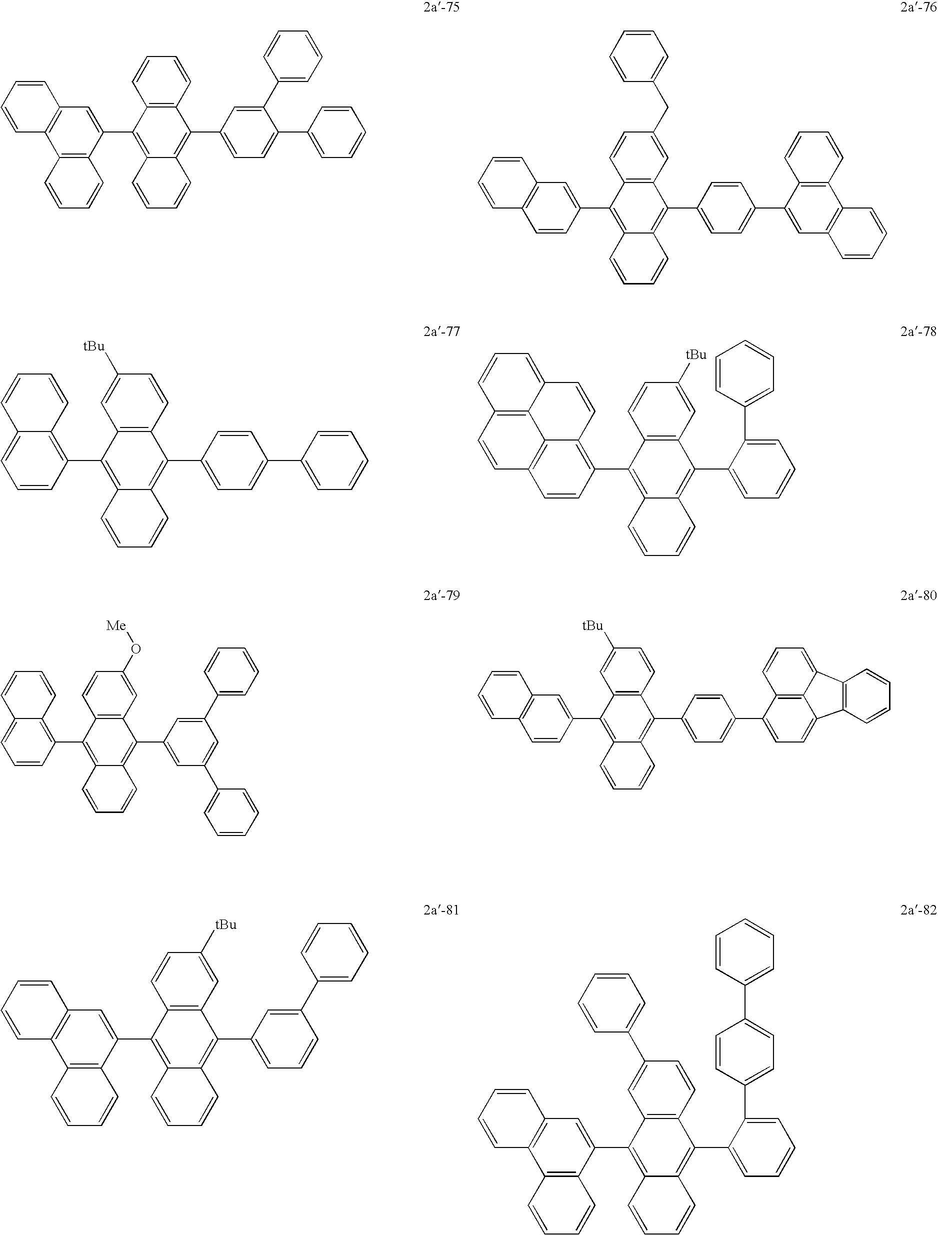

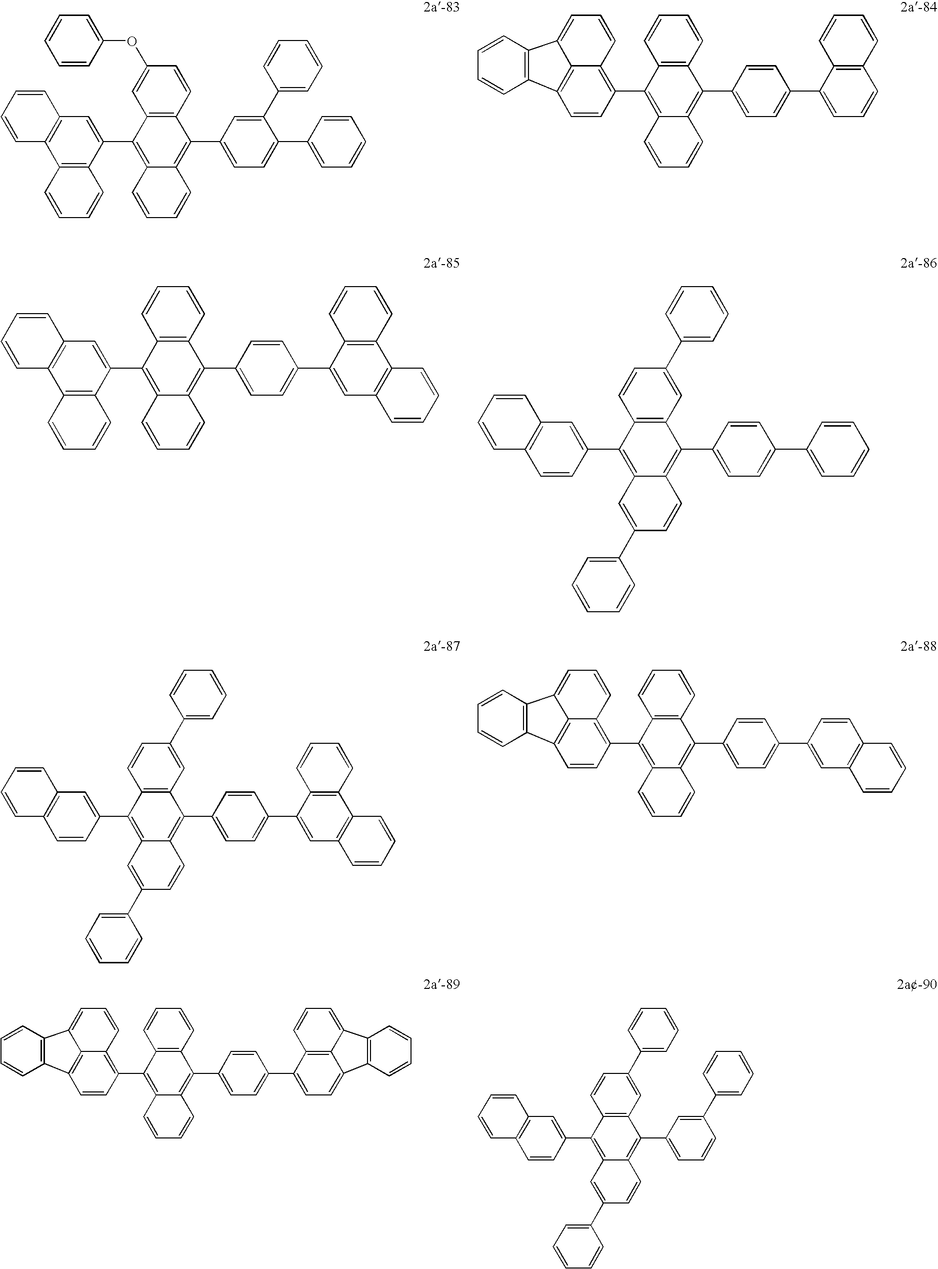

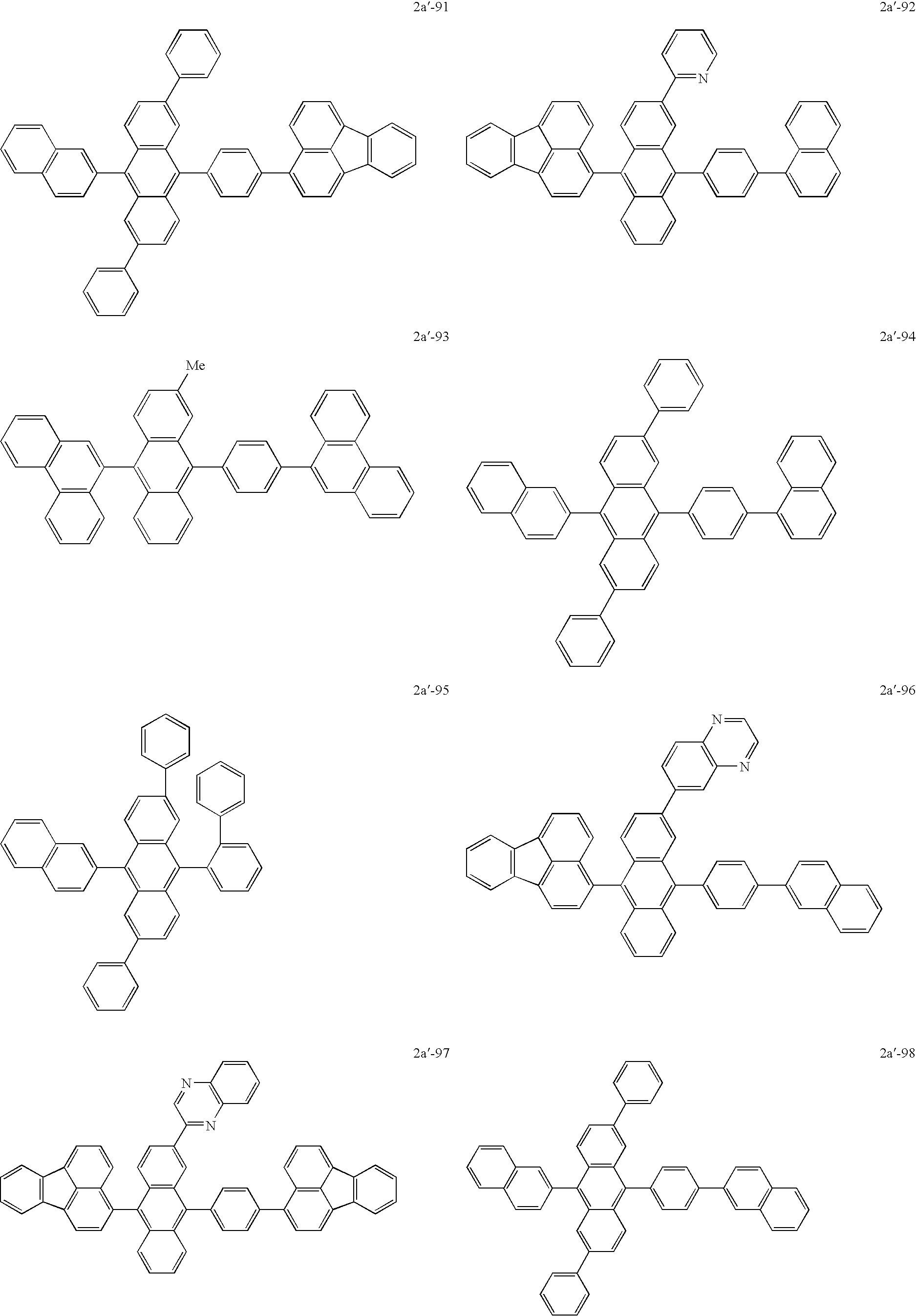

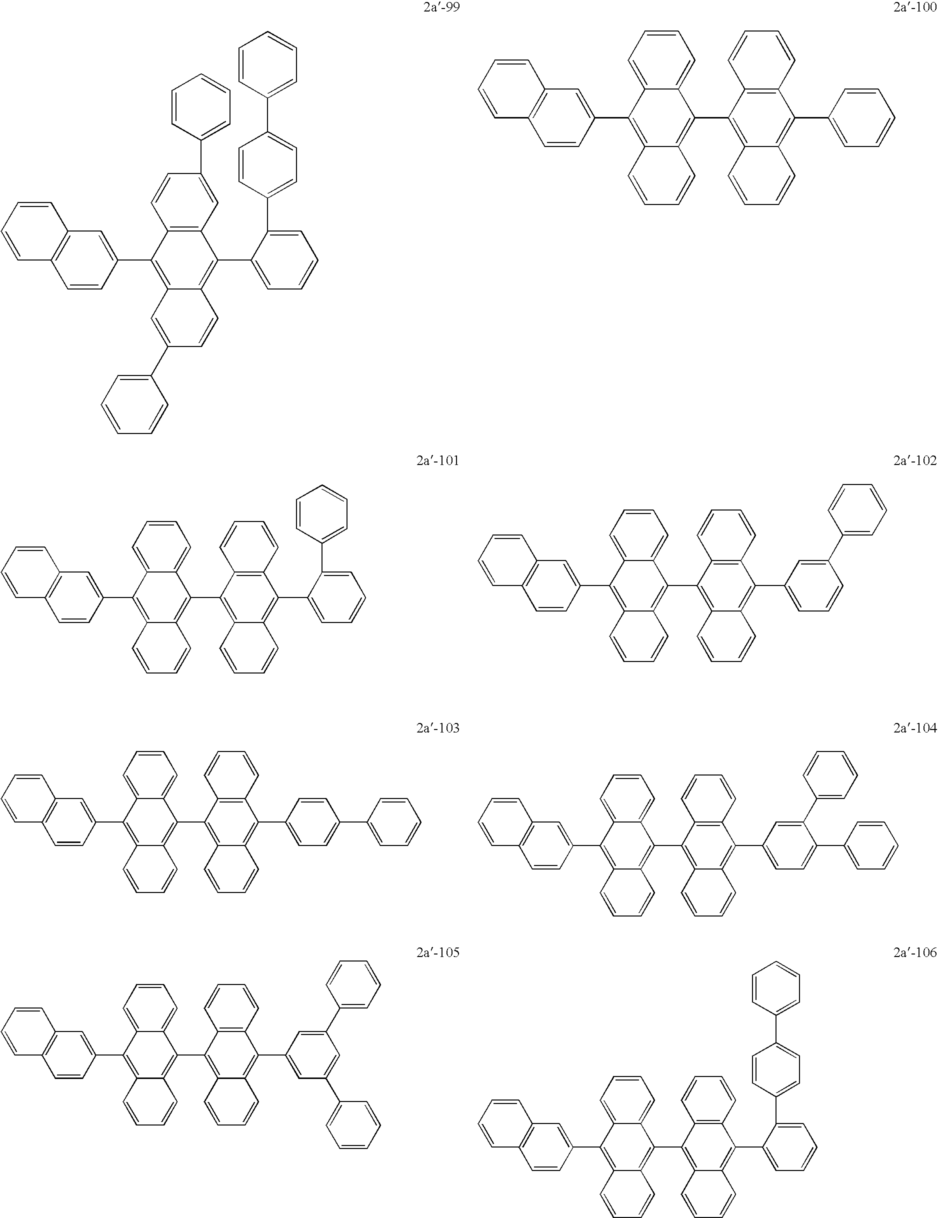

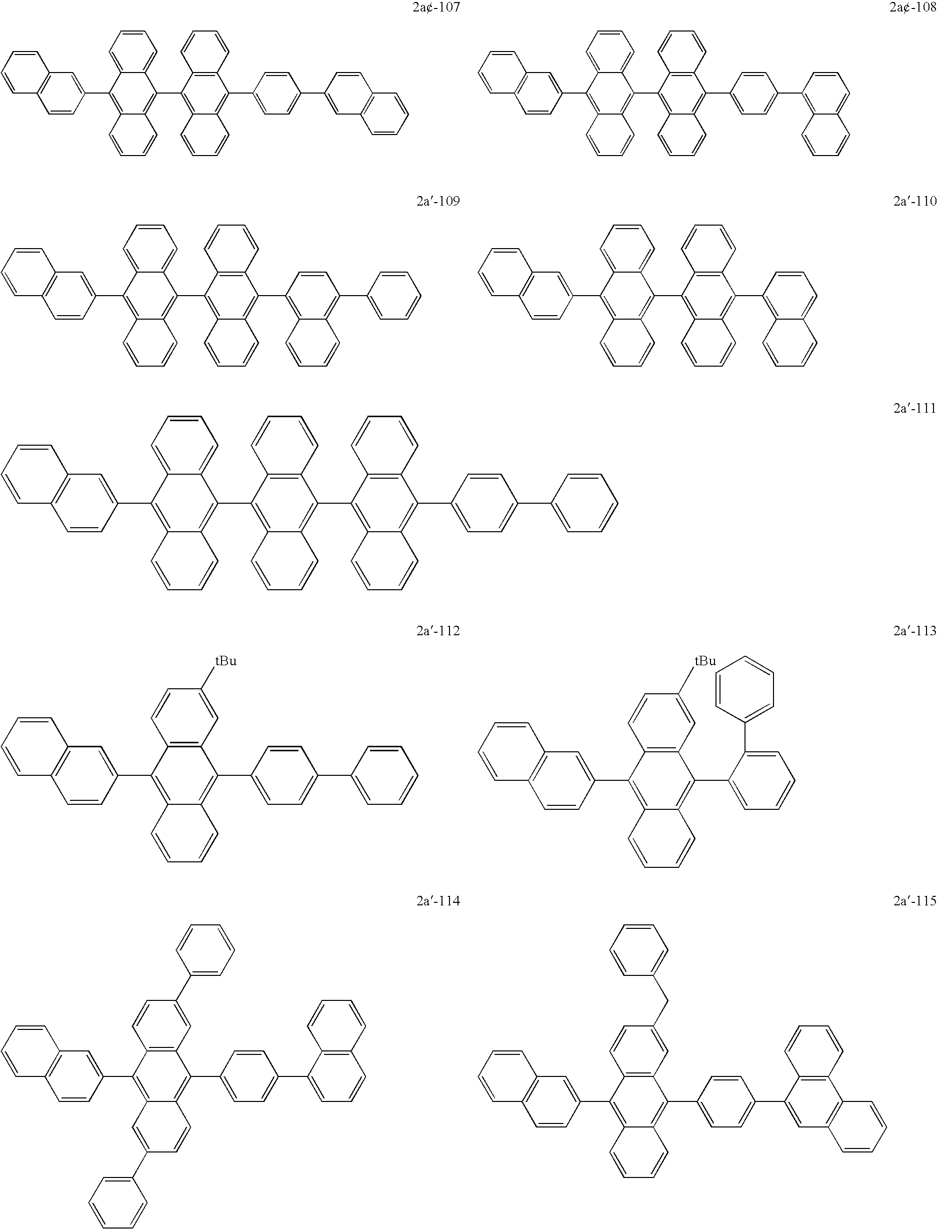

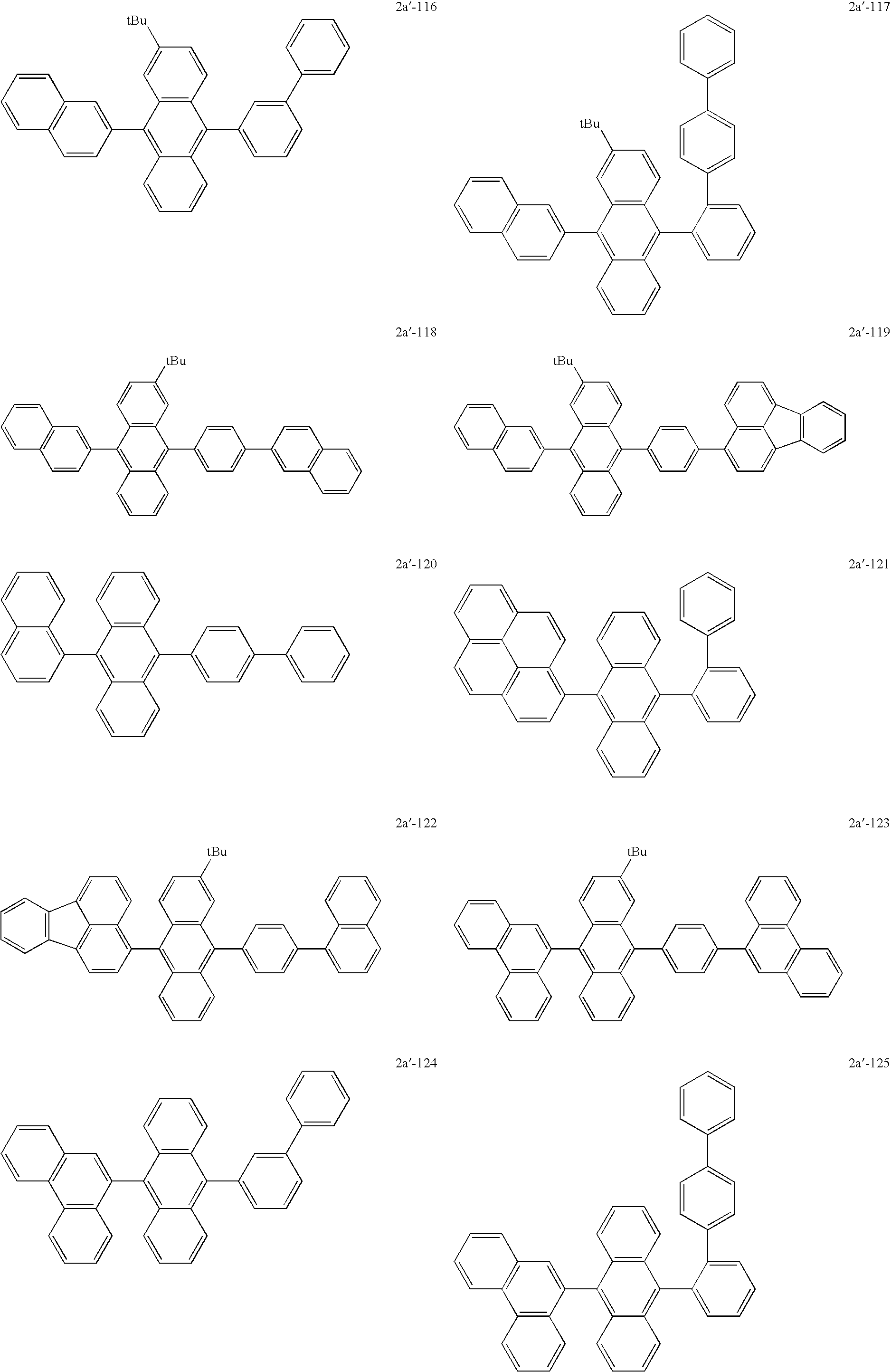

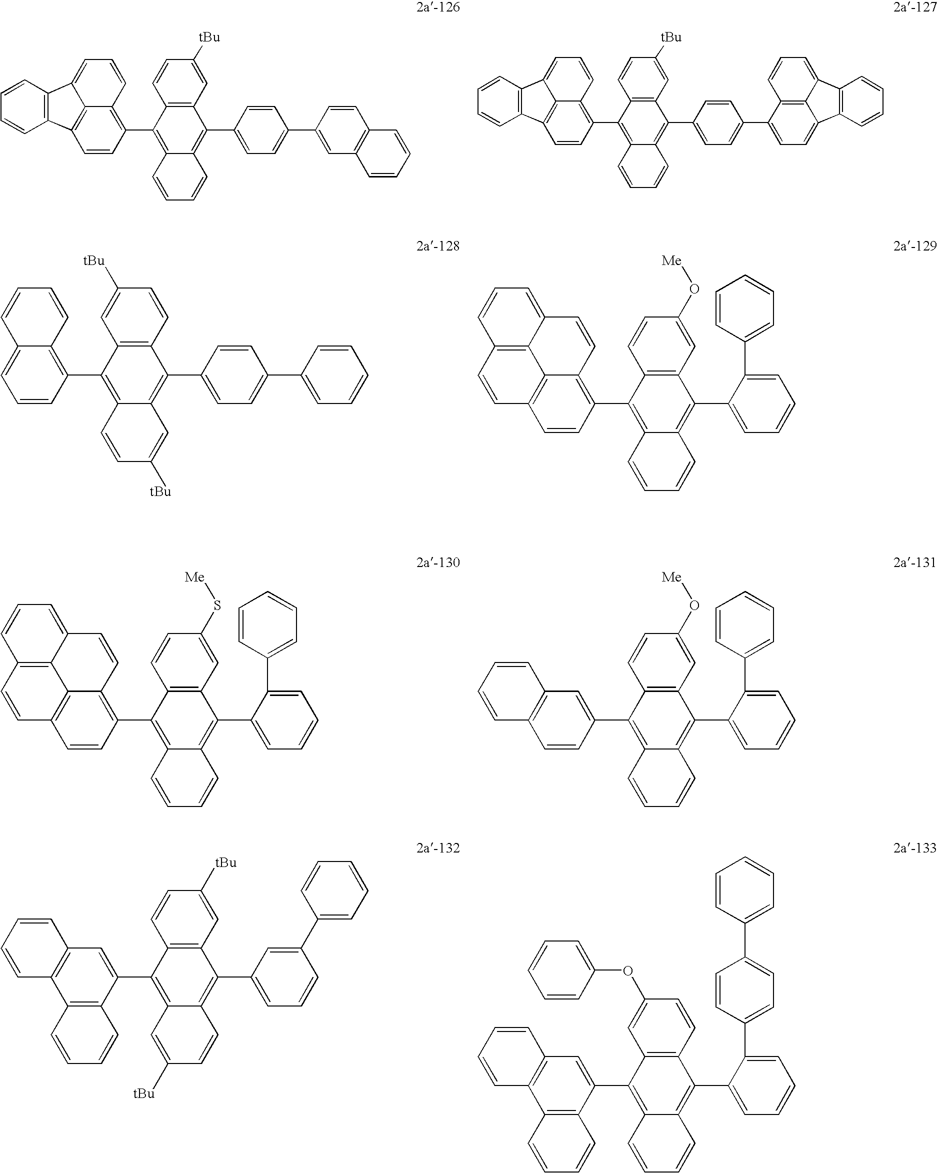

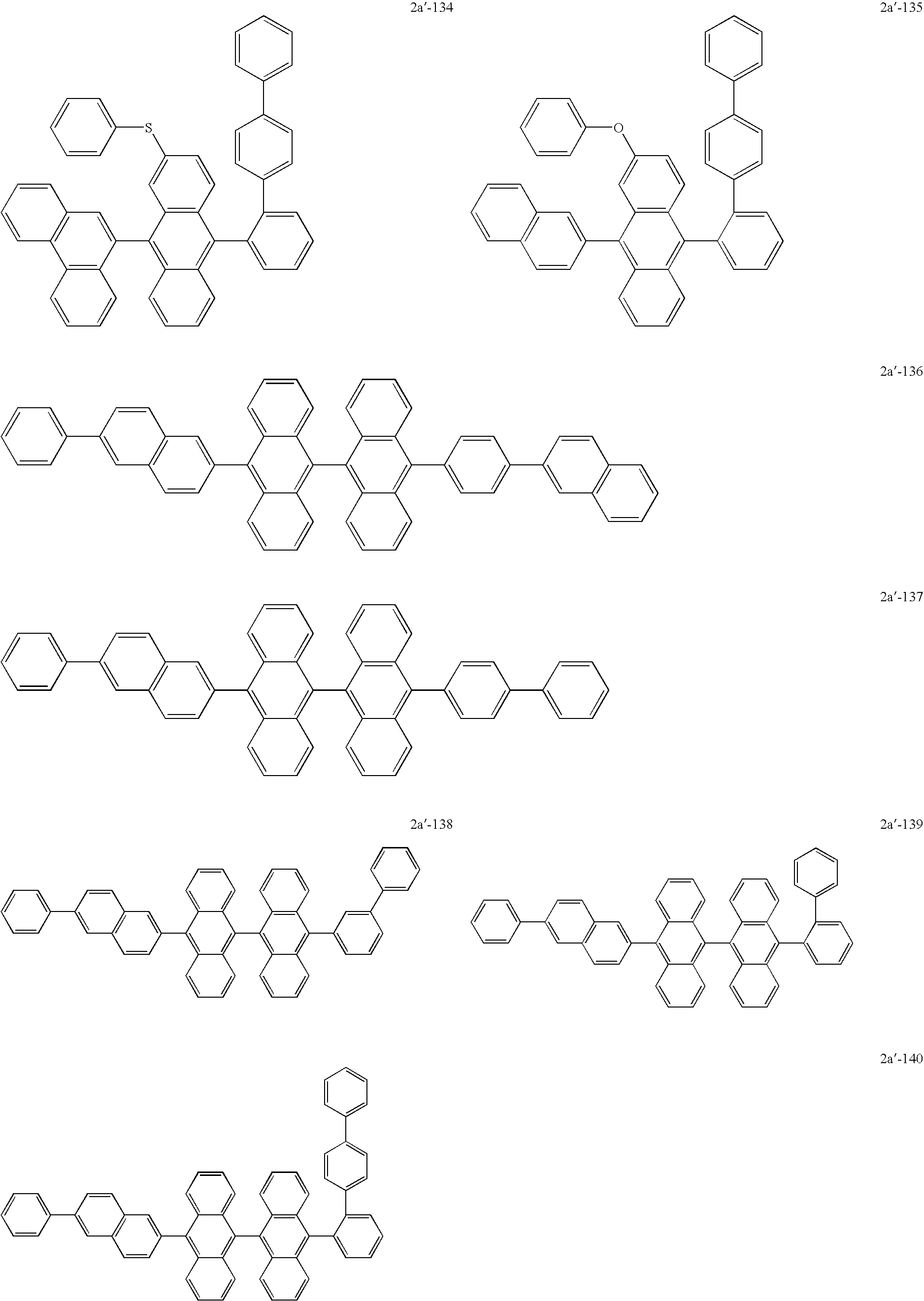

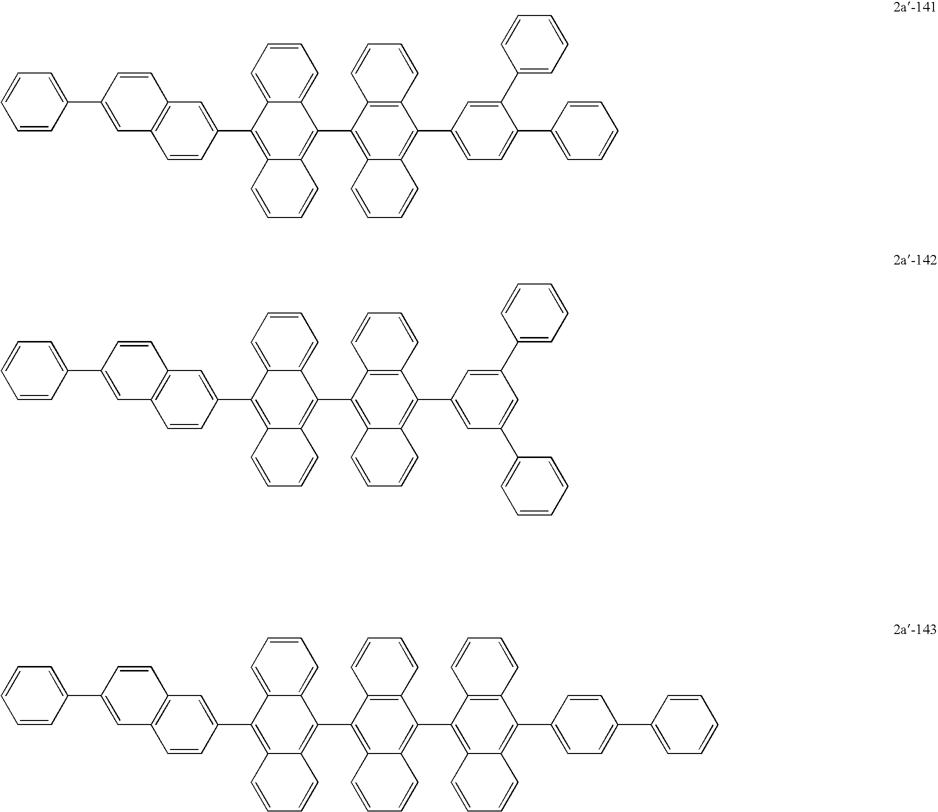

### Formula (2b)

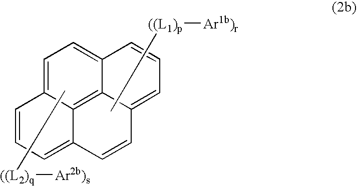

(in Formula (2b), Ar^{1b} and Ar^{2b} each are independently a substituted or non-substituted aryl group having 6 to 50 ring carbon atoms;
L₁ and L₂ each are selected independently from a substituted or non-substituted phenylene group, a substituted or non-substituted naphthalenylene group, a substituted or non-substituted fluorenylene group and a substituted or non-substituted dibenzosilolylene group;
p and q are an integer of 0 to 2, and r and s are an integer of 1 to 4;
L₁ or Ar^{1b} is bonded to any of 1- to 5-positions of pyrene, and L₂ or Ar^{2b} is bonded to any of 6- to 10-positions of pyrene).

The substituted or non-substituted aryl group having 6 to 50 ring carbon atoms represented by Ar^{1b} and Ar^{2b} in Formula (2b) includes phenyl, 1-naphthyl, 2-naphthyl, 1-anthryl, 2-anthryl, 9-anthryl, 9-(10-phenyl)anthryl, 9-(10-naphthyl-1-yl)anthryl, 9-(10-naphthyl-2-yl)anthryl, 1-phenanthryl, 2-phenanthryl, 3-phenanthryl, 4-phenanthryl, 9-phenanthryl, 1-naphthacenyl, 2-naphthacenyl, 9-naphthacenyl, 1-pyrenyl, 2-pyrenyl, 4-pyrenyl, 2-biphenylyl, 3-biphenylyl, 4-biphenylyl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-t-butylphenyl, 3-methyl-2-naphthyl, 4-methyl-1-naphthyl, 4-methyl-1-anthryl and the like. It is preferably an aromatic cyclic group having 6 to 16 ring carbon atoms, and it is particularly phenyl, 1-naphthyl, 2-naphthyl, 9-(10-phenyl)anthryl, 9-(10-naphthyl-1-yl)anthryl, 9-(10-naphthyl-2-yl)anthryl, 9-phenanthryl, 1-pyrenyl, 2-pyrenyl, 4-pyrenyl, 2-biphenylyl, 3-biphenylyl, 4-biphenylyl, o-tolyl, m-tolyl, p-tolyl and p-t-butylphenyl.

The aryl group described above may be further substituted with a substituent, and the substituent includes an alkyl group (methyl, ethyl, propyl, isopropyl, n-butyl, s-butyl, isobutyl, t-butyl, n-pentyl, n-hexyl, n-heptyl, n-octyl, hydroxymethyl, 1-hydroxyethyl, 2-hydroxyethyl, 2-hydroxyisobutyl, 1,2-dihydroxyethyl, 1,3-dihydroxyisopropyl, 2,3-dihydroxy-t-butyl, 1,2,3-trihydroxypropyl, chloromethyl, 1-chloroethyl, 2-chloroethyl, 2-chloroisobutyl, 1,2-dichloroethyl, 1,3-dichloroisopropyl, 2,3-dichloro-t-butyl, 1,2,3-trichloropropyl, bromomethyl, 1-bromoethyl, 2-bromoethyl, 2-bromoisobutyl, 1,2-dibromoethyl, 1,3-dibromoisopropyl, 2,3-dibromo-t-butyl, 1,2,3-tribromopropyl, iodomethyl, 1-iodoethyl, 2-iodoethyl, 2-iodoisobutyl, 1,2-diiodoethyl, 1,3-diiodoisopropyl, 2,3-diiodo-t-butyl, 1,2,3-triiodopropyl, aminomethyl, 1-aminoethyl, 2-aminoethyl, 2-aminoisobutyl, 1,2-diaminoethyl, 1,3-diaminoisopropyl, 2,3-diamino-t-butyl, 1,2,3-triaminopropyl, cyanomethyl, 1-cyanoethyl, 2-cyanoethyl, 2-cyanoisobutyl, 1,2-dicyanoethyl, 1,3-dicyanoisopropyl, 2,3-dicyano-t-butyl, 1,2,3-tricyanopropyl, nitromethyl, 1-nitroethyl, 2-nitroethyl, 2-nitroisobutyl, 1,2-dinitroethyl, 1,3-dinitroisopropyl, 2,3-dinitro-t-butyl, 1,2,3-trinitropropyl, cyclopropyl, cyclobutyl, cyclopentyl, cyclohexyl, 4-methylcyclohexyl, 1-adamantyl, 2-adamantyl, 1-norbornyl, 2-norbornyl and the like), an alkoxy group having 1 to 6 carbon atoms (ethoxy, methoxy, i-propoxy, n-propoxy, s-butoxy, t-butoxy, pentoxy, hexyloxy, cyclopentoxy, cyclohexyloxy and the like), an aryl group having 5 to 40 ring carbon atoms, an amino group substituted with an aryl group having 5 to 40 ring carbon atoms, an ester group having an aryl group having 5 to 40 ring carbon atoms, an ester group having an alkyl group having 1 to 6 carbon atoms, a cyano group, a nitro group, a halogen atom and the like.

L₁ and L₂ in Formula (2b) are selected preferably from a substituted or non-substituted phenylene group and a substituted or non-substituted fluorenylene group.

The substituents therefor include the same groups as given in the aromatic group described above.

The term p in Formula (2b) is preferably an integer of 0 to 1. The term r in Formula (2b) is preferably an integer of 1 to 2. The term q in Formula (2b) is preferably an integer of 0 to 1. The term s in Formula (2b) is preferably an integer of 0 to 2.

The specific examples of the pyrene derivative represented by Formula (2b) used for the organic EL device of the present invention include asymmetric pyrene derivatives shown in the paragraphs of [0020] to [0023] of International Publication NO. 2005/115950. In addition thereto, symmetric pyrene derivatives can also be used as a material for the organic EL device of the present invention. The representative specific examples thereof are shown below.

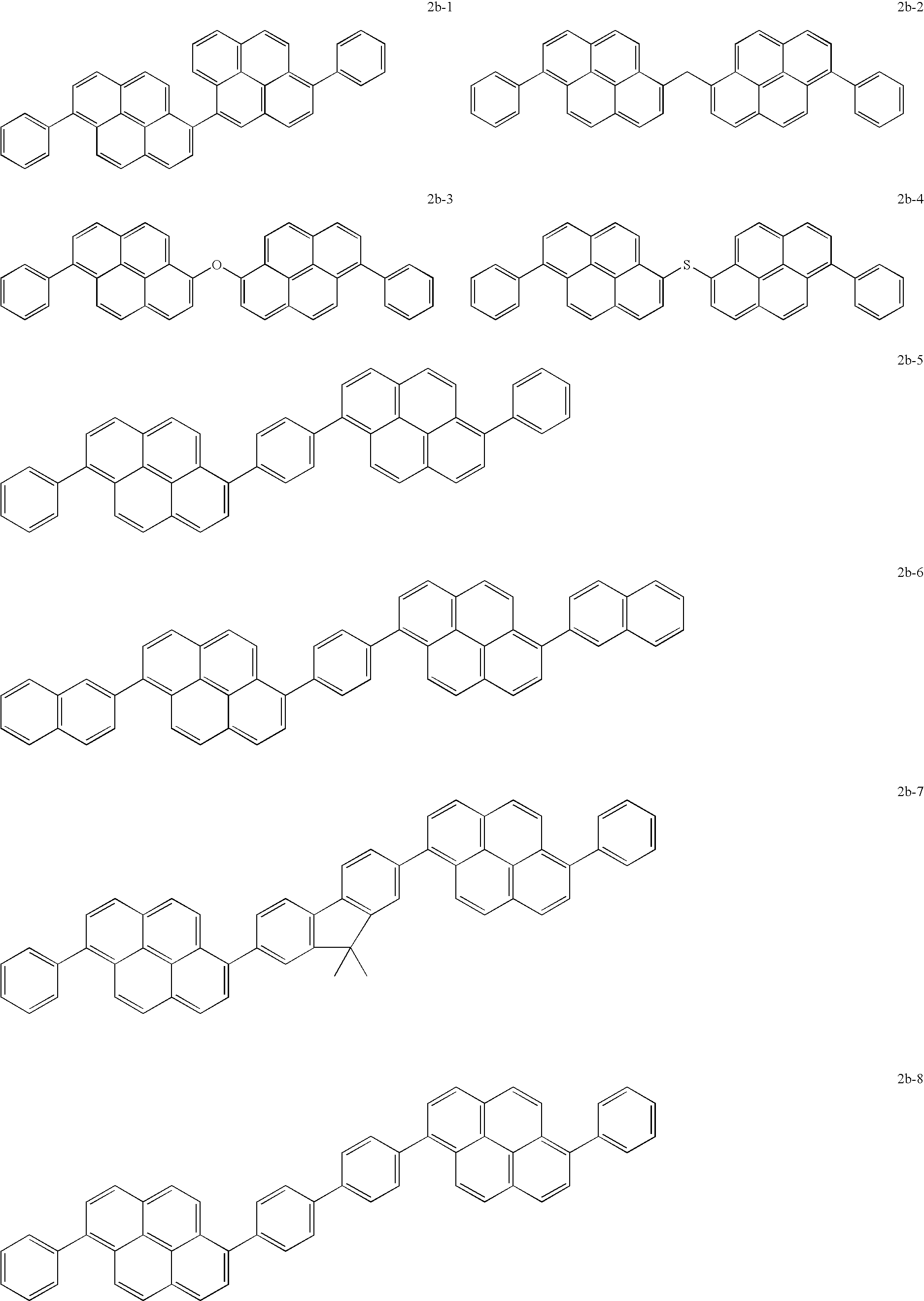

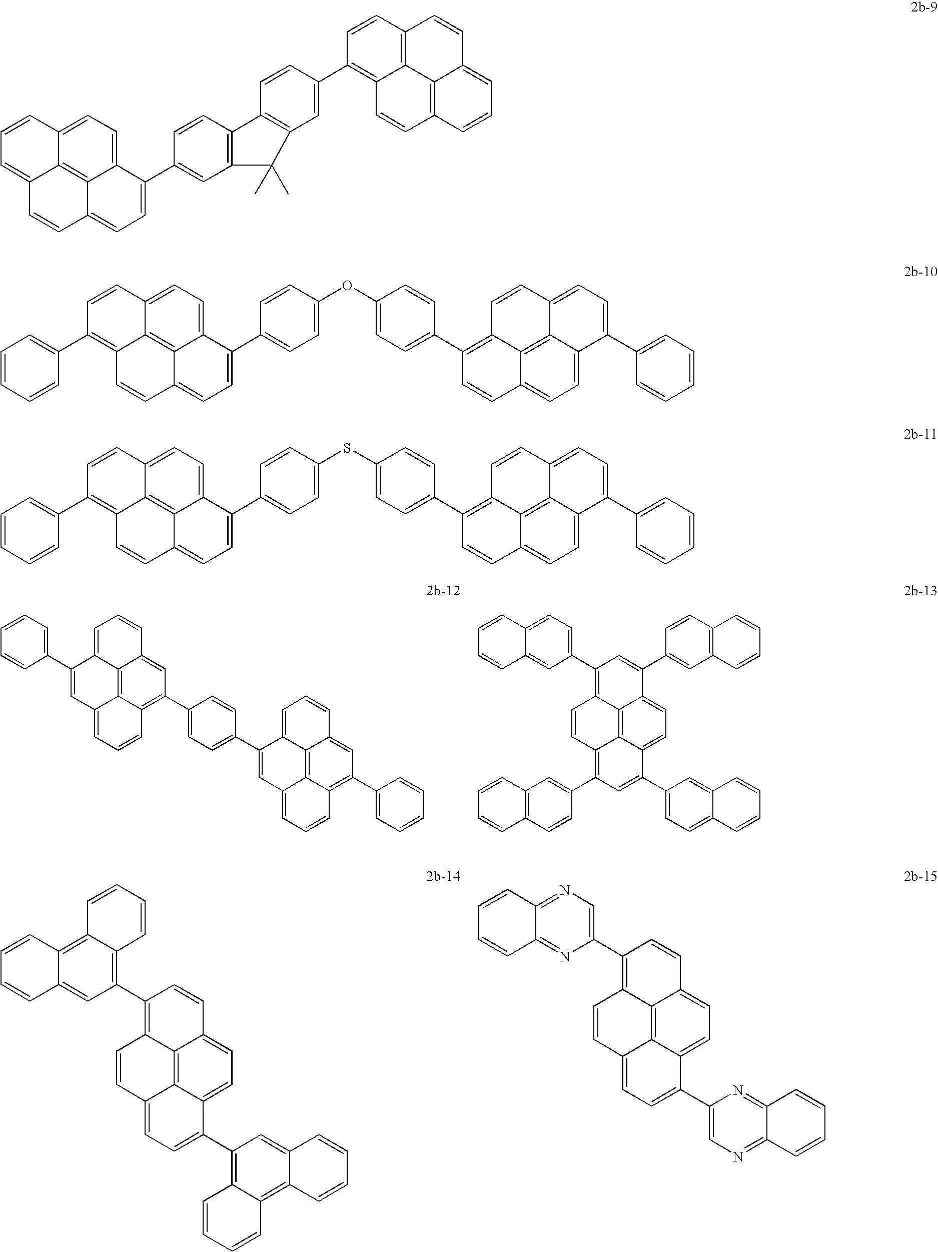

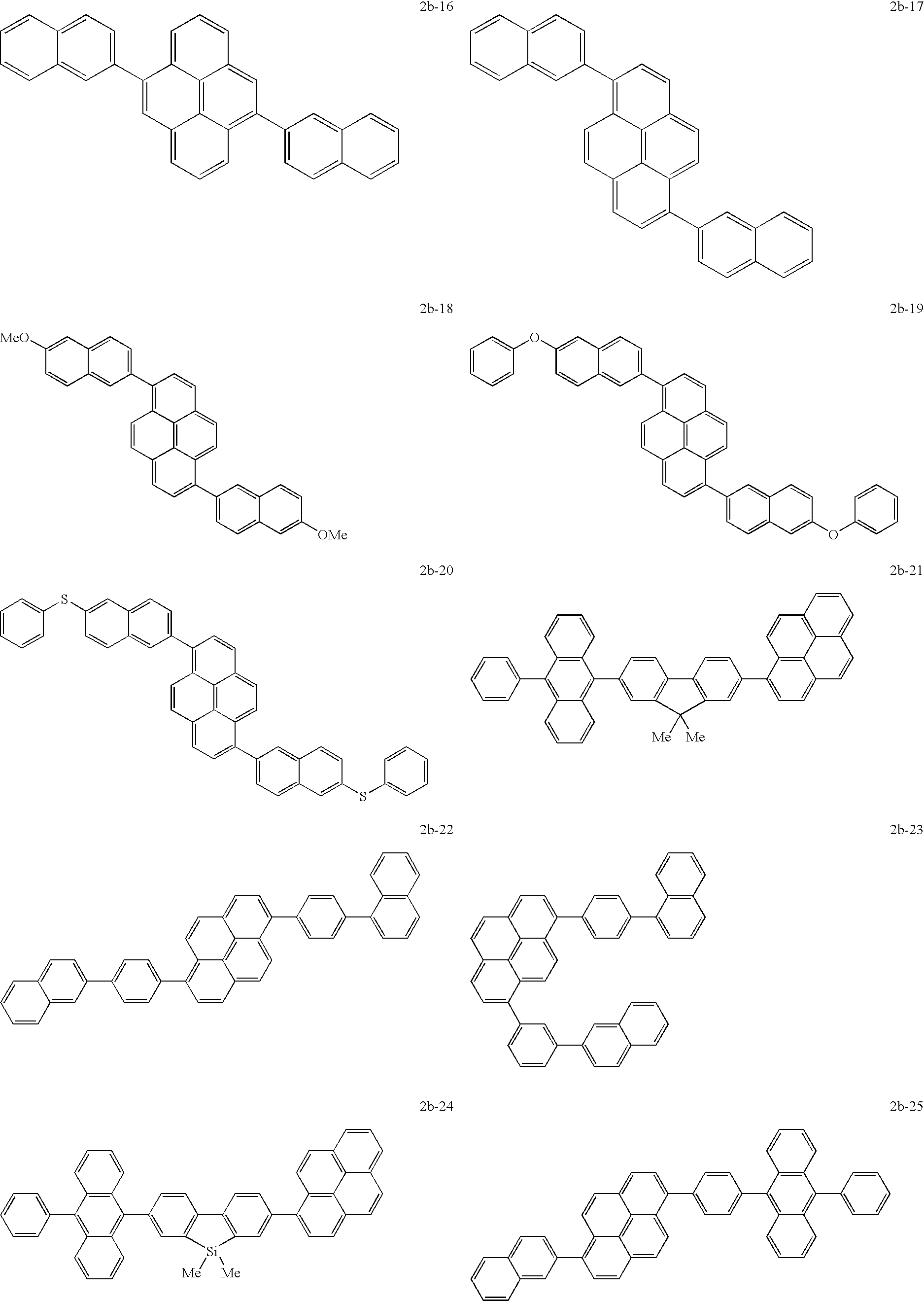

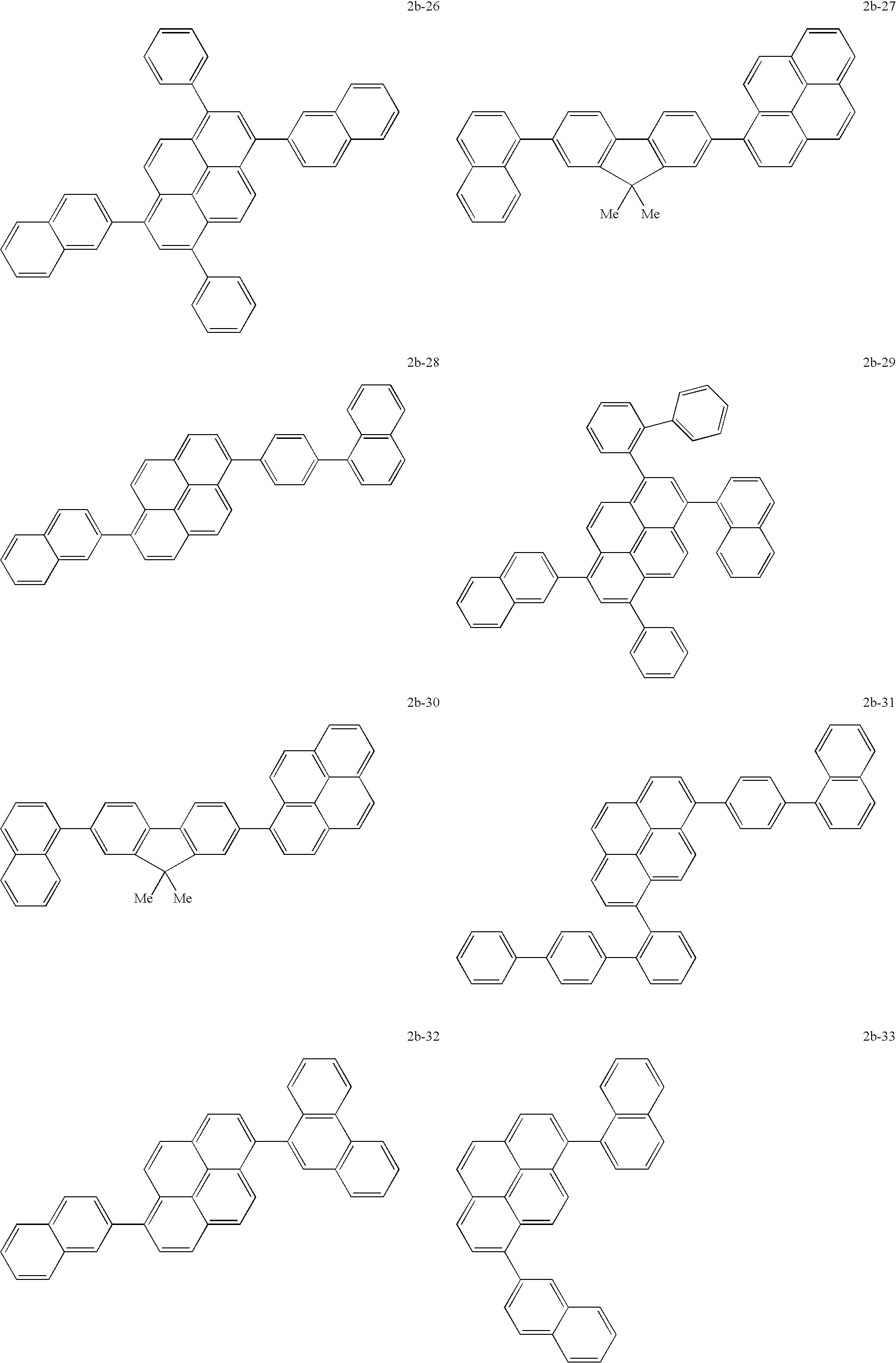

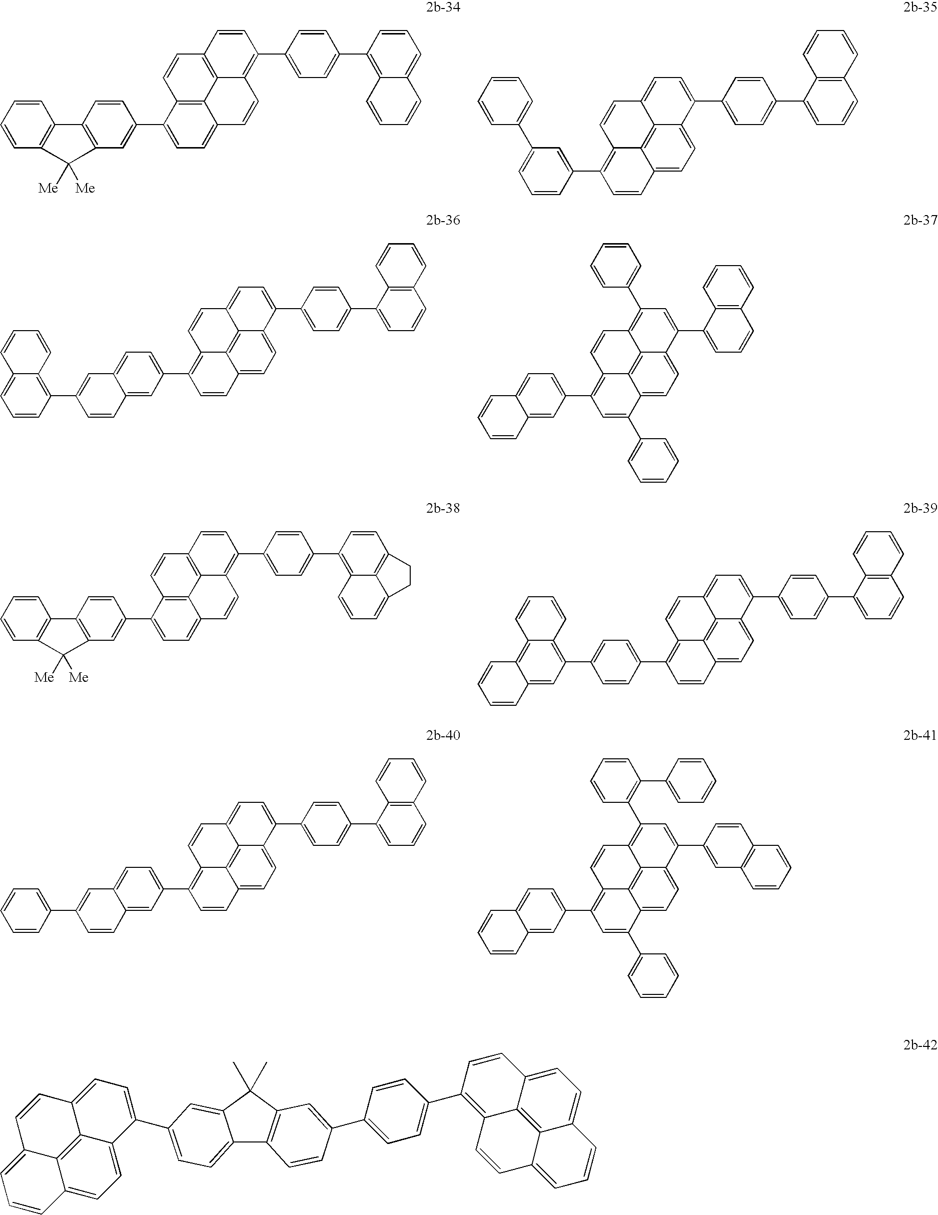

### Formula (2c)

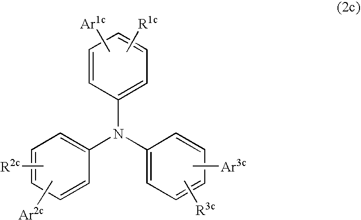

(in Formula (2c), Ar^{1c}, Ar^{2c} and Ar^{3c} each are selected independently from a group having an anthracene structure, a group having a phenanthrene structure, a group having a pyrene structure, a group having a fluorene structure and a group having a perylene structure; and
R^{1c}, R^{2c} and R^{3c} each represent independently a hydrogen atom or a substituent).

Ar^{1c}, Ar^{2c} and Ar^{3c} in Formula (2c) are selected preferably from a substituted or non-substituted anthrylphenyl group, an anthryl group, a phenanthrenyl group, a perylenyl group and a pyrenyl group, and they are selected more preferably from alkyl-substituted or non-substituted anthrylphenyl, phenanthryl, fluorene, perylene and pyrenyl. They are selected particularly preferably from a pyrenyl group and a phenanthryl group.

The substituents R^{1c}, R^{2c} and R^{3c} in Formula (2c) include an alkyl group (it has preferably 1 to 30 carbon atoms, more preferably 1 to 20 carbon atoms and particularly preferably 1 to 10 carbon atoms and includes, for example, methyl, ethyl, isopropyl, t-butyl, n-octyl, n-decyl, n-hexadecyl, cyclopropyl, cyclopentyl, cyclohexyl and the like), an alkenyl group (it has preferably 2 to 30 carbon atoms, more preferably 2 to 20 carbon atoms and particularly preferably 2 to 10 carbon atoms and includes, for example, vinyl, allyl, 2-butenyl, 3-pentenyl and the like), an alkynyl group (it has preferably 2 to 30 carbon atoms, more preferably 2 to 20 carbon atoms and particularly preferably 2 to 10 carbon atoms and includes, for example, propargyl, 3-pentynyl and the like), an aryl group (it has preferably 6 to 30 carbon atoms, more preferably 6 to 20 carbon atoms and particularly preferably 6 to 12 carbon atoms and includes, for example, phenyl, p-methylphenyl, naphthyl, anthranyl and the like), an amino group (it has preferably 0 to 30 carbon atoms, more preferably 0 to 20 carbon atoms and particularly preferably 0 to 10 carbon atoms and includes, for example, amino, methylamino, dimethylamino, diethylamino, dibenzylamino, diphenylamino, ditolylamino and the like), an alkoxy group (it has preferably 1 to 30 carbon atoms, more preferably 1 to 20 carbon atoms and particularly preferably 1 to 10 carbon atoms and includes, for example, methoxy, ethoxy, butoxy, 2-ethylhexyloxy and the like), an aryloxy group (it has preferably 6 to 30 carbon atoms, more preferably 6 to 20 carbon atoms and particularly preferably 6 to 12 carbon atoms and includes, for example, phenyloxy, 1-naphthyloxy, 2-naphthyloxy and the like), a heteroaryloxy group (it has preferably 1 to 30 carbon atoms, more preferably 1 to 20 carbon atoms and particularly preferably 1 to 12 carbon atoms and includes, for example, pyridyloxy, pyrazyloxy, pyrimidyloxy, quinolyloxy and the like), an acyl group (it has preferably 1 to 30 carbon atoms, more preferably 1 to 20 carbon atoms and particularly preferably 1 to 12 carbon atoms and includes, for example, acetyl, benzoyl, formyl, pivaloyl and the like), an alkoxycarbonyl group (it has preferably 2 to 30 carbon atoms, more preferably 2 to 20 carbon atoms and particularly preferably 2 to 12 carbon atoms and includes, for example, methoxycarbonyl, ethoxycarbonyl and the like), an aryloxycarbonyl group (it has preferably 7 to 30 carbon atoms, more preferably 7 to 20 carbon atoms and particularly preferably 7 to 12 carbon atoms and includes, for example, phenyloxycarbonyl and the like), an acyloxy group (it has preferably 2 to 30 carbon atoms, more preferably 2 to 20 carbon atoms and particularly preferably 2 to 10 carbon atoms and includes, for example, acetoxy, benzoyloxy and the like), an acylamino group (it has preferably 2 to 30 carbon atoms, more preferably 2 to 20 carbon atoms and particularly preferably 2 to 10 carbon atoms and includes, for example, acetylamino, benzoylamino and the like), an alkoxycarbonylamino group (it has preferably 2 to 30 carbon atoms, more preferably 2 to 20 carbon atoms and particularly preferably 2 to 12 carbon atoms and includes, for example, methoxycarbonylamino and the like), an aryloxycarbonylamino group (it has preferably 7 to 30 carbon atoms, more preferably 7 to 20 carbon atoms and particularly preferably 7 to 12 carbon atoms and includes, for example, phenyloxycarbonylamino and the like), a sulfonylamino group (it has preferably 1 to 30 carbon atoms, more preferably 1 to 20 carbon atoms and particularly preferably 1 to 12 carbon atoms and includes, for example, methanesulfonylamino, benzenesulfonylamino and the like), a sulfamoyl group (it has preferably 0 to 30 carbon atoms, more preferably 0 to 20 carbon atoms and particularly preferably 0 to 12 carbon atoms and includes, for example, sulfamoyl, methylsulfamoyl, dimethylsulfamoyl, phenylsulfamoyl and the like), a carbamoyl group (it has preferably 1 to 30 carbon atoms, more preferably 1 to 20 carbon atoms and particularly preferably 1 to 12 carbon atoms and includes, for example, carbamoyl, methylcarbamoyl, diethylcarbamoyl, phenylcarbamoyl and the like), an alkylthio group (it has preferably 1 to 30 carbon atoms, more preferably 1 to 20 carbon atoms and particularly preferably 1 to 12 carbon atoms and includes, for example, methylthio, ethylthio and the like), an arylthio group (it has preferably 6 to 30 carbon atoms, more preferably 6 to 20 carbon atoms and particularly preferably, 6 to 12 carbon atoms and includes, for example, phenylthio and the like), a heteroarylthio group (it has preferably 1 to 30 carbon atoms, more preferably 1 to 20 carbon atoms and particularly preferably 1 to 12 carbon atoms and includes, for example, pyridylthio, 2-benzimidazolylthio, 2-benzoxazolylthio, 2-benzothiazolylthio and the like), a sulfonyl group (it has preferably 1 to 30 carbon atoms, more preferably 1 to 20 carbon atoms and particularly preferably 1 to 12 carbon atoms and includes, for example, mesyl, tosyl and the like), a sulfinyl group (it has preferably 1 to 30 carbon atoms, more preferably 1 to 20 carbon atoms and particularly preferably 1 to 12 carbon atoms and includes, for example, methanesulfinyl, benznesulfinyl and the like), a ureido group (it has preferably 1 to 30 carbon atoms, more preferably 1 to 20 carbon atoms and particularly preferably 1 to 12 carbon atoms and includes, for example, ureido, methylureido, phenylureido and the like), a phosphoric amide group (it has preferably 1 to 30 carbon atoms, more preferably 1 to 20 carbon atoms and particularly preferably 1 to 12 carbon atoms and includes, for example, diethylphosphoric amide, phenylphosphoric amide and the like), a hydroxy group, a mercapto group, a halogen atom (including, for example, a fluorine atom, a chlorine atom, a bromine atom and an iodine atom), a cyano group, a sulfo group, a carboxy group, a nitro group, a hydroxamic acid group, a sulfino group, a hydrazino group, an imino group, a heterocyclic group (it has preferably 1 to 30 carbon atoms, more preferably 1 to 12 carbon atoms, and a hetero atom includes, for example, a nitrogen atom, an oxygen atom and a sulfur atom; to be specific, it includes, for example, imidazolyl, pyridyl, quinolyl, furyl, thienyl, piperidyl, morpholino, benzoxazolyl, benzimidazolyl, benzothiazolyl and the like), a silyl group (it has preferably 3 to 40 carbon atoms, more preferably 3 to 30 carbon atoms and particularly preferably 3 to 24 carbon atoms and includes, for example, trimethylsilyl, triphenylsilyl and the like) and the like. The above substituents may be further substituted.

The substituents R^{1c}, R^{2c} and R^{3c} in Formula (2c) are selected preferably from an alkyl group and an aryl group.

Publicly known various amine derivatives such as amine derivatives shown in the paragraphs of [0079] to [0083] of Japanese Patent Application Laid-Open No. 324678/2002 can be given as the specific examples of the amine derivative represented by Formula (2c) used for the organic EL device of the present invention. The representative specific examples thereof are shown below.

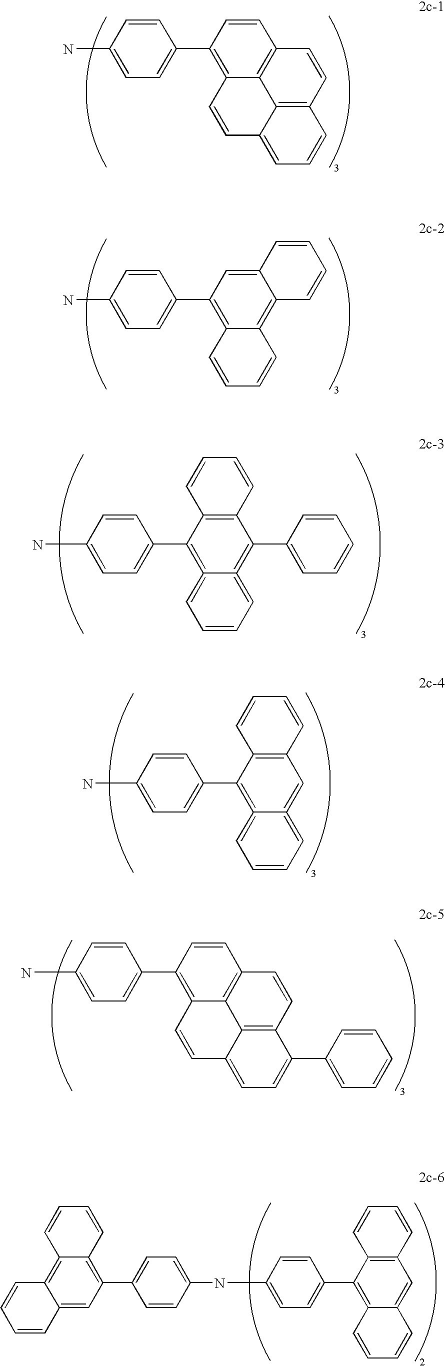

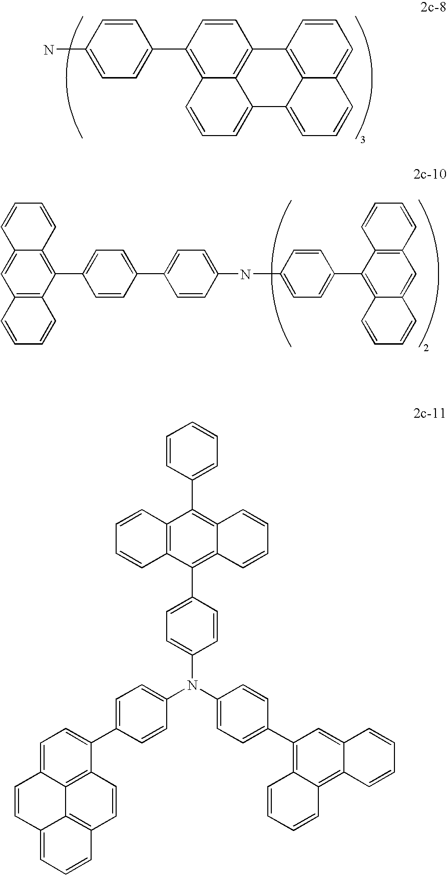

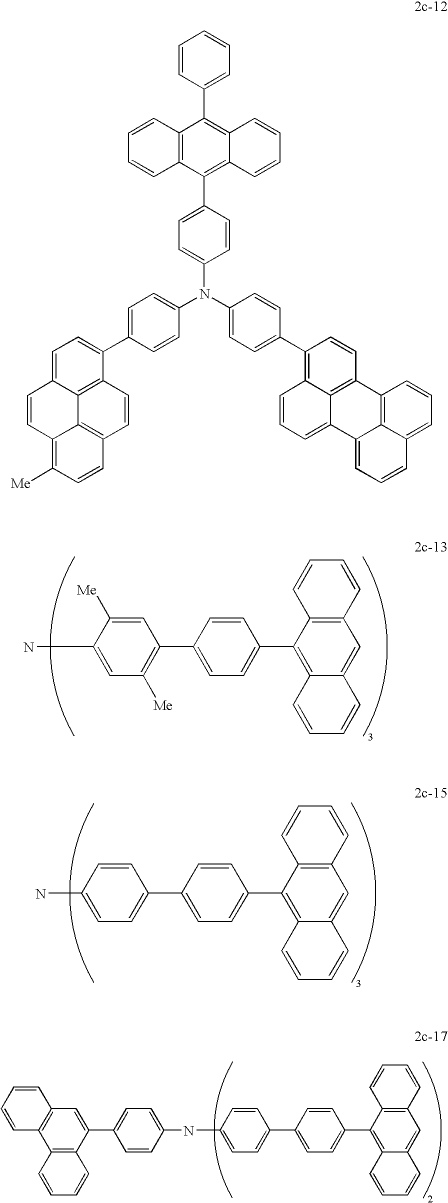

### Formula (2d)

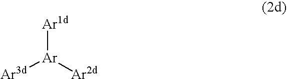

(in Formula (2d), Ar^{1d}, Ar^{2d} and Ar^{3d} each represent independently a substituted or non-substituted aryl group having 6 to 50 ring carbon atoms; the aryl group described above may be substituted with at least one substituent; at least one of Ar^{1d}, Ar^{2d}, Ar^{3d} and the substituents present on the above aryl groups has a fused ring aryl structure having 10 to 20 ring carbon atoms or a fused ring heteroaryl structure having 6 to 20 ring carbon atoms; and Ar represents a trivalent group derived from an aromatic ring or an aromatic heterocycle).

The aryl group having 6 to 50 ring carbon atoms represented by Ar^{1d}, Ar^{2d} and Ar^{3d} in Formula (2d) has preferably 6 to 30 ring carbon atoms, more preferably 6 to 20 ring carbon atoms and further preferably 6 to 16 ring carbon atoms. The aryl group includes phenyl, naphthyl, anthryl, phenanthrenyl, pyrenyl, perylenyl, fluorenyl, biphenylyl, terphenylyl, rubrenyl, chrysenyl, triphenylenyl, benzanthryl, benzophenanthrenyl, diphenylanthryl and the like. The above aryl groups may further have substituents.

The substituents present on the aryl groups include, for example, an alkyl group (it has preferably 1 to 30 carbon atoms, more preferably 1 to 20 carbon atoms and particularly preferably 1 to 10 carbon atoms and includes, for example, methyl, ethyl, isopropyl, t-butyl, n-octyl, n-decyl, n-hexadecyl, cyclopropyl, cyclopentyl, cyclohexyl and the like), an alkenyl group (it has preferably 2 to 30 carbon atoms, more preferably 2 to 20 carbon atoms and particularly preferably 2 to 10 carbon atoms and includes, for example, vinyl, allyl, 2-butenyl, 3-pentenyl and the like), an alkynyl group (it has preferably 2 to 30 carbon atoms, more preferably 2 to 20 carbon atoms and particularly preferably 2 to 10 carbon atoms and includes, for example, propargyl, 3-pentynyl and the like), an aryl group (it has preferably 6 to 30 carbon atoms, more preferably 6 to 20 carbon atoms and particularly preferably 6 to 12 carbon atoms and includes, for example, phenyl, p-methylphenyl, naphthyl, anthranyl and the like), an amino group (it has preferably 0 to 30 carbon atoms, more preferably 0 to 20 carbon atoms and particularly preferably 0 to 10 carbon atoms and includes, for example, amino, methylamino, dimethylamino, diethylamino, dibenzylamino, diphenylamino, ditolylamino and the like), an alkoxy group (it has preferably 1 to 30 carbon atoms, more preferably 1 to 20 carbon atoms and particularly preferably 1 to 10 carbon atoms and includes, for example, methoxy, ethoxy, butoxy, 2-ethylhexyloxy and the like), an aryloxy group (it has preferably 6 to 30 carbon atoms, more preferably 6 to 20 carbon atoms and particularly preferably 6 to 12 carbon atoms and includes, for example, phenyloxy, 1-naphthyloxy, 2-naphthyloxy and the like), a heteroaryloxy group (it has preferably 1 to 30 carbon atoms, more preferably 1 to 20 carbon atoms and particularly preferably 1 to 12 carbon atoms and includes, for example, pyridyloxy, pyrazyloxy, pyrimidyloxy, quinolyloxy and the like), an acyl group (it has preferably 1 to 30 carbon atoms, more preferably 1 to 20 carbon atoms and particularly preferably 1 to 12 carbon atoms and includes, for example, acetyl, benzoyl, formyl, pivaloyl and the like), an alkoxycarbonyl group (it has preferably 2 to 30 carbon atoms, more preferably 2 to 20 carbon atoms and particularly preferably 2 to 12 carbon atoms and includes, for example, methoxycarbonyl, ethoxycarbonyl and the like), an aryloxycarbonyl group (it has preferably 7 to 30 carbon atoms, more preferably 7 to 20 carbon atoms and particularly preferably 7 to 12 carbon atoms and includes, for example, phenyloxycarbonyl and the like), an acyloxy group (it has preferably 2 to 30 carbon atoms, more preferably 2 to 20 carbon atoms and particularly preferably 2 to 10 carbon atoms and includes, for example, acetoxy, benzoyloxy and the like), an acylamino group (it has preferably 2 to 30 carbon atoms, more preferably 2 to 20 carbon atoms and particularly preferably 2 to 10 carbon atoms and includes, for example, acetylamino, benzoylamino and the like), an alkoxycarbonylamino group (it has preferably 2 to 30 carbon atoms, more preferably 2 to 20 carbon atoms and particularly preferably 2 to 12 carbon atoms and includes, for example, methoxycarbonylamino and the like), an aryloxycarbonylamino group (it has preferably 7 to 30 carbon atoms, more preferably 7 to 20 carbon atoms and particularly preferably 7 to 12 carbon atoms and includes, for example, phenyloxycarbonylamino and the like), a sulfonylamino group (it has preferably 1 to 30 carbon atoms, more preferably 1 to 20 carbon atoms and particularly preferably 1 to 12 carbon atoms and includes, for example, methanesulfonylamino, benzenesulfonylamino and the like), a sulfamoyl group (it has preferably 0 to 30 carbon atoms, more preferably 0 to 20 carbon atoms and particularly preferably 0 to 12 carbon atoms and includes, for example, sulfamoyl, methylsulfamoyl, dimethylsulfamoyl, phenylsulfamoyl and the like), a carbamoyl group (it has preferably 1 to 30 carbon atoms, more preferably 1 to 20 carbon atoms and particularly preferably 1 to 12 carbon atoms and includes, for example, carbamoyl, methylcarbamoyl, diethylcarbamoyl, phenylcarbamoyl and the like), an alkylthio group (it has preferably 1 to 30 carbon atoms, more preferably 1 to 20 carbon atoms and particularly preferably 1 to 12 carbon atoms and includes, for example, methylthio, ethylthio and the like), an arylthio group (it has preferably 6 to 30 carbon atoms, more preferably 6 to 20 carbon atoms and particularly preferably 6 to 12 carbon atoms and includes, for example, phenylthio and the like), a heteroarylthio group (it has preferably 1 to 30 carbon atoms, more preferably 1 to 20 carbon atoms and particularly preferably 1 to 12 carbon atoms and includes, for example, pyridylthio, 2-benzimidazolylthio, 2-benzoxazolylthio, 2-benzothiazolylthio and the like), a sulfonyl group (it has preferably 1 to 30 carbon atoms, more preferably 1 to 20 carbon atoms and particularly preferably 1 to 12 carbon atoms and includes, for example, mesyl, tosyl and the like), a sulfinyl group (it has preferably 1 to 30 carbon atoms, more preferably 1 to 20 carbon atoms and particularly preferably 1 to 12 carbon atoms and includes, for example, methanesulfinyl, benznesulfinyl and the like), a ureido group (it has preferably 1 to 30 carbon atoms, more preferably 1 to 20 carbon atoms and particularly preferably 1 to 12 carbon atoms and includes, for example, ureido, methylureido, phenylureido and the like), a phosphoric amide group (it has preferably 1 to 30 carbon atoms, more preferably 1 to 20 carbon atoms and particularly preferably 1 to 12 carbon atoms and includes, for example, diethylphosphoric amide, phenylphosphoric amide and the like), a hydroxy group, a mercapto group, a halogen atom (including, for example, a fluorine atom, a chlorine atom, a bromine atom and an iodine atom), a cyano group, a sulfo group, a carboxy group, a nitro group, a hydroxamic acid group, a sulfino group, a hydrazino group, an imino group, a heterocyclic group (it has preferably 1 to 30 carbon atoms, more preferably 1 to 12 carbon atoms, and a hetero atom includes, for example, a nitrogen atom, an oxygen atom and a sulfur atom; to be specific, it includes, for example, imidazolyl, pyridyl, quinolyl, furyl, thienyl, piperidyl, morpholino, benzoxazolyl, benzimidazolyl, benzothiazolyl, carbazolyl, azepinyl and the like), a silyl group (it has preferably 3 to 40 carbon atoms, more preferably 3 to 30 carbon atoms and particularly preferably 3 to 24 carbon atoms and includes, for example, trimethylsilyl, triphenylsilyl and the like) and the like. The above substituents may be further substituted.

The fused ring aryl structure having 10 to 20 ring carbon atoms present on at least one of Ar^{1d}, Ar^{2d} and Ar^{3d} in Formula (2d) and the substituents present on the above aryl groups includes a naphthalene structure, an anthracene structure, a phenanthrene structure, a pyrene structure, a perylene structure and the like, and it is preferably a naphthalene structure, an anthracene structure, a pyrene structure and a phenanthrene structure, more preferably a phenanthrene structure and an aryl structure comprising 4 or more rings and particularly preferably a pyrene structure.

The fused ring heteroaryl structure having 6 to 20 ring carbon atoms present on at least one of Ar^{1d}, Ar^{2d} and Ar^{3d} in Formula (2d) and the substituents present on the above aryl groups includes a quinoline structure, a quinoxaline structure, a quinazoline structure, an acridine structure, a phenanthridine structure, a phthalazine structure, a phenanthroline structure and the like, and it is preferably a quinoline structure, a quinoxaline structure, a quinazoline structure, a phthalazine structure and a phenanthroline structure.

The trivalent group derived from an aromatic ring represented by Ar in Formula (2d) has preferably 6 to 30 carbon atoms, more preferably 6 to 20 carbon atoms and further preferably 6 to 16 carbon atoms. To be specific, it includes trivalent groups derived from benzene, naphthalene, anthracene, phenanthrene, pyrene and triphenylene.

The trivalent group derived from an aromatic heterocycle represented by Ar in Formula (2d) contains preferably an atom selected from a nitrogen atom, a sulfur atom and an oxygen atom as a hetero atom, and it contains more preferably a nitrogen atom. It has preferably 2 to 30 carbon atoms, more preferably 3 to 20 carbon atoms and further preferably 3 to 16 carbon atoms. To be specific, it includes trivalent groups derived from pyridine, pyrazine, thiopyran, quinoline, quinoxaline and triazine. The trivalent groups derived from the above aromatic rings and aromatic heterocycles may have substituents. The substituents include groups shown by the substituents on the aryl groups represented by the substituents Ar^{1d}, Ar^{2d} and Ar^{3d}. Ar includes preferably benzenetriyl, naphthalenetriyl, anthracenetriyl, pyrenetriyl or a trivalent group derived from triphenylene, more preferably benzenetriyl and further preferably non-substituted (Ar^{1d}, Ar^{2d} and Ar^{3d} are substituted) benzenetriyl or alkyl-substituted benzenetriyl.

Publicly known various benzene derivatives such as benzene derivatives shown in the paragraphs of [0079] to [0083] of Japanese Patent Application Laid-Open No. 324678/2002 can be given as the specific examples of the benzene derivative represented by Formula (2d) used for the organic EL device of the present invention. The representative specific examples thereof are shown below.

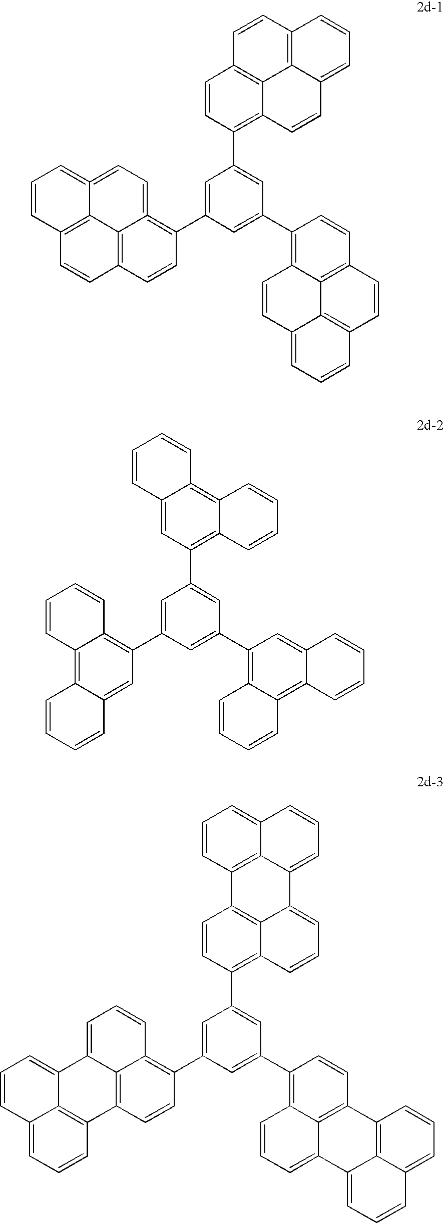

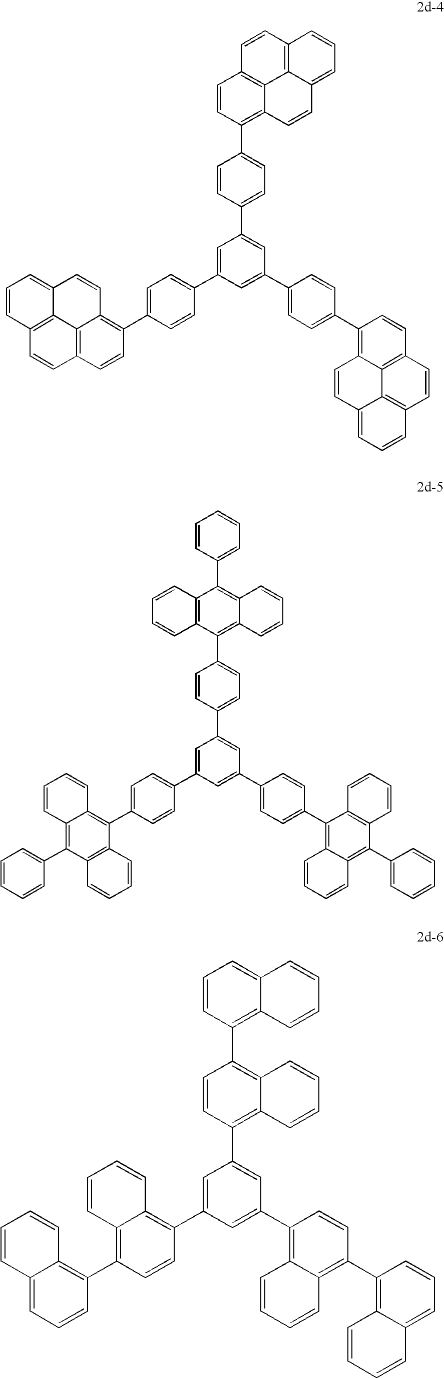

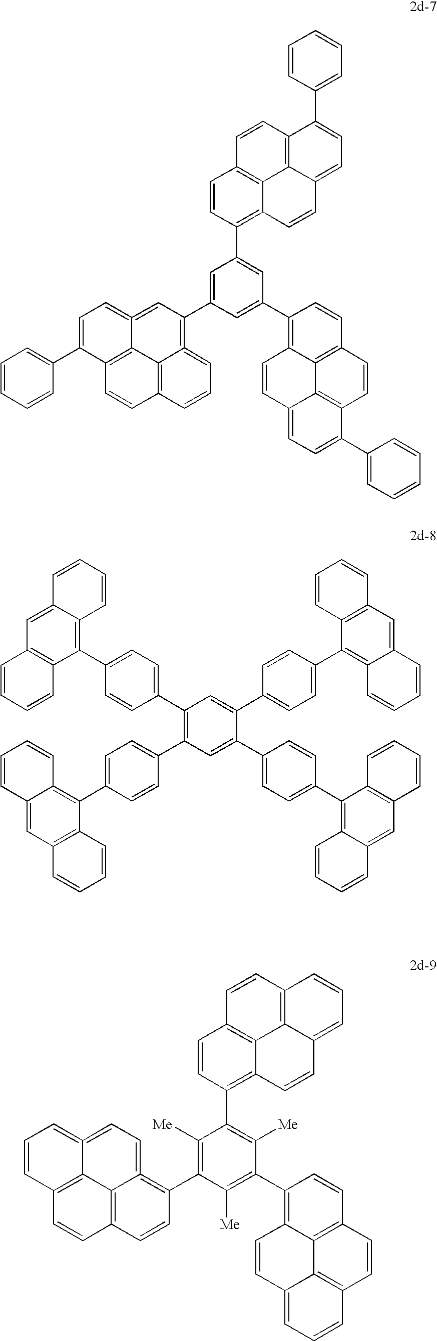

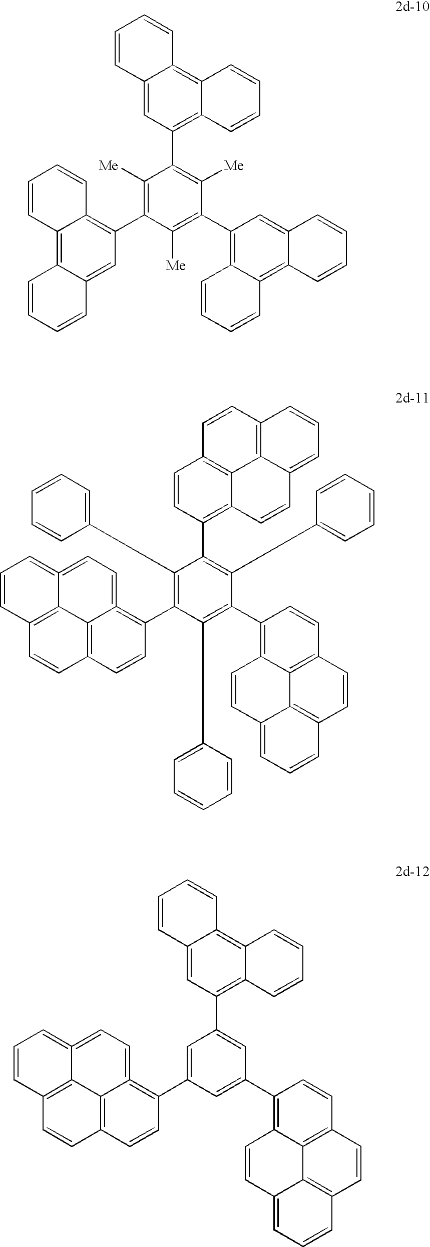

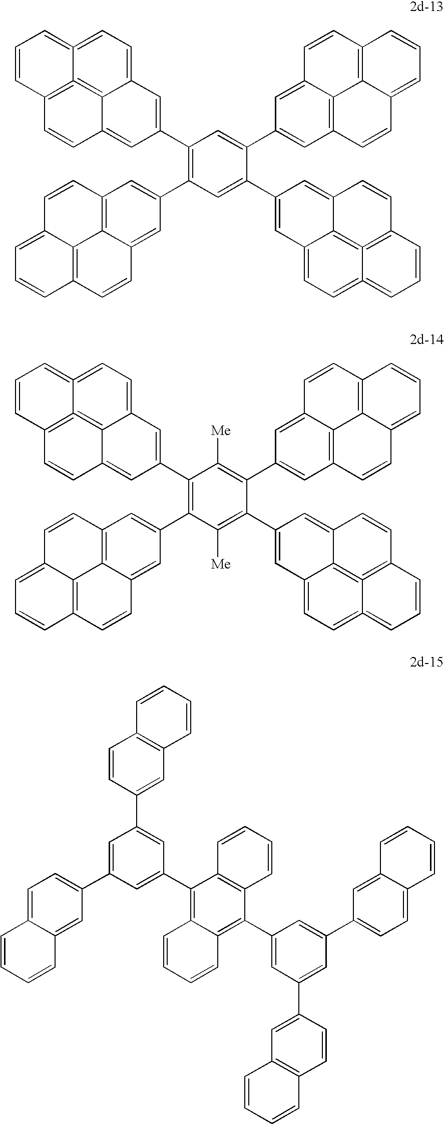

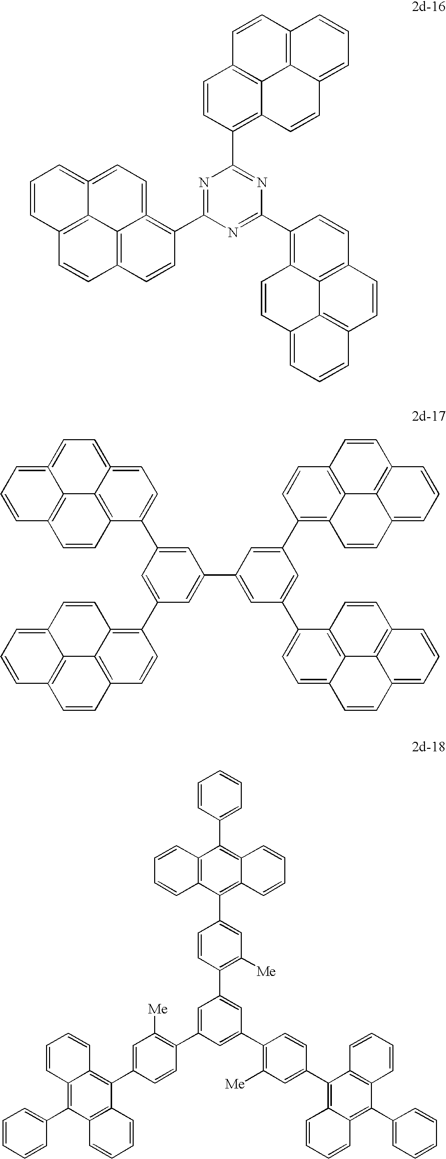

In the organic EL device of the present invention, dry film forming methods such as vacuum vapor deposition, a molecular beam epitaxy method (MBE method), sputtering, plasma and ion plating and coating methods such as spin coating, dipping, casting, bar coating, roll coating, flow coating and ink jet each using a solution prepared by dissolving the compound in a solvent can be applied to the formation of the respective organic layers such as the light emitting layer and the like. The film thicknesses of the respective layers in the organic compound layer shall not specifically be restricted but have to be set to suitable film thicknesses. In general, if the film thicknesses are too small, pinholes and the like are produced, and when an electric field is applied, no satisfactory light emitting luminance is likely to be obtained. On the other hand, if they are too large, high voltage has to be applied in order to obtain a constant light output, and the efficiency is deteriorated. Accordingly, they fall usually in a range of suitably 5 nm to 10 μm, preferably 10 nm to 0.2 μm.

In the case of a wet film forming method, the materials forming the respective layers are dissolved or dispersed in suitable solvents to prepare the light emitting organic solutions, and the thin films are formed therefrom. The solvents may be any ones. The solvents include, for example, halogen base hydrocarbon solvents such as dichloromethane, dichloroethane, chloroform, carbon tetrachloride, tetrachloroethane, trichloroethane, chlorobenzene, dichlorobenzene, chlorotoluene, trifluorotoluene and the like, ether base solvents such as dibutyl ether, tetrahydrofuran, tetrahydropyran, dioxane, anisole, dimethoxyethane and the like, alcohol base solvents such as methanol, ethanol, propanol, isopropanol, butanol, pentanol, hexanol, cyclohexanol, methyl cellosolve, ethyl cellosolve, ethylene glycol and the like, ketone base solvents such as acetone, methyl ethyl ketone, diethyl ketone, 2-hexanone, methyl isobutyl ketone, 2-heptanone, 4-heptanone, diisobutyl ketone, acetonylacetone, isophorone, cyclohexanone, methylhexanone, acetophenone and the like, hydrocarbon base solvents such as benzene, toluene, xylene, ethylbenzene, hexane, cyclohexane, octane, decane, tetralin and the like, ester base solvents such as ethyl acetate, butyl acetate, amyl acetate and the like, chain carbonic ester base solvents such as dimethyl carbonate, methyl ethyl carbonate, diethyl carbonate and the like and cyclic carbonic ester base solvents such as ethylene carbonate, propylene carbonate and the like. Among them, the hydrocarbon base solvents and the ether base solvents such as toluene, dioxane and the like are preferred. The above solvents may be used alone or in a mixture of two or more kinds thereof. Solvents which can be used shall not be restricted to them.

In any organic thin film layers, suitable resins and additives may be used in order to improve a film forming property and prevent pinholes from being formed on the films. The resins which can be used include insulating resins such as polystyrene, polycarbonate, polyallylate, polyester, polyamide, polyurethane, polysulfone, polymethyl methacrylate, polymethyl acrylate, cellulose and the like and copolymers thereof, photoconductive resins such as poly-N-vinylcarbazole, polysilane and the like and conductive resins such as polyaniline, polythiophene, polypyrrole and the like. The additives include antioxidants, UV absorbers, plasticizers and the like.

For the purpose of enhancing a stability of the organic EL device obtained according to the present invention toward temperature, humidity, atmosphere and the like, a protective layer can be provided on the surface of the device or the whole part of the device can be protected by silicone oil, resins and the like.

In the organic EL device of the present invention, the dibenzo[c,g]triphenylene derivative of the present invention is contained in the light emitting layer in a proportion of preferably 0.01 to 20% by weight, more preferably 0.5 to 20% by weight.

In the organic EL device of the present invention, a layer selected from a chalcogenide layer, a halogenated metal layer and a metal oxide layer is preferably provided on a surface of at least one of a pair of the electrodes.

### Constitution of the Organic EL Device:

The device constitution of the organic EL device of the present invention shall be explained below.

### (1) Constitution of the Organic EL Device

The representative device constitution of the organic EL device of the present invention includes constitutions such as:
(1) Anode/light emitting layer/cathode
(2) Anode/hole injecting layer/light emitting layer/cathode
(3) Anode/light emitting layer/electron injecting layer/cathode
(4) Anode/hole injecting layer/light emitting layer/electron injecting layer/cathode
(5) Anode/organic semiconductor layer/light emitting layer/cathode
(6) Anode/organic semiconductor layer/electron barrier layer/light emitting layer/cathode
(7) Anode/organic semiconductor layer/light emitting layer/adhesion improving layer/cathode
(8) Anode/hole injecting layer/hole transporting layer/light emitting layer/electron injecting layer/cathode
(9) anode/insulating layer/light emitting layer/insulating layer/cathode,
(10) anode/inorganic semiconductor layer/insulating layer/light emitting layer/insulating layer/cathode
(11) anode/organic semiconductor layer/insulating layer/light emitting layer/insulating layer/cathode,
(12) anode/insulating layer/hole injecting layer/hole transporting layer/light emitting layer/insulating layer/cathode and
(13) anode/insulating layer/hole injecting layer/hole transporting layer/light emitting layer/electron injecting layer/cathode

Among them, usually the constitution of (8) is preferably used.

The compound of the present invention may be used in any organic layers described above and is preferably contained in the light emitting zone or the hole transporting zone among the above constitutional elements. The content is selected from 30 to 100 mole %.

### (2) Light Transmitting Substrate

The organic EL device of the present invention is prepared on a light transmitting substrate. The light transmitting substrate referred to in this case is a substrate for supporting the organic EL device, and it is preferably a flat substrate in which light in a visible region of 400 to 700 nm has a transmittance of 50% or more.

To be specific, it includes a glass plate, a polymer plate and the like. In particular, the glass plate includes soda lime glass, barium-strontium-containing glass, lead glass, aluminosilicate glass, borosilicate glass, barium borosilicate glass, quartz and the like. The polymer plate includes polycarbonate resin, acrylic resin, polyethylene terephthalate resin, polyether sulfide resin, polysulfone resin and the like.

### (3) Anode

An anode in the organic EL device of the present invention assumes a role of injecting a hole into the hole transporting layer or the light emitting layer, and it is effective to provide the anode with a work function of 4.5 eV or more. The specific examples of a material for the anode used in the present invention include indium tin oxide alloy (ITO), tin oxide (NESA), gold, silver, platinum, copper and the like. A material having a small work function is preferred as the anode for the purpose of injecting an electron into the electron transporting layer or the light emitting layer.

The anode can be prepared by forming a thin film from the above electrode substances by a method such as a vapor deposition method, a sputtering method and the like.

When light emitted from the light emitting layer is taken out from the anode, a transmittance of light in the anode based on light emitted is preferably larger than 10%. A sheet resistance of the anode is preferably several hundred Ω/□ or less. A film thickness of the anode is selected, though depending on the material, in a range of usually 10 nm to 1 μm, preferably 10 to 200 nm.

### (4) Light Emitting Layer

The light emitting layer in the organic EL device has the following functions in combination. That is,

(1) injecting function: a function in which a hole can be injected from an anode or a hole injecting layer and in which an electron can be injected from a cathode or an electron injecting layer in applying an electric field,
(2) transporting function: a function in which a charge (electron and hole) injected is transferred by virtue of a force of an electric field and
(3) light emitting function: a function in which a field for recombination of an electron and a hole is provided and in which this is connected to light emission. Provided that a difference may be present between an easiness in injection of a hole and an easiness in injection of an electron and that a difference may be present in a transporting ability shown by the mobilities of a hole and an electron, and any one of the charges is preferably transferred.

A publicly known method such as, for example, a vapor deposition method, a spin coating method, an LB method, a dipping method, a spin coating method, a casting method, a bar coating method, a roll coating method and the like can be applied as a method for forming the above light emitting layer. In particular, the light emitting layer is preferably a molecular deposit film.

In this regard, the molecular deposit film means a thin film formed by depositing a material compound staying in a gas phase state and a film formed by solidifying a material compound staying in a solution state or a liquid phase state, and the above molecular deposit film can usually be distinguished from a thin film formed by an LB method (molecular accumulation film) by a difference in an aggregation structure and a higher order structure and a functional difference originating in it.

Further, as disclosed in Japanese Patent Application Laid-Open No. 51781/1982, the light emitting layer can be formed as well by dissolving a binding agent such as a resin and the material compound in a solvent to prepare a solution and then coating the solution by a spin coating method and the like to form a thin film.

In the present invention, other publicly known light emitting materials excluding the light emitting material comprising the compound containing the dibenzo[c, g]triphenylene derivative of the present invention and the fused ring-containing compound may be added, if necessary, to the light emitting layer as long as the object of the present invention is not damaged. Further, a light emitting layer containing a different publicly known light emitting material may be laminated on the light emitting layer containing the light emitting material of the present invention.

Further, a film thickness of the light emitting layer is preferably 5 to 50 nm, more preferably 7 to 50 nm and most preferably 10 to 50 nm. If it is less than 5 nm, it is likely to be difficult to form the light emitting layer and control the chromaticity. On the other hand, if it exceeds 50 nm, the operating voltage is likely to go up.

### (5) Hole Injecting and Transporting Layer (Hole Transporting Zone)

The hole injecting and transporting layer is a layer for assisting injection of a hole into the light emitting layer to transport it to the light emitting region, and it has a large hole mobility and shows usually as small ionization energy as 5.6 eV or less. A material which transports a hole to the light emitting layer by a lower electric field strength is preferred for the above hole injecting and transporting layer, and more preferred is a material in which a mobility of a hole is at least 10⁻⁴ cm²/V·second in applying an electric field of, for example, 10⁻⁴ to 10⁶ V/cm.

When the dibenzo[c,g]triphenylene derivative of the present invention is used in the hole transporting zone, the hole injecting and transporting layers may be formed from the dibenzo[c,g]triphenylene derivative of the present invention alone or it may be used in a mixture with other materials.

The materials for forming the hole injecting and transporting layer in a mixture with the dibenzo[c,g]triphenylene derivative of the present invention shall not specifically be restricted as long as they have the preferred properties described above, and capable of being used are optional materials selected from materials which have so far conventionally been used as charge transporting materials for holes in photoconductive materials and publicly known materials which are used for a hole injecting and transporting layer in an organic EL device.

The specific examples thereof include triazole derivatives (refer to U.S. Pat. No. 3,112,197 and the like), oxadiazole derivatives (refer to U.S. Pat. No. 3,189,447 and the like), imidazole derivatives (refer to Japanese Patent Publication No. 16096/1962 and the like), polyarylalkane derivatives (refer to U.S. Pat. No. 3,615,402, ditto U.S. Pat. No. 3,820,989 and ditto U.S. Pat. No. 3,542,544, Japanese Patent Publication No. 555/1970 and ditto No. 10983/1976 and Japanese Patent Application Laid-Open No. 93224/1976, ditto No. 17105/1980, ditto No. 4148/1981, ditto No. 108667/1980, ditto No. 156953/1980 and ditto No. 36656/1981 and the like), pyrazoline derivatives and pyrazolone derivatives (refer to U.S. Pat. No. 3,180,729 and ditto U.S. Pat. No. 4,278,746 and Japanese Patent Application Laid-Open No. 88064/1980, ditto No. 88065/1980, ditto No. 105537/1974, ditto No. 51086/1980, ditto No. 80051/1981, ditto No. 88141/1981, ditto No. 45545/1982, ditto No. 112637/1979 and ditto No. 74546/1980 and the like), phenylenediamine derivatives (refer to U.S. Pat. No. 3,615,404, Japanese Patent Publication No. 10105/1976, ditto No. 3712/1971, ditto No. 25336/1972 and ditto 119925/1979 and the like), arylamine derivatives (refer to U.S. Pat. No. 3,567,450, ditto U.S. Pat. No. 3,240,597, ditto U.S. Pat. No. 3,658,520, ditto U.S. Pat. No. 4,232,103, ditto U.S. Pat. No. 4,175,961 and ditto U.S. Pat. No. 4,012,376, Japanese Patent Publication No. 35702/1974 and ditto No. 27577/1964, Japanese Patent Application Laid-Open No. 144250/1980, ditto No. 119132/1981 and ditto No. 22437/1981 and German Patent No. 1,110,518 and the like), amino-substituted chalcone derivatives (refer to U.S. Pat. No. 3,526,501 and the like), oxazole derivatives (disclosed in U.S. Pat. No. 3,257,203 and the like), styrylanthracene derivatives (refer to Japanese Patent Application Laid-Open No. 46234/1981 and the like), fluorenone derivatives (refer to Japanese Patent Application Laid-Open No. 110837/1979 and the like), hydrazone derivatives (refer to U.S. Pat. No. 3,717,462, Japanese Patent Application Laid-Open No. 59143/1979, ditto No. 52063/1980, ditto No. 52064/1980, ditto No. 46760/1980, ditto No. 11350/1982 and ditto No. 148749/1982, Japanese Patent Application Laid-Open No. 311591/1990 and the like), stilbene derivatives (refer to Japanese Patent Application Laid-Open No. 210363/1986, ditto No. 228451/1986, ditto No. 14642/1986, ditto No. 72255/1986, ditto No. 47646/1987, ditto No. 36674/1987, ditto No. 10652/1987, ditto No. 30255/1987, ditto No. 93455/1985, ditto No. 94462/1985, ditto No. 174749/1985 and ditto No. 175052/1985 and the like), silazane derivatives (U.S. Pat. No. 4,950,950), polysilane base (Japanese Patent Application Laid-Open No. 204996/1990) and aniline base copolymers (Japanese Patent Application Laid-Open No. 282263/1990).

The compounds described above can be used as the material for the hole injecting and transporting layer, and preferably used are porphyrin compounds (disclosed in Japanese Patent Application Laid-Open No. 295695/1988 and the like) and aromatic tertiary amine compounds and styrylamine compounds (refer to U.S. Pat. No. 4,127,412 and Japanese Patent Application Laid-Open No. 27033/1978, ditto No. 58445/1979, ditto No. 79450/1980, ditto No. 144250/1980, ditto No. 119132/1981, ditto No. 295558/1986, ditto No. 98353/1986 and ditto No. 295695/1988 and the like), and the aromatic tertiary amine compounds are particularly preferably used.

Further, capable of being given are compounds having two fused aromatic rings in a molecule described in U.S. Pat. No. 5,061,569, for example, 4,4′-bis(N-(1-naphthyl)-N-phenylamino)biphenyl (hereinafter abbreviated as NPD) and 4,4′,4″-tris(N-(3-methylphenyl)-N-phenylamino)triphenylamine (hereinafter abbreviated as MTDATA) in which three triphenylamine units are connected in the form of a star burst type disclosed in Japanese Patent Application Laid-Open No. 308688/1992.

Further, inorganic compounds such as p type Si, p type SiC and the like can also be used as the material for the hole injecting layer in addition to the aromatic dimethylidene base compounds described above shown as the material for the light emitting layer.

The hole injecting and transporting layer can be formed by making a thin film from the compound described above by a publicly known method such as, for example, a vacuum vapor deposition method, a spin coating method, a casting method, an LB method and the like. A film thickness of the hole injecting and transporting layer shall not specifically be restricted, and it is usually 5 nm to 5 μm.

### (6) Electron Injecting Layer

The electron injecting layer is a layer for assisting injection of an electron into the light emitting layer, and it has a large electron mobility. Also, the adhesion improving layer is a layer comprising particularly a material having a good adhesive property with the cathode in the above electron injecting layer. Materials used for the electron injecting layer are suitably metal complexes of 8-hydroxyquinoline or derivatives thereof.

The specific examples of the metal complexes of 8-hydroxyquinoline or the derivatives thereof include metal chelate oxynoid compounds containing chelates of oxine (in general, 8-quinolinol or 8-hydroxyquinoline).

For example, Alq described in the item of the light emitting material can be used for the electron injecting layer.

On the other hand, the oxadiazole derivative includes an electron transfer compound represented by the following

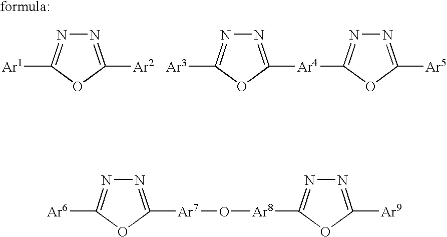

(wherein Ar¹, Ar², Ar³, Ar⁵, Ar⁶ and Ar⁹ represent a substituted or non-substituted aryl group, and they may be the same as or different from each other; Ar⁴, Ar⁷ and Ar⁸ represent a substituted or non-substituted arylene group, and they may be the same as or different from each other).

In this regard, the aryl group includes phenyl, biphenyl, anthracenyl, perylenyl and pyrenyl. The arylene group includes phenylene, naphthylene, biphenylene, anthracenylene, perylenylene and pyrenylene. The substituent is preferably an alkyl group having 1 to 10 carbon atoms, an alkoxy group having 1 to 10 carbon atoms or a cyano group. The above electron transfer compound is preferably a compound having a thin film forming property.

The following compounds can be given as the specific examples of the electron transfer compound.

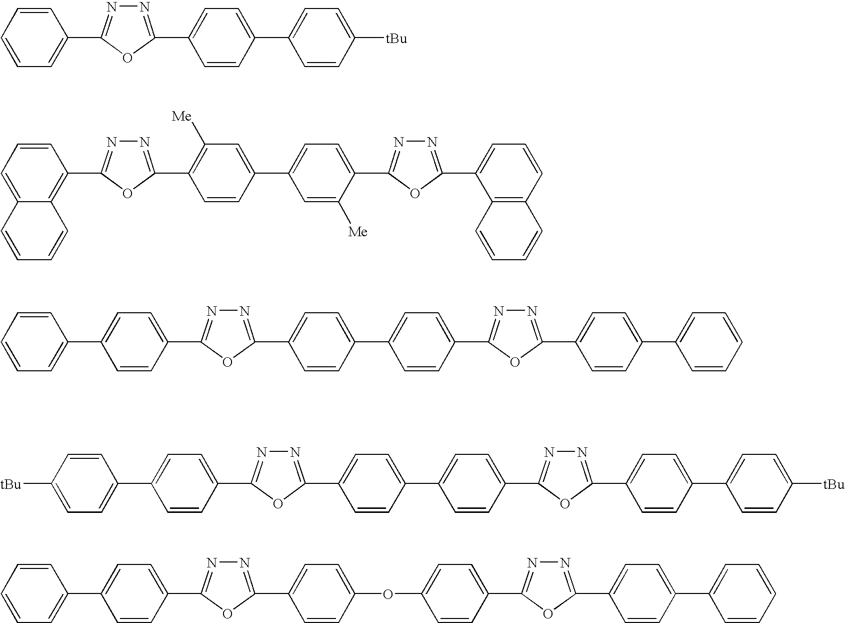

The preferred mode of the organic EL device of the present invention includes a device containing a reducing dopant in the region which transports an electron or an interfacial region between the cathode and the organic layer. In this case, the reducing dopant is defined by a substance which can reduce an electron transporting compound. Accordingly, various compounds can be used as long as they have a certain reducing property, and capable of being suitably used is at least one substance selected from the group consisting of, for example, alkali metals, alkaline earth metals, rare earth metals, oxides of alkali metals, halides of alkali metals, oxides of alkaline earth metals, halides of alkaline earth metals, oxides of rare earth metals or halides of rare earth metals, organic complexes of alkali metals, organic complexes of alkaline earth metals and organic complexes of rare earth metals.

To be more specific, the preferred reducing dopant includes at least one alkali metal selected from the group consisting of Li (work function: 2.9 eV), Na (work function: 2.36 eV), K (work function: 2.28 eV), Rb (work function: 2.16 eV) and Cs (work function: 1.95 eV) and at least one alkaline earth metal selected from the group consisting of Ca (work function: 2.9 eV), Sr (work function: 2.0 to 2.5 eV) and Ba (work function: 2.52 eV), and the compounds having a work function of 2.9 eV or less are particularly preferred. Among them, the more preferred reducing dopant is at least one alkali metal selected from the group consisting of K, Rb and Cs, and it is more preferably Rb or Cs. It is most preferably Cs. The above alkali metals have a particularly high reducing ability, and addition of a relatively small amount thereof to the electron injecting zone makes it possible to raise a light emitting luminance in the organic EL device and extend a lifetime thereof. The combination of two or more kinds of the above alkali metals is preferred as the reducing dopant having a work function of 2.9 eV or less, and particularly preferred is the combination containing Cs, for example, the combination of Cs with Na, Cs with K, Cs with Rb or Cs with Na and K. Containing Cs in combination makes it possible to efficiently exhibit the reducing ability, and addition thereof to the electron injecting zone makes it possible to enhance a light-emitting luminance in the organic EL device and extend a lifetime thereof.

In the present invention, an electron injecting layer constituted from an insulator and a semiconductor may further be provided between the cathode and the organic layer. In this case, an electric current can effectively be prevented from leaking to enhance the electron injecting property. Preferably used as the above insulator is at least one metal compound selected from the group consisting of alkali metal chalcogenides, alkaline earth metal chalcogenides, halides of alkali metals and halides of alkaline earth metals. If the electron injecting layer is constituted from the above alkali metal chalcogenides and the like, it is preferred from the viewpoint that the electron injecting property can further be enhanced. To be specific, the preferred alkali metal chalcogenides include, for example, Li₂O, K₂O, Na₂S, Na₂Se and Na₂O, and the preferred alkaline earth metal chalcogenides include, for example, CaO, BaO, SrO, BeO, BaS and CaSe. Also, the preferred halides of alkali metals include, for example, LiF, NaF, KF, LiCl, KCl and NaCl. Further, the preferred halides of alkaline earth metals include, for example, fluorides such as CaF₂, BaF₂, SrF₂, MgF₂ and BeF₂ and halides other than the fluorides.

The semiconductor constituting the electron transporting layer includes a single kind of oxides, nitrides or nitride oxides containing at least one element of Ba, Ca, Sr, Yb, Al, Ga, In, Li, Na, Cd, Mg, Si, Ta, Sb and Zn or combinations of two or more kinds thereof. The inorganic compound constituting the electron transporting layer is preferably a crystallite or amorphous insulating thin film. If the electron transporting layer is constituted from the above insulating thin film, the more homogeneous thin film is formed, and therefore pixel defects such as dark spots and the like can be reduced. The above inorganic compound includes the alkali metal chalcogenides, the alkaline earth metal chalcogenides, the halides of alkali metals and the halides of alkaline earth metals each described above.

### (7) Cathode

Cathodes prepared by using metals, alloys, electroconductive compounds and mixtures thereof each having a small work function (4 eV or less) for electrode materials are used as the cathode in order to inject electrons into the electron injecting and transporting layer or the light emitting layer. The specific examples of the above electrode materials include sodium, sodium.potassium alloys, magnesium, lithium, magnesium.silver alloys, aluminum/aluminum oxide, aluminum.lithium alloys, indium, rare earth metals and the like.

The above cathode can be prepared by forming a thin film from the above electrode materials by a method such as vapor deposition, sputtering and the like.

In this respect, when light emitted from the light emitting layer is taken out from the cathode, a transmittance of the cathode based on light emitted is preferably larger than 10%.

A sheet resistance of the cathode is preferably several hundred Ω/□ or less, and a film thickness thereof is usually 10 nm to 1 μm, preferably 50 to 200 nm.

### (8) Insulating Layer

The organic EL device is liable to cause pixel defects by leakage and short circuit since an electric field is applied to a ultrathin film. In order to prevent the above matter, an insulating thin film layer is preferably interposed between a pair of the electrodes.

A material used for the insulating layer includes, for example, aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, vanadium oxide and the like, and mixtures and laminates thereof may be used as well.

### (9) Production Process for Organic EL Device

According to the materials and the forming methods which have been shown above as the examples, the anode, the light emitting layer, if necessary, the hole injecting layer and, if necessary, the electron injecting layer are formed, and further the cathode is formed, whereby the organic EL device can be prepared. Also, the organic EL device can be prepared as well in an order of from the cathode to the anode which is reverse to the order described above.

A preparation example of an organic EL device having a structure in which an anode/a hole injecting layer/a light emitting layer/an electron injecting layer/a cathode are provided in order on a light transmitting substrate shall be described below.

First, a thin film comprising an anode material is formed on a suitable light transmitting substrate by a method such as vapor deposition, sputtering and the like so that a film thickness falling in a range of 1 μm or less, preferably 10 to 200 nm is obtained, whereby an anode is prepared. Next, a hole injecting layer is provided on the above anode. The hole injecting layer can be formed, as described above, by a method such as a vacuum vapor deposition method, a spin coating method, a casting method, an LB method, an ink jet method and the like. The vacuum vapor deposition method is excellent because of the reasons that the homogeneous film is liable to be obtained and that pinholes are less liable to be produced. When forming the hole injecting layer by the vacuum vapor deposition method, the depositing conditions thereof are varied according to the compounds used (the materials for the hole injecting layer), the crystal structure of the targeted hole injecting layer and the recombination structure thereof, and in general, they are suitably selected preferably in the ranges of a deposition source temperature of 50 to 450° C., a vacuum degree of 10⁻⁷ to 10⁻³ Torr, a depositing speed of 0.01 to 50 nm/second, a substrate temperature of −50 to 300° C. and a film thickness of 5 nm to 5 μm.

Next, a light emitting layer can be formed on the hole injecting layer by forming a thin film from the desired organic light emitting material by a method such as a vacuum vapor deposition method, sputtering, a spin coating method, a casting method and the like, and it is formed preferably by the vacuum vapor deposition method because of the reasons that the homogeneous film is liable to be obtained and that pinholes are less liable to be produced. When forming the light emitting layer by the vacuum vapor deposition method, the depositing conditions thereof are varied according to the compounds used, and in general, they can be selected from the same condition ranges as in the hole injecting layer.

Next, an electron injecting layer is provided on the above light emitting layer. It is formed preferably by the vacuum vapor deposition method as is the case with the hole injecting layer and the light emitting layer since the homogeneous film has to be obtained. The depositing conditions thereof can be selected from the same condition ranges as in the hole injecting layer and the light emitting layer.

When using the vacuum vapor deposition method, the compound of the present invention can be codeposited together with the other materials, though varied depending on that it is added to any layer in the light emitting zone and the hole transporting zone. When using the spin coating method, it can be added in a mixture with the other materials.

Lastly, a cathode is laminated, whereby an organic EL device can be obtained.

The cathode is constituted from metal, and therefore the vapor deposition method and the sputtering method can be used. However, the vacuum vapor deposition method is preferred in order to protect the organic substance layer of the base from being damaged in making the film.

The organic EL device which has so far been described is preferably prepared serially from the anode up to the cathode after vacuuming once.

The forming methods of the respective layers in the organic EL device of the present invention shall not specifically be restricted, and forming methods carried out by a vacuum vapor deposition method, a spin coating method and the like which have so far publicly been known can be used. The organic thin film layer containing the compound represented by Formula (1) described above which is used for the organic EL device of the present invention can be formed by a publicly known method carried out by a vacuum vapor deposition method, a molecular beam epitaxy method (MBE method) and a coating method such as a dipping method, a spin coating method, a casting method, a bar coating method and a roll coating method each using a solution prepared by dissolving the compound in a solvent.

The film thicknesses of the respective organic layers in the organic EL device of the present invention shall not specifically be restricted, and in general, if the film thicknesses are too small, defects such as pinholes and the like are liable to be caused. On the other hand, if they are too large, high voltage has to be applied, and the efficiency is deteriorated, so that they fall usually in a range of preferably several nm to 1 μm.

When applying a DC voltage to the organic EL device, light emission can be observed by applying a voltage of 5 to 40 V setting a polarity of the anode to plus and that of the cathode to minus. An electric current does not flow by applying a voltage at a reverse polarity, and light emission is not caused at all. Further, when applying an AC voltage, uniform light emission can be observed only in the case where the anode has a plus polarity and where the cathode has a minus polarity. A waveform of an alternating current applied may be optional.

### Application of the Organic EL Device

The organic EL device of the present invention can be applied to products to which a high luminance and a high current efficiency are required even at a low voltage. The application examples thereof include, display units, displays, lighting instruments, printer light sources, backlights for liquid crystal display equipments and the like, and the organic EL device of the present invention can be applied as well to the fields such as indicators, advertising displays, interiors and the like. The display units include flat panel displays which save energy and have a high visibility. In the case of the printer light sources, the organic EL device of the present invention can be used as a light source for laser beam printers. Further, using the device of the present invention makes it possible to reduce a volume of the equipments to a large extent. In the case of the lighting instruments and the backlights, the energy saving effect can be expected by using the organic EL device of the present invention.

### EXAMPLES

The present invention shall be explained below in details with reference to examples, but the present invention shall not be restricted to the examples shown below as long as they do not exceed the scope of the present invention.

### Synthetic Example 1 Synthesis of 3,4,5,6-tetrafluoro-N¹,N⁸-bis(4-isopropylphenyl)-N¹, N⁸-di-p-tolyldibenzo[c,g]-triphenylene-1,8-diamine (1-84)

### 1-1. Synthesis of 3,4,5,6-tetrafluorodibenzo[c,g]triphenylene

A solution of 3.00 g (7.28 mmol) of 2,2′-dibromo-[1,1′]binaphthyl and 70 mL of anhydrous tetrahydrofuran was cooled down to −70° C., and a 2.67 M n-butyllithium hexane solution was dropwise added thereto in 5 minutes and stirred at −70° C. for 80 minutes. Hexafluorobenzene 0.98 mL (8.58 mmol) was dropwise added to the above mixture, and the solution was gradually heated up to room temperature and stirred for 5 hours. The reaction mixture was poured into water and extracted three times with dichloromethane. After separating the solution, the organic layer was dried on magnesium sulfate, and the solvent was removed by distillation under reduced pressure. A crude product obtained was refined by column chromatograph (silica gel: hexane→hexane/ethyl acetate=10/1) to obtain 1.96 g (yield: 67%) of the product.

### 1-2. Synthesis of 1,8-dibromo-3,4,5,6-tetrafluorodibenzo-[c,g]triphenylene

A piece (very small amount) of iodine was added to a mixed solution of 4.65 g (15.80 mmol) of 3,4,5,6-tetrafluorodibenzo[c,g]triphenylene and chloroform, and 1.70 ml (33.17 mmol) of bromine was dropwise added thereto and stirred at room temperature for 9 hours. After left standing overnight, a sodium hydrogensulfite aqueous solution was added to the reaction mixture, and the solution was extracted with dichloromethane. After separating the solution, the organic phase was concentrated and refined by flash chromatograph (silica gel: hexane/dichloromethane=10/1) to obtain 6.71 g (yield: 93.9%) of 1,8-dibromo-3,4,5,6-tetrafluorodibenzo-[c,g]triphenylene.

### 1-3. Synthesis of 3,4,5,6-tetrafluoro-N¹,N⁸-bis(4-isopropylphenyl)-N¹,N⁸-di-p-tolyldibenzo[c,g]triphenylene-1,8-diamine (1-84)

Tri-t-butylphosphine 0.14 g (0.67 mmol) was added to a suspension of 3.20 g (7.08 mmol) of 1,8-dibromodibenzo[c,g]-triphenylene, 5.71 g (21.23 mmol) of N-(4-isopropylphenyl)-4-tolylamine, 0.10 g (0.42 mmol) of palladium acetate, 2.04 g (21.23 mmol) of sodium t-butoxide and 50 ml of toluene under argon atmosphere to react them for 10 hours under refluxing by heating. Methanol was added to the reaction mixture, and a solid matter deposited was obtained by filtering and washed with methanol. The solid matter thus obtained was recrystallized from dichloromethane and methanol and washed with methanol and dichloromethane to obtain 0.69 g (yield: 37.9%) of 3,4,5,6-tetrafluoro-N¹,N⁸-bis(4-isopropylphenyl)-N¹,N⁸-di-p-tolyldibenzo[c,g]triphenylene-1,8-diamine (1-84).

A UV absorption spectrum in a toluene solution, a fluorescence emission spectrum and ¹H-NMR of the compound thus obtained were measured. The results thereof are shown below. UV (PhMe); λₘₐₓ, 438 (ε 4.28), FL(PhMe, λₑₓ=296 nm); λₘₐₓ, 509 nm

### Example 1

### Production of Organic EL Device

A transparent electrode having a film thickness of 120 nm comprising indium tin oxide was provided on a glass substrate having a size of 25 mm×75 mm×1.1 mm thickness. The above glass substrate was washed by irradiating with a UV ray and ozone and then loaded in a vacuum vapor deposition apparatus.

First, N′,N″-bis[4-(diphenylamino)phenyl]-N′,N″-diphenylbiphenyl-4,4′-diamine was deposited as a hole injecting layer in a thickness of 60 nm, and then N,N,N′,N′-tetrakis(4-biphenyl)-4,4′-benzidine was deposited thereon as a hole transporting layer in a thickness of 20 nm. Next, 10-(2-naphthalene-1-yl)phenyl-9-(naphthalene-2-yl)anthracene and the compound (1-84) described above were deposited at the same time in a weight ratio of 40:2 to form a light emitting layer having a thickness of 40 nm.

Next, tris(8-hydroxyquinolinato)aluminum was deposited thereon as an electron injecting layer in a thickness of 20 nm. Next, lithium fluoride was deposited thereon in a thickness of 1 nm, and then aluminum was deposited thereon in a thickness of 150 nm. This aluminum/lithium fluoride functions as a cathode. Thus, an organic EL device was prepared.

Next, the above device was subjected to an electric current test to find that emission of pure blue color light (emission maximum wavelength: 525 nm) having a current efficiency of 3.0 cd/A and a light emitting luminance of 300 cd/m² was obtained at a voltage of 6.5 V and a current density of 10 mA/cm². A direct current test thereof was carried out at an initial light emitting luminance of 100 cd/m² to find that the half lifetime was 10,000 hours or longer.

### Example 2

### Production of Organic EL Device

An organic EL device was prepared in the same manner as in Example 1, except that the compound (1-55) was used in place of the compound (1-84).

The above device was subjected to an electric current test to find that emission of pure blue color light (emission maximum wavelength: 430 nm) having a current efficiency of 2.0 cd/A and a light emitting luminance of 200 cd/m² was obtained at a voltage of 6.5 V and a current density of 10 mA/cm². A direct current test thereof was carried out at an initial light emitting luminance of 50 cd/m² to find that the half lifetime was 10,000 hours or longer.

### Comparative Example 1

An organic EL device was prepared in the same manner as in Example 1, except that dibenzo[c,g]triphenylene was used in place of the compound (1-84).

The above device was subjected to an electric current test to find that emission of pure blue color light having a current efficiency of 0.9 cd/A was obtained at a voltage of 6.3 V and a current density of 10 mA/cm². A direct current test thereof was carried out at an initial light emitting luminance of 50 cd/m² to find that the half lifetime was as short as 200 hours.

### INDUSTRIAL APPLICABILITY

As explained above in details, the organic EL device of the present invention in which a light emitting layer contains at least one selected from the dibenzo[c,g]triphenylene derivatives represented by Formula (1) described above and at least one selected from the compounds having the structures represented by Formulas (2a) to (2d) described above is excellent in a color purity and has a high light emitting luminance and a long lifetime. Accordingly, the organic EL device of the present invention is highly practical and useful as a light source for a flat light emitting body of wall-hung TV, a backlight of displays and the like. It can also be used as an organic EL device, an hole injecting and transporting material and an electron transporting material for electrophotographic sensitizers and organic semiconductors

1. A dibenzo[c,g]triphenylene derivative represented by the following Formula (1): 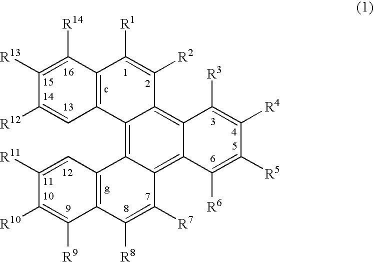
wherein R¹ to R¹⁴ each represent independently a hydrogen atom, a substituted or non-substituted alkyl group having 1 to 50 carbon atoms, a substituted or non-substituted alkoxyalkyl group having 1 to 50 carbon atoms, a substituted or non-substituted aralkyl group having 7 to 50 carbon atoms, a substituted or non-substituted cycloalkyl group having 5 to 50 carbon atoms, a substituted or non-substituted alkoxy group having 1 to 50 carbon atoms, a substituted or non-substituted alkylamino group having 1 to 20 carbon atoms, a substituted or non-substituted alkoxycarbonyl group having 1 to 50 carbon atoms, a substituted or non-substituted aryl group having 5 to 50 carbon atoms, a substituted or non-substituted aryloxy group having 5 to 50 carbon atoms, a substituted or non-substituted arylthio group having 5 to 50 ring carbon atoms, a substituted or non-substituted arylamino group having 5 to 50 carbon atoms, a substituted or non-substituted heteroaryl group having 5 to 50 carbon atoms, a substituted or non-substituted heteroaryloxy group having 5 to 50 carbon atoms, a substituted or non-substituted heteroarylthio group having 5 to 50 carbon atoms, a substituted or non-substituted heteroarylamino group having 5 to 50 carbon atoms, a substituted or non-substituted silyl group having 1 to 20 carbon atoms, a halogen atom, a cyano group or a nitro group; among the groups represented by R¹ to R¹⁴, the adjacent groups may be combined with each other when they are not hydrogens to form saturated or unsaturated cyclic structures, and these cyclic structures may be substituted.

2. The dibenzo[c,g]triphenylene derivative as described in claim 1, wherein R¹ and R⁸ in Formula (1) each are independently a substituted or non-substituted aryl group having 5 to 50 carbon atoms.

3. The dibenzo[c,g]triphenylene derivative as described in claim 1, wherein R¹ and R⁸ in Formula (1) each are independently a substituted or non-substituted arylamino group having 5 to 50 carbon atoms.

4. A material for an organic electroluminescence device comprising the dibenzo[c,g]triphenylene derivative as described in claim 1.

5. A light emitting material for an organic electroluminescence device comprising the dibenzo[c,g]triphenylene derivative as described in claim 1.

6. An organic electroluminescence device in which an organic compound layer comprising a single layer or plural layers including at least a light emitting layer is interposed between a pair of electrodes, wherein at least one layer in the organic compound layer comprises at least one kind of the dibenzo[c,g]triphenylene derivative as described in claim 1.

7. The organic electroluminescence device as described in claim 6, wherein the light emitting layer comprises the at least one kind of the dibenzo[c,g]triphenylene derivative.

8. The organic electroluminescence device as described in claim 7, wherein the light emitting layer further comprises at least one compound selected from compounds represented by the following Formulas (2a) to (2d): 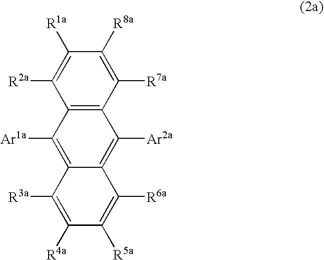
wherein Ar^{1a} and Ar^{2a} each are independently a group derived from a substituted or non-substituted aromatic ring having 6 to 20 ring carbon atoms; the aromatic ring described above may be substituted with at least one substituent; the substituent described above is selected from a substituted or non-substituted aryl group having 6 to 50 ring carbon atoms, a substituted or non-substituted alkyl group having 1 to 50 carbon atoms, a substituted or non-substituted cycloalkyl group having 3 to 50 carbon atoms, a substituted or non-substituted alkoxy group having 1 to 50 carbon atoms, a substituted or non-substituted aralkyl group having 6 to 50 carbon atoms, a substituted or non-substituted aryloxy group having 5 to 50 ring carbon atoms, a substituted or non-substituted arylthio group having 5 to 50 ring carbon atoms, a substituted or non-substituted alkoxycarbonyl group having 1 to 50 carbon atoms, a substituted or non-substituted silyl group, a carboxy group, a halogen atom, a cyano group, a nitro group and a hydroxy group; when the aromatic ring described above is substituted with two or more substituents, the substituents described above may be the same or different, and the substituents which are adjacent may be combined with each other to form a saturated or unsaturated cyclic structure; R^{1a} to R^{8a} each are selected independently from a hydrogen atom, a substituted or non-substituted aryl group having 6 to 50 ring carbon atoms, a substituted or non-substituted heteroaryl group having 5 to 50 ring carbon atoms, a substituted or non-substituted alkyl group having 1 to 50 carbon atoms, a substituted or non-substituted cycloalkyl group having 3 to 50 carbon atoms, a substituted or non-substituted alkoxy group having 1 to 50 carbon atoms, a substituted or non-substituted aralkyl group having 6 to 50 carbon atoms, a substituted or non-substituted aryloxy group having 5 to 50 ring carbon atoms, a substituted or non-substituted arylthio group having 5 to 50 ring carbon atoms, a substituted or non-substituted alkoxycarbonyl group having 1 to 50 carbon atoms, a substituted or non-substituted silyl group, a carboxy group, a halogen atom, a cyano group, a nitro group and a hydroxy group; 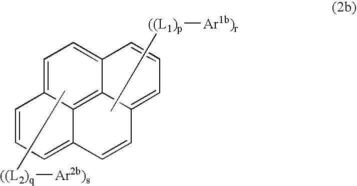
wherein Ar^{1b} and Ar^{2b} each are independently a substituted or non-substituted aryl group having 6 to 50 ring carbon atoms; L₁ and L₂ each are selected independently from a substituted or non-substituted phenylene group, a substituted or non-substituted naphthalenylene group, a substituted or non-substituted fluorenylene group and a substituted or non-substituted dibenzosilolylene group; p and q are an integer of 0 to 2, and r and s are an integer of 1 to 4; L₁ or Ar^{1b} is boded to any of 1- to 5-positions of pyrene, and L₂ or Ar^{2b} is boded to any of 6- to 10-positions of pyrene; 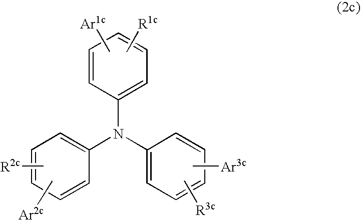
wherein Ar^{1c}, Ar^{2c} and Ar^{3c} each are selected independently from a group having an anthracene structure, a group having a phenanthrene structure, a group having a pyrene structure, a group having a fluorene structure, and a group having a perylene structure; and R^{1c}, R^{2c} and R^{3c} each represent independently a hydrogen atom or a substituent; 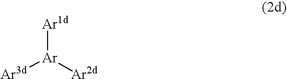
wherein Ar^{1d}, Ar^{2d} and Ar^{3d} each represent independently an aryl group having 6 to 50 ring carbon atoms; the aryl group described above may be substituted with at least one substituent; at least one of Ar^{1d}, Ar^{2d}, Ar^{3d} and the substituents present in these aryl groups has a fused ring aryl structure having 10 to 20 ring carbon atoms or a fused ring heteroaryl structure having 6 to 20 ring carbon atoms; and Ar represents a trivalent group derived from an aromatic ring or an aromatic heterocycle.

9. The organic electroluminescence device as described in claim 6, wherein the light emitting layer comprises 0.01 to 20% by weight of the dibenzo[c,g]triphenylene derivative.

10. The organic electroluminescence device as described in claim 6, wherein a layer selected from a chalcogenide layer, a halogenated metal layer and a metal oxide layer is provided on at least one surface of a pair of the electrodes.

11. An equipment comprising the organic electroluminescence device as described in any of claims 6 to 10.

12. A light emitting organic solution comprising the dibenzo[c,g]triphenylene derivative as described in claim 1.

13. A light emitting organic solution comprising:
at least one kind of a dibenzo[c,g]triphenylene derivative represented by the following Formula (1): 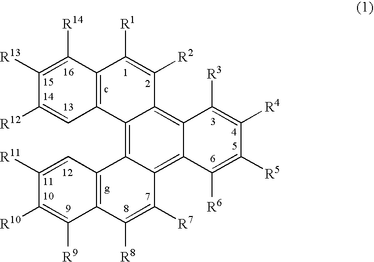
wherein R¹ to R¹⁴ each represent independently a hydrogen atom, a substituted or non-substituted alkyl group having 1 to 50 carbon atoms a substituted or non-substituted alkoxyalkyl group having 1 to 50 carbon atoms, a substituted or non-substituted aralkyl group having 7 to 50 carbon atoms, a substituted or non-substituted cycloalkyl group having 5 to 50 carbon atoms, a substituted or non-substituted alkoxy group having 1 to 50 carbon atoms, a substituted or non-substituted alkylamino group having 1 to 20 carbon atoms, a substituted or non-substituted alkoxycarbonyl group having 1 to 50 carbon atoms, a substituted or non-substituted aryl group having 5 to 50 carbon atoms, a substituted or non-substituted aryloxy group having 5 to 50 carbon atoms, a substituted or non-substituted arylthio group having 5 to 50 ring carbon atoms, a substituted or non-substituted arylamino group having 5 to 50 carbon atoms, a substituted or non-substituted heteroaryl group having 5 to 50 carbon atoms, a substituted or non-substituted heteroaryloxy group having 5 to 50 carbon atoms, a substituted or non-substituted heteroarylthio group having 5 to 50 carbon atoms, a substituted or non-substituted heteroarylamino group having 5 to 50 carbon atoms, a substituted or non-substituted silyl group having 1 to 20 carbon atoms, a halogen atom, a cyano group or a nitro group; among the groups represented by R¹ to R¹⁴, the adjacent groups may be combined with each other when they are not hydrogens to form saturated or unsaturated cyclic structures, and these cyclic structures may be substituted; and
at least one compound selected from the compounds represented by Formulas (2a) to (2d) as described in claim 8.
